SEQUENCE LISTING (125 sequences)

SEQ ID NO: 1 - protein, 21 aa, Artificial Sequence
  source: /note=“Description of Artificial Sequence: Synthetic peptide”
MALPVTALLLPLALLLHAARP

SEQ ID NO: 2 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 3 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 4 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 5 - protein, 10 aa, Artificial Sequence
  source: /note=“Description of Artificial Sequence: Synthetic peptide”
GGGGSGGGGS

SEQ ID NO: 6 - protein, 24 aa, Artificial Sequence
  source: /note=“Description of Artificial Sequence: Synthetic peptide”
IYIWAPLAGTCGVLLLSLVITLYC

SEQ ID NO: 7 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 8 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 9 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 13 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 15 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 16 - DNA, 30 bp, Artificial Sequence
  source: /note=“Description of Artificial Sequence: Synthetic oligonucleotide”
ggtggcggaggttctggaggtggaggttcc

SEQ ID NO: 17 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 18 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Artificial Sequence
  source: /note=“Description of Artificial Sequence: Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 24 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 5 aa, Artificial Sequence
  source: /note=“Description of Artificial Sequence: Synthetic peptide”
GGGGS

SEQ ID NO: 26 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ Repeating units”
GGGGSGGGGSGGGGSGGGGSGGGGSGGGGS

SEQ ID NO: 27 - protein, 20 aa, Artificial Sequence
  source: /note=“Description of Artificial Sequence: Synthetic peptide”
GGGGSGGGGSGGGGSGGGGS

SEQ ID NO: 28 - protein, 15 aa, Artificial Sequence
  source: /note=“Description of Artificial Sequence: Synthetic peptide”
GGGGSGGGGSGGGGS

SEQ ID NO: 29 - protein, 4 aa, Artificial Sequence
  source: /note=“Description of Artificial Sequence: Synthetic peptide”
GGGS

SEQ ID NO: 30 - RNA, 5000 nt, Artificial Sequence
  source: /note=“Description of Artificial Sequence: Synthetic polynucleotide”
  misc_feature (1)..(5000): /note=“This sequence may encompass 50-5000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 32 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, Artificial Sequence; identical to SEQ ID NO: 30
  source: /note=“Description of Artificial Sequence: Synthetic polynucleotide”
  misc_feature (1)..(5000): /note=“This sequence may encompass 100-5000 nucleotides”

SEQ ID NO: 34 - RNA, 400 nt, Artificial Sequence
  source: /note=“Description of Artificial Sequence: Synthetic polynucleotide”
  misc_feature (1)..(400): /note=“This sequence may encompass 100-400 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 41 aa, Artificial Sequence
  source: /note=“Description of Artificial Sequence: Synthetic polypeptide”
RSKRSRLLHSDYMNMTPRRPGPTRKHYQPYAPPRDFAAYRS

SEQ ID NO: 37 - DNA, 123 bp, Artificial Sequence; identical to SEQ ID NO: 19
  source: /note=“Description of Artificial Sequence: Synthetic polynucleotide”

SEQ ID NO: 38 - protein, 35 aa, Artificial Sequence
  source: /note=“Description of Artificial Sequence: Synthetic polypeptide”
TKKKYSSSVHDPNGEYMFMRAVNTAKKSRLTDVTL

SEQ ID NO: 39 - DNA, 105 bp, Artificial Sequence
  source: /note=“Description of Artificial Sequence: Synthetic polynucleotide”
acaaaaaagaagtattcatccagtgtgcacgaccctaacggtgaatacatgttcatgagagcagtgaacacagccaaaaaatccagactcacagatgtgacccta

SEQ ID NO: 40 - DNA, 30 bp, Artificial Sequence
  source: /note=“Description of Artificial Sequence: Synthetic oligonucleotide”
ggtggcggaggttctggaggtgggggttcc

SEQ ID NO: 41 - protein, 4 aa, Artificial Sequence
  source: /note=“Description of Artificial Sequence: Synthetic peptide”
  source: /note=“See specification as filed for detailed description of substitutions and preferred embodiments”
GGGS

SEQ ID NO: 42 - protein, 40 aa, Artificial Sequence
  source: /note=“Description of Artificial Sequence: Synthetic polypeptide”
  SITE (1)..(40): /note=“This sequence may encompass 1-10 ′Gly Gly Gly Ser′ Repeating units”
GGGSGGGSGGGSGGGSGGGSGGGSGGGSGGGSGGGSGGGS

SEQ ID NO: 43 - protein, 18 aa, Artificial Sequence
  source: /note=“Description of Artificial Sequence: Synthetic peptide”
GSTSGSGKPGSGEGSTKG

SEQ ID NO: 44-107 - skipped (64 entries)

SEQ ID NO: 108 - protein, 1132 aa, Homo sapiens [sequence omitted]

SEQ ID NO: 109 - DNA, 521 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 110 - DNA, 118 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtg

SEQ ID NO: 111 - DNA, 221 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 112 - DNA, 324 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 113 - DNA, 422 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 114 - protein, 132 aa, Artificial Sequence
  source: /note=“Description of Artificial Sequence: Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 115 - protein, 108 aa, Artificial Sequence
  source: /note=“Description of Artificial Sequence: Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 116 - protein, 93 aa, Artificial Sequence [sequence omitted]

SEQ ID NO: 117 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 118 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 119 - protein, 95 aa, Artificial Sequence
  source: /note=“Description of Artificial Sequence: Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 120 - protein, 95 aa, Artificial Sequence
  source: /note=“Description of Artificial Sequence: Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 121 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 122 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 123 - protein, 465 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 125 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2021/0171909 A1
(43) Pub. Date: Jun. 10, 2021

(54) METHODS OF MAKING CHIMERIC ANTIGEN RECEPTOR?EXPRESSING CELLS

(71) Applicant: Novartis AG, Basel (CH)

(72) Inventor: Tatiana Golovina, Havertown, PA (US)

(21) Appl. No.: 17/271,507

(22) PCT Filed: Aug. 30, 2019
(86) PCT No.: PCT/US2019/049123

§ 371 Date: Feb. 25, 2021

Related U.S. Application Data
(60) Provisional application No. 62/726,164, filed on Aug. 31, 2018.

(51) Int. Cl. C12N 5/0783; C07K 14/725; G01N 33/50; C07K 14/705; A61K 35/17; C07K 16/28; A61P 35/02

(52) U.S. Cl. CPC C12N 5/0636; C07K 2319/33; G01N 33/505; C07K 14/70578; A61K 35/17; C07K 16/2803; A61P 35/02; C12N 2501/2302; C12N 2501/2315; C12N 2501/2321; C12N 2501/51; C12N 2501/505; C07K 2319/03; C07K 2319/02; C07K 14/7051

(57) ABSTRACT

The invention provides methods of making immune effector cells (e.g., T cells, NK cells) that express a chimeric antigen receptor (CAR), and compositions generated by such methods.

### RELATED APPLICATION

This application claims priority to U.S. Ser. No. 62/726,164 filed Aug. 31, 2018, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 13, 2019, is named N2067-7159WO_SL.txt and is 90,588 bytes in size.

### FIELD OF THE INVENTION

The present invention relates generally to methods of making immune effector cells (e.g., T cells or NK cells) engineered to express a Chimeric Antigen Receptor (CAR), and compositions comprising the same.

### BACKGROUND OF THE INVENTION

Adoptive cell transfer (ACT) therapy with T cells, especially with T cells transduced with Chimeric Antigen Receptors (CARs), has shown promise in several hematologic cancer trials. The manufacture of gene-modified T cells is currently a complex process. There exists a need for methods and processes to improve production of the CAR-expressing cell therapy product, enhance product quality, and maximize the therapeutic efficacy of the product.

### SUMMARY OF THE INVENTION

The present disclosure pertains to methods of making immune effector cells (e.g., T cells or NK cells) engineered to express a CAR, and compositions generated using such methods. Also disclosed are methods of using such compositions for treating a disease, e.g., cancer, in a subject.

In one aspect, this invention features a method of making a population of cells (e.g., T cells) that express a chimeric antigen receptor (CAR), the method comprising: (i) contacting a population of cells (e.g., T cells) with one, two, or all three of IL-2, IL-15, and IL-21, and (ii) contacting the population of cells (e.g., T cells) with a nucleic acid molecule (e.g., a DNA or RNA molecule) encoding the CAR, thereby providing a population of cells (e.g., T cells) comprising the nucleic acid molecule. In some embodiments, the method further comprises: (iii) contacting (e.g., binding) the population of cells (e.g., T cells) with an agent that stimulates a CD3/TCR complex and/or an agent that stimulates a costimulatory molecule, optionally wherein: the agent that stimulates a CD3/TCR complex is an agent that stimulates CD3 (e.g., the agent that stimulates a CD3/TCR complex comprises an anti-CD3 antibody), and the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof (e.g., the agent that stimulates a costimulatory molecule comprises an anti-CD28 antibody). In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix. In some embodiments, step (iii) comprises contacting (e.g., binding) the population of cells (e.g., T cells) with T Cell TransAct™. In some embodiments, step (ii) is performed after step (i), e.g., about 0.5, 1, 1.5, or 2 days after the beginning of step (i), e.g., about 1 day after the beginning of step (i). In some embodiments, step (ii) is performed no more than 1, 2, 3, 4, 5, or 6 hours after the beginning of step (i). In some embodiments, step (i) and step (ii) are performed simultaneously. In some embodiments, step (iii) is performed together with step (i), or no more than 1, 2, 3, 4, 5, or 6 hours prior to or after step (i).

In one aspect, this invention features a method of making a population of cells (e.g., T cells) that express a chimeric antigen receptor (CAR), the method comprising: (i) contacting a population of cells (e.g., T cells) with IL-2 and one or both of: IL-15 and IL-21, and (ii) contacting the population of cells (e.g., T cells) with a nucleic acid molecule (e.g., a DNA or RNA molecule) encoding the CAR, thereby providing a population of cells (e.g., T cells) comprising the nucleic acid molecule, wherein the population of cells at the beginning of step (i) has one or more (e.g., 2, 3, 4, 5, 6, 7, 8, 9, 10, 11, 12, 13, 14, or all) of the following properties:

(1) the population of cells at the beginning of step (i) does not expand or expands for no more than 5, 6, 7, 8, or 9-fold over 8-11 days using the Bead CART cell manufacturing process described in Example 1,

(2) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than 5, 6, 7, 8, 9, 10, 15, 20, 25, 30, 40, 50, or 60%, e.g., lower than 10%,

(3) the percentage of naïve T cells and/or Tscm among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, or 90% lower than the corresponding value in a reference population of cells,

(4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than 20, 25, 30, 40, 50, 60, 70, 80, 90, or 95%, e.g., higher than 50%,

(5) the percentage of Teff cells and/or Tem cells among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 600, 700, 800, or 900% higher than the corresponding value in a reference population of cells,

(6) the percentage of CD28+CD4+ T cells among CD4+ T cells in the population of cells at the beginning of step (i) is no more than 40, 45, 50, 55, 60, 65, 70, 75, 80, 85, 90, or 95%, e.g., no more than 50%,

(7) the percentage of CD28+CD8+ T cells among CD8+ T cells in the population of cells at the beginning of step (i) is no more than 30, 35, 40, 45, 50, 55, 60, 65, or 70%, e.g., no more than 50%,

(8) the percentage of CD28+ T cells among T cells, the percentage of CD28+CD4+ T cells among CD4+ T cells, or the percentage of CD28+CD8+ T cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CART cell manufacturing process described in Example 1), e.g., at least 20, 30, 40, 50, 60, 70, 80, or 90% lower than the corresponding value in a reference population of cells,

(9) the ratio of CD4+ T cells to CD8+ T cells in the population of cells at the beginning of step (i) is lower than 0.5, 0.8, 1, 1.2, or 1.5,

(10) the ratio of CD4+ T cells to CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, or 99% lower than the corresponding value in a reference population of cells,

(11) the percentage of CD4+ senescent cells (e.g., CD28−CD27−CD57+CD4+ senescent cells) among CD4+ T cells in the population of cells at the beginning of step (i) is more than 2, 5, 10, 15, 20, 30, 40, 50, 60, 70, 80, 90 or 95%,

(12) the percentage of CD8+ senescent cells (e.g., CD28−CD27−CD57+CD8+ senescent cells) among CD8+ T cells in the population of cells at the beginning of step (i) is more than 20, 25, 30, 35, 40, 50, 60, 70, 80, 90 or 95%,

(13) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000, or 9500% higher than the corresponding value in a reference population of cells,

(14) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than 10, 20, 30, 40, 50, 60, 70, 80 or 90%, and

(15) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000 or 9000% higher than the corresponding value in a reference population of cells.

In some embodiments, the population of cells at the beginning of step (i) does not expand or expands for no more than 5, 6, 7, 8, or 9-fold over 8-11 days using the Bead CAR T cell manufacturing process described in Example 1. In some embodiments, the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than 5, 6, 7, 8, 9, 10, 15, 20, 25, 30, 40, 50, or 60%, e.g., lower than 10%. In some embodiments, the percentage of naïve T cells and/or Tscm among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, or 90% lower than the corresponding value in a reference population of cells. In some embodiments, the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than 20, 25, 30, 40, 50, 60, 70, 80, 90, or 95%, e.g., higher than 50%. In some embodiments, the percentage of Teff cells and/or Tem cells among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 600, 700, 800, or 900% higher than the corresponding value in a reference population of cells. In some embodiments, the percentage of CD28+CD4+ T cells among CD4+ T cells in the population of cells at the beginning of step (i) is no more than 40, 45, 50, 55, 60, 65, 70, 75, 80, 85, 90, or 95%, e.g., no more than 50%. In some embodiments, the percentage of CD28+CD8+ T cells among CD8+ T cells in the population of cells at the beginning of step (i) is no more than 30, 35, 40, 45, 50, 55, 60, 65, or 70%, e.g., no more than 50%. In some embodiments, the percentage of CD28+ T cells among T cells, the percentage of CD28+CD4+ T cells among CD4+ T cells, or the percentage of CD28+CD8+ T cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, or 90% lower than the corresponding value in a reference population of cells. In some embodiments, the ratio of CD4+ T cells to CD8+ T cells in the population of cells at the beginning of step (i) is lower than 0.5, 0.8, 1, 1.2, or 1.5. In some embodiments, the ratio of CD4+ T cells to CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, or 99% lower than the corresponding value in a reference population of cells. In some embodiments, the percentage of CD4+ senescent cells (e.g., CD28−CD27−CD57+CD4+ senescent cells) among CD4+ T cells in the population of cells at the beginning of step (i) is more than 2, 5, 10, 15, 20, 30, 40, 50, 60, 70, 80, 90 or 95%. In some embodiments, the percentage of CD8+ senescent cells (e.g., CD28−CD27−CD57+CD8+ senescent cells) among CD8+ T cells in the population of cells at the beginning of step (i) is more than 20, 25, 30, 35, 40, 50, 60, 70, 80, 90 or 95%. In some embodiments, the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000, or 9500% higher than the corresponding value in a reference population of cells. In some embodiments, the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than 10, 20, 30, 40, 50, 60, 70, 80 or 90%. In some embodiments, the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000 or 9000% higher than the corresponding value in a reference population of cells.

In some embodiments, step (i) comprises contacting the population of cells (e.g., T cells) with IL-2, IL-15, and IL-21. In some embodiments, step (i) comprises contacting the population of cells (e.g., T cells) with IL-2 at about 10, 20, 30, 50, 60, 70, 80, 90, 100, 200, 300, 400, 500, 600, 700, or 800 U/mL. In some embodiments, step (i) comprises contacting the population of cells (e.g., T cells) with IL-15 at about 1, 2, 5, 10, 15, 20, 25, 30, 35, or 40 ng/mL. In some embodiments, step (i) comprises contacting the population of cells (e.g., T cells) with IL-21 at about 1, 2, 5, 10, 15, 20, 25, 30, 35, or 40 ng/mL.

In some embodiments, step (ii) is performed after step (i), e.g., about 0.5, 1, 1.5, or 2 days after the beginning of step (i), e.g., about 1 day after the beginning of step (i). In some embodiments, step (ii) is performed no more than 1, 2, 3, 4, 5, or 6 hours after the beginning of step (i). In some embodiments, step (i) and step (ii) are performed simultaneously.

In some embodiments, the nucleic acid molecule in step (ii) is on a viral vector, e.g., a lentiviral vector or a retroviral vector. In some embodiments, the nucleic acid molecule in step (ii) is an RNA molecule on a viral vector. In some embodiments, step (ii) comprises transducing the population of cells (e.g., T cells) with a viral vector comprising a nucleic acid molecule encoding the CAR.

In some embodiments, the method described herein further comprises (iii) contacting (e.g., binding) the population of cells (e.g., T cells) with an agent that stimulates a CD3/TCR complex and/or an agent that stimulates a costimulatory molecule. In some embodiments, the agent that stimulates a CD3/TCR complex is an agent that stimulates CD3 (e.g., the agent that stimulates a CD3/TCR complex comprises an anti-CD3 antibody). In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof (e.g., the agent that stimulates a costimulatory molecule comprises an anti-CD28 antibody). In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, step (iii) comprises contacting (e.g., binding) the population of cells (e.g., T cells) with T Cell TransAct™.

In one embodiment, the agent that stimulates a CD3/TCR complex does not comprise hydrogel. In one embodiment, the agent that stimulates a costimulatory molecule does not comprise hydrogel. In one embodiment, the agent that stimulates a CD3/TCR complex does not comprise alginate. In one embodiment, the agent that stimulates a costimulatory molecule does not comprise alginate.

In one embodiment, the agent that stimulates a CD3/TCR complex comprises hydrogel. In one embodiment, the agent that stimulates a costimulatory molecule comprises hydrogel. In one embodiment, the agent that stimulates a CD3/TCR complex comprises alginate. In one embodiment, the agent that stimulates a costimulatory molecule comprises alginate. In one embodiment, the agent that stimulates a CD3/TCR complex or the agent that stimulates a costimulatory molecule comprises MagCloudz™ from Quad Technologies.

In some embodiments, step (iii) is performed together with step (i), or no more than 1, 2, 3, 4, 5, or 6 hours prior to or after step (i). In some embodiments, step (iii) is performed together with step (ii), or no more than 1, 2, 3, 4, 5, or 6 hours prior to or after step (ii).

In some embodiments, the population of cells at the beginning of step (i) is isolated from apheresis material by negative selection. In some embodiments, the population of cells at the beginning of step (i) is isolated from apheresis material by reducing monocytes (e.g., CD14+ cells), B cells (e.g., CD19+ cells), and/or NK cells (e.g., CD56+ cells) from the apheresis material. In some embodiments, the population of cells at the beginning of step (i) is isolated from apheresis material using CliniMACS. In some embodiments, the apheresis material is leukapheresis material. In some embodiments, the apheresis material is fresh or frozen leukapheresis material.

In some embodiments, the apheresis, e.g., leukapheresis, material is isolated from a subject having cancer. In some embodiments, the cancer is a solid cancer, e.g., chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof. In some embodiments, the cancer is a liquid cancer, e.g., chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma. In some embodiments, the cancer is chronic lymphoblastic leukemia (CLL) or diffuse large B-cell lymphoma (DLBCL).

In some embodiments, the apheresis, e.g., leukapheresis, material is isolated from a subject, cryopreserved after being isolated from the subject, and thawed before the population of cells at the beginning of step (i) is isolated from the apheresis, e.g., leukapheresis material.

In some embodiments, the percentage of T cells in the apheresis, e.g., leukapheresis, material is no more than 1, 5, 10, 15, 20, 25, 30, 35, or 40%. In some embodiments, the percentage of T cells in the apheresis, e.g., leukapheresis, material is lower than the corresponding value in reference apheresis, e.g., leukapheresis, material (e.g., apheresis, e.g., leukapheresis, material from a healthy donor), e.g., at least 20, 30, 40, 50, 60, 70, 80, or 90% lower than the corresponding value in reference apheresis, e.g., leukapheresis, material.

In some embodiments, the method described herein further comprises after step (ii): (iv) expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells.

In some embodiments, step (i) comprises contacting the population of cells (e.g., T cells) with IL-2, IL-15, and IL-21. In some embodiments, the expansion of the population of expanded cells at the end of step (iv) relative to the population of cells at the beginning of step (i) is greater (e.g., at least 5, 10, 15, 20, 25, 30, 40, 50, 100, 200, 300, 400 or 500% greater) than the expansion of a population of cells made by an otherwise similar method in which the population of cells is not contacted with IL-2, IL-15, or IL-21, or is contacted with IL-2 but not IL-15 or IL-21, e.g., as measured by population doubling level (PDL), e.g., as assessed using methods described in Example 1 with respect to FIGS. 3-6, and 10. In some embodiments, the percentage of CD28+ T cells among T cells (e.g., the percentage of CD28+CD8+ T cells among CD8+ T cells, or the percentage of CD28+CD4+ T cells among CD4+ T cells) in the population of expanded cells at the end of step (iv) is higher (e.g., at least 5, 10, 15, 20, 25, 30, 40, 50, 60, 70, or 80% higher) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIG. 7. In some embodiments, the percentage of PD-1+CD8+ T cells among CD8+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9. In some embodiments, the percentage of LAG3+CD8+ T cells among CD8+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9. In some embodiments, the percentage of PD-1+LAG3+CD8+ T cells among CD8+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9. In some embodiments, the percentage of PD-1+CD4+ T cells among CD4+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9. In some embodiments, the percentage of LAG3+CD4+ T cells among CD4+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9. In some embodiments, the percentage of PD-1+LAG3+CD4+ T cells among CD4+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

In some embodiments, step (iii) comprises contacting the population of cells (e.g., T cells) with T Cell TransAct™. In some embodiments, the expansion of the population of expanded cells at the end of step (iv) relative to the population of cells at the beginning of step (i) is greater (e.g., at least 20, 30, 40, 50, 60, 70, 80, 90, 100, 200, 300, 400, or 500% greater) than the expansion of a population of cells made by an otherwise similar method in which the population of cells is contacted with an agent that stimulates a CD3/TCR complex and comprises a bead, and/or an agent that stimulates a costimulatory molecule and comprises a bead, e.g., e.g., as assessed using methods described in Example 1 with respect to FIGS. 4-6, and 10. In some embodiments, the percentage of CD28+ T cells among T cells (e.g., the percentage of CD28+CD8+ T cells among CD8+ T cells, or the percentage of CD28+CD4+ T cells among CD4+ T cells) in the population of expanded cells at the end of step (iv) is higher (e.g., at least 30, 35, 40, 50, 60, 70, 80, 90, 100, 200, 300, 400, or 500% higher) than the corresponding value in a population of cells made by an otherwise similar method in which the population of cells is contacted with an agent that stimulates a CD3/TCR complex and comprises a bead, and/or an agent that stimulates a costimulatory molecule and comprises a bead, e.g., as assessed using methods described in Example 1 with respect to FIG. 7. In some embodiments, the percentage of exhausted T cells among T cells (e.g., the percentage of PD-1+CD8+ T cells among CD8+ T cells, the percentage of LAG3+CD8+ T cells among CD8+ T cells, the percentage of PD-1+LAG3+CD8+ T cells among CD8+ T cells, the percentage of PD-1+CD4+ T cells among CD4+ T cells, the percentage of LAG3+CD4+ T cells among CD4+ T cells, or the percentage of PD-1+LAG3+CD4+ T cells among CD4+ T cells) in the population of expanded cells at the end of step (iv) is lower (e.g., at least 30, 35, 40, 45, 50, 55, 60, 65, 70, 75, 80, 85, 90, or 95% lower) than the corresponding value in a population of cells made by an otherwise similar method in which the population of cells is contacted with an agent that stimulates a CD3/TCR complex and comprises a bead, and/or an agent that stimulates a costimulatory molecule and comprises a bead, e.g., as assessed using methods described in Example 1 with respect to FIGS. 8-9.

In some embodiments, step (i) comprises contacting the population of cells (e.g., T cells) with IL-2, IL-15, and IL-21, and step (iii) comprises contacting (e.g., binding) the population of cells (e.g., T cells) with T Cell TransAct™. In some embodiments, the expansion of the population of expanded cells at the end of step (iv) relative to the population of cells at the beginning of step (i) is similar to or differs by no more than 5, 10, or 15% from the expansion of a reference population of cells (e.g., a population of cells from a healthy donor) made by the same method, e.g., as assessed using methods described in Example 1 with respect to FIG. 11.

In some embodiments, the method described herein further comprises step (v): contacting the population of cells (e.g., T cells) with IL-2 and one or both of: IL-15 and IL-21. In some embodiments, step (v) comprises contacting the population of cells (e.g., T cells) with IL-2, IL-15, and IL-21, at least 3 days after the beginning of step (i), e.g., at least 3, 4, 5, 6, 7, 8, 9, or 10 days after the beginning of step (i).

In one aspect, featured herein is a method of making a population of cells (e.g., T cells) that express a chimeric antigen receptor (CAR), the method comprising:

(a) providing apheresis, e.g., leukapheresis, material, optionally wherein the apheresis, e.g., leukapheresis, material is cryopreserved after being isolated from a subject, and thawed prior to step (b),

(b) isolating a population of cells (e.g., T cells) from the apheresis, e.g., leukapheresis, material using negative selection, e.g., by reducing monocytes (e.g., CD14+ cells), B cells (e.g., CD19+ cells), and/or NK cells (e.g., CD56+ cells), from the apheresis, e.g., leukapheresis, material, e.g., using CliniMACS,

(c) contacting the population of cells (e.g., T cells) with IL-2 and one or both of: IL-15 and IL-21, e.g., contacting the population of cells (e.g., T cells) with IL-2, IL-15, and IL-21,

(d) contacting (e.g., binding) the population of cells (e.g., T cells) with an agent that stimulates a CD3/TCR complex and/or an agent that stimulates a costimulatory molecule, wherein the agent that stimulates a CD3/TCR complex or the agent that stimulates a costimulatory molecule does not comprise a bead, e.g., contacting (e.g., binding) the population of cells (e.g., T cells) with T Cell TransAct™,

(e) contacting the population of cells (e.g., T cells) with a nucleic acid molecule (e.g., a DNA or RNA molecule) encoding the CAR, thereby providing a population of cells (e.g., T cells) comprising the nucleic acid molecule, and

(f) expanding the population of cells comprising the nucleic acid molecule for at least 3, 4, 5, 6, 7, 8, 9, or 10 days in vitro, e.g., expanding the population of cells comprising the nucleic acid molecule for about 4-10 days, to produce a population of expanded cells.

In one aspect, featured herein is a method of making a population of cells (e.g., T cells) that express a chimeric antigen receptor (CAR), the method comprising:

(a) providing apheresis, e.g., leukapheresis, material, optionally wherein the apheresis, e.g., leukapheresis, material is cryopreserved after being isolated from a subject, and thawed prior to step (b),

(b) isolating a population of cells (e.g., T cells) from the apheresis, e.g., leukapheresis, material using negative selection, e.g., by reducing monocytes (e.g., CD14+ cells), B cells (e.g., CD19+ cells), and/or NK cells (e.g., CD56+ cells), from the apheresis, e.g., leukapheresis, material, e.g., using CliniMACS,

(c) contacting the population of cells (e.g., T cells) with IL-2 and one or both of: IL-15 and IL-21, e.g., contacting the population of cells (e.g., T cells) with IL-2, IL-15, and IL-21,

(d) contacting (e.g., binding) the population of cells (e.g., T cells) with an agent that stimulates a CD3/TCR complex and/or an agent that stimulates a costimulatory molecule, wherein the agent that stimulates a CD3/TCR complex or the agent that stimulates a costimulatory molecule does not comprise a bead, e.g., contacting (e.g., binding) the population of cells (e.g., T cells) with T Cell TransAct™, and

(e) contacting the population of cells (e.g., T cells) with a nucleic acid molecule (e.g., a DNA or RNA molecule) encoding the CAR, thereby providing a population of cells (e.g., T cells) comprising the nucleic acid molecule.

Alternative methods resulting in the negative selection of T cells can also be used in the methods described herein. Additional bead-free reagents stimulating a CD3/TCR complex and/or a costimulatory molecule, including but not limited to CD28, can also be used in the methods described herein.

In some embodiments, the population of cells at the beginning of step (i) has one or more (e.g., 2, 3, 4, 5, 6, 7, 8, 9, 10, 11, 12, 13, 14, or all) of the following properties:

(1) the population of cells at the beginning of step (i) does not expand or expands for no more than 5, 6, 7, 8, or 9-fold over 8-11 days using the Bead CART cell manufacturing process described in Example 1,

(2) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than 5, 6, 7, 8, 9, 10, 15, 20, 25, 30, 40, 50, or 60%, e.g., lower than 10%,

(3) the percentage of naïve T cells and/or Tscm among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, or 90% lower than the corresponding value in a reference population of cells,

(4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than 20, 25, 30, 40, 50, 60, 70, 80, 90, or 95%, e.g., higher than 50%,

(5) the percentage of Teff cells and/or Tem cells among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 600, 700, 800 or 900% higher than the corresponding value in a reference population of cells,

(6) the percentage of CD28+CD4+ T cells among CD4+ T cells in the population of cells at the beginning of step (i) is no more than 40, 45, 50, 55, 60, 65, 70, 75, 80, 85, 90, or 95%, e.g., no more than 50%,

(7) the percentage of CD28+CD8+ T cells among CD8+ T cells in the population of cells at the beginning of step (i) is no more than 30, 35, 40, 45, 50, 55, 60, 65, or 70%, e.g., no more than 50%,

(8) the percentage of CD28+ T cells among T cells, the percentage of CD28+CD4+ T cells among CD4+ T cells, or the percentage of CD28+CD8+ T cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CART cell manufacturing process described in Example 1), e.g., at least 20, 30, 40, 50, 60, 70, 80, or 90% lower than the corresponding value in a reference population of cells,

(9) the ratio of CD4+ T cells to CD8+ T cells in the population of cells at the beginning of step (i) is lower than 0.5, 0.8, 1, 1.2, or 1.5,

(10) the ratio of CD4+ T cells to CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, or 99% lower than the corresponding value in a reference population of cells,

(11) the percentage of CD4+ senescent cells (e.g., CD28−CD27−CD57+CD4+ senescent cells) among CD4+ T cells in the population of cells at the beginning of step (i) is more than 2, 5, 10, 15, 20, 30, 40, 50, 60, 70, 80, 90 or 95%,

(12) the percentage of CD8+ senescent cells (e.g., CD28−CD27−CD57+CD8+ senescent cells) among CD8+ T cells in the population of cells at the beginning of step (i) is more than 20, 25, 30, 35, 40, 50, 60, 70, 80, 90 or 95%,

(13) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 60, 70, 80, 90 or 95% higher than the corresponding value in a reference population of cells,

(14) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than 10, 20, 30, 40, 50, 60, 70, 80 or 90%, and

(15) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000 or 9000% higher than the corresponding value in a reference population of cells.

In one aspect, disclosed herein is a method of evaluating or predicting suitability of a population of cells (e.g., T cells) for chimeric antigen receptor (CAR) manufacturing, the method comprising: acquiring a value for one or more (e.g., 2, 3, 4, 5, or all) of the following from the population of cells (e.g., T cells): (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, (3) the percentage of CD28+ T cells among T cells, the percentage of CD28+CD4+ T cells among CD4+ T cells, or the percentage of CD28+CD8+ T cells among CD8+ T cells, (4) the ratio of CD4+ T cells to CD8+ T cells, (5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and (6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, wherein: (a) a decrease in the value of one, two, or all of (1), (3), and (4) as compared to a reference value, e.g., a healthy donor reference value, is indicative or predicative of decreased suitability of the population of cells (e.g., T cells) for CAR manufacturing, (b) an increase in the value of one, two, or all of (1), (3), and (4) as compared to a reference value, e.g., a healthy donor reference value, is indicative or predicative of increased suitability of the population of cells (e.g., T cells) for CAR manufacturing, (c) an increase in the value of one, two, or all of (2), (5), and (6) as compared to a reference value, e.g., a healthy donor reference value, is indicative or predicative of decreased suitability of the population of cells (e.g., T cells) for CAR manufacturing, or (d) a decrease in the value of one, two, or all of (2), (5), and (6) as compared to a reference value, e.g., a healthy donor reference value, is indicative or predicative of increased suitability of the population of cells (e.g., T cells) for CAR manufacturing, thereby evaluating or predicting suitability of the population of cells (e.g., T cells) for CAR manufacturing.

In some embodiments, the method comprises acquiring the value of (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells. In some embodiments, the value being lower than 8, 9, 10, 11, 12, 15, or 20%, e.g., 10%, indicates or predicts decreased suitability of the population of cells (e.g., T cells) for CAR manufacturing. In some embodiments, the value being lower than 8, 9, 10, 11, 12, 15, or 20%, e.g., 10%, indicates or predicts the population of cells (e.g., T cells) shows more expansion using the method of any one of claims 1-27, e.g., the bead-free stimulation and cytokine (BFSC) process described in Example 1, compared with the Bead CAR T cell manufacturing process described in Example 1.

In some embodiments, the method comprises acquiring the value of (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells. In some embodiments, the value being higher than 40, 45, 50, 55, or 60%, e.g. 50%, indicates or predicts decreased suitability of the population of cells (e.g., T cells) for CAR manufacturing. In some embodiments, the value being higher than 40, 45, 50, 55, or 60%, e.g. 50%, indicates or predicts the population of cells (e.g., T cells) shows more expansion using the method of any one of claims 1-27, e.g., the bead-free stimulation and cytokine (BFSC) process described in Example 1, compared with the Bead CAR T cell manufacturing process described in Example 1.

In some embodiments, the method comprises acquiring the value of (3) the percentage of CD28+ T cells among T cells, the percentage of CD28+CD4+ T cells among CD4+ T cells, or the percentage of CD28+CD8+ T cells among CD8+ T cells. In some embodiments, the value being lower than 40, 45, 50, 55, or 60%, e.g. 50%, indicates or predicts decreased suitability of the population of cells (e.g., T cells) for CAR manufacturing. In some embodiments, the value being lower than 40, 45, 50, 55, or 60%, e.g. 50%, indicates or predicts the population of cells (e.g., T cells) shows more expansion using the method of any one of claims 1-27, e.g., the bead-free stimulation and cytokine (BFSC) process described in Example 1, compared with the Bead CAR T cell manufacturing process described in Example 1.

In some embodiments, the method comprises acquiring the value of (4) the ratio of CD4+ T cells to CD8+ T cells. In some embodiments, the value being lower than 0.5, 0.8, 1, 1.2, or 1.5 indicates or predicts decreased suitability of the population of cells (e.g., T cells) for CAR manufacturing.

In some embodiments, the method comprises acquiring the value of (5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells. In some embodiments, the value being higher than 20, 25, 30, 35, 40, 50, 60, 70, 80, 90 or 95% indicates or predicts decreased suitability of the population of cells (e.g., T cells) for CAR manufacturing.

In some embodiments, the method comprises acquiring the value of (6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells. In some embodiments, the value being higher than 10, 20, 30, 40, 50, 60, 70, 80 or 90% indicates or predicts decreased suitability of the population of cells (e.g., T cells) for CAR manufacturing.

In one aspect, disclosed herein is a method of making a population of cells (e.g., T cells) that express a chimeric antigen receptor (CAR), the method comprising: responsive to a decreased value for one, two, or all of the following in the population of cells (e.g., T cells): (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, (2) the percentage of CD28+ T cells among T cells, the percentage of CD28+CD4+ T cells among CD4+ T cells, or the percentage of CD28+CD8+ T cells among CD8+ T cells, and (3) the ratio of CD4+ T cells to CD8+ T cells, as compared to a reference value, e.g., a healthy donor reference value, performing: (a) contacting the population of cells (e.g., T cells) with IL-2 and one or both of: IL-15 and IL-21, e.g., contacting the population of cells (e.g., T cells) with IL-2, IL-15, and IL-21, (b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™, (c) contacting the population of cells (e.g., T cells) with a nucleic acid molecule (e.g., a DNA or RNA molecule) encoding the CAR, thereby providing a population of cells (e.g., T cells) comprising the nucleic acid molecule, and (d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells. In some embodiments, responsive to a decreased value for one, two, or all of the following in the population of cells (e.g., T cells): (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, (2) the percentage of CD28+ T cells among T cells, the percentage of CD28+CD4+ T cells among CD4+ T cells, or the percentage of CD28+CD8+ T cells among CD8+ T cells, and (3) the ratio of CD4+ T cells to CD8+ T cells, as compared to a reference value, e.g., a healthy donor reference value, performing the bead-free stimulation and cytokine (BFSC) process described in Example 1.

Also disclosed is a method of making a population of cells (e.g., T cells) that express a chimeric antigen receptor (CAR), the method comprising: responsive to an increased value of one, two, or all of the following in the population of cells (e.g., T cells): (4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, (5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and (6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, as compared to a reference value, e.g., a healthy donor reference value, performing: (a) contacting the population of cells (e.g., T cells) with IL-2 and one or both of: IL-15 and IL-21, e.g., contacting the population of cells (e.g., T cells) with IL-2, IL-15, and IL-21, (b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™, (c) contacting the population of cells (e.g., T cells) with a nucleic acid molecule (e.g., a DNA or RNA molecule) encoding the CAR, thereby providing a population of cells (e.g., T cells) comprising the nucleic acid molecule, and (d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells. In some embodiments, responsive to an increased value of one, two, or all of the following in the population of cells (e.g., T cells): (4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, (5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and (6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, as compared to a reference value, e.g., a healthy donor reference value, performing the bead-free stimulation and cytokine (BFSC) process described in Example 1.

In one aspect, this disclosure provides a method of making a population of cells (e.g., T cells) that express a chimeric antigen receptor (CAR), the method comprising: responsive to (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells (e.g., T cells) being lower than 8, 9, 10, 11, 12, 15, or 20%, e.g., 10%, (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells (e.g., T cells) being higher than 40, 45, 50, 55, or 60%, e.g. 50%, and/or (3) the percentage of CD28+ T cells among T cells, the percentage of CD28+CD4+ T cells among CD4+ T cells, or the percentage of CD28+CD8+ T cells among CD8+ T cells in the population of cells (e.g., T cells) being lower than 40, 45, 50, 55, or 60%, e.g. 50%, performing: (a) contacting the population of cells (e.g., T cells) with IL-2 and one or both of: IL-15 and IL-21, e.g., contacting the population of cells (e.g., T cells) with IL-2, IL-15, and IL-21, (b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™, (c) contacting the population of cells (e.g., T cells) with a nucleic acid molecule (e.g., a DNA or RNA molecule) encoding the CAR, thereby providing a population of cells (e.g., T cells) comprising the nucleic acid molecule, and (d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells. In some embodiments, responsive to (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells (e.g., T cells) being lower than 8, 9, 10, 11, 12, 15, or 20%, e.g., 10%, (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells (e.g., T cells) being higher than 40, 45, 50, 55, or 60%, e.g. 50%, and/or (3) the percentage of CD28+ T cells among T cells, the percentage of CD28+CD4+ T cells among CD4+ T cells, or the percentage of CD28+CD8+ T cells among CD8+ T cells in the population of cells (e.g., T cells) being lower than 40, 45, 50, 55, or 60%, e.g. 50%, performing the bead-free stimulation and cytokine (BFSC) process described in Example 1.

In one aspect, provided herein is a method of making a population of cells (e.g., T cells) that express a chimeric antigen receptor (CAR), the method comprising: responsive to (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells (e.g., T cells) being higher than 8, 9, 10, 11, 12, 15, or 20%, e.g., 10%, (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells (e.g., T cells) being lower than 40, 45, 50, 55, or 60%, e.g. 50%, and/or (3) the percentage of CD28+ T cells among T cells, the percentage of CD28+CD4+ T cells among CD4+ T cells, or the percentage of CD28+CD8+ T cells among CD8+ T cells in the population of cells (e.g., T cells) being higher than 40, 45, 50, 55, or 60%, e.g. 50%, performing the bead-free stimulation and cytokine (BFSC) process described in Example 1, or the Bead CAR T cell manufacturing process described in Example 1.

In one aspect, provided herein is a method of making a population of cells (e.g., T cells) that express a chimeric antigen receptor (CAR), the method comprising acquiring a value for one or more (e.g., 2, 3, 4, 5, or all) of the following from the population of cells (e.g., T cells): (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, (2) the percentage of CD28+ T cells among T cells, the percentage of CD28+CD4+ T cells among CD4+ T cells, or the percentage of CD28+CD8+ T cells among CD8+ T cells, (3) the ratio of CD4+ T cells to CD8+ T cells, (4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, (5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and (6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, wherein responsive to a decrease in the value of one, two, or all of (1)-(3), or responsive to an increase in the value of one, two, or all of (4)-(6), as compared to a reference value, e.g., a healthy donor reference value, performing: (a) contacting the population of cells (e.g., T cells) with IL-2 and one or both of: IL-15 and IL-21, e.g., contacting the population of cells (e.g., T cells) with IL-2, IL-15, and IL-21, (b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™, (c) contacting the population of cells (e.g., T cells) with a nucleic acid molecule (e.g., a DNA or RNA molecule) encoding the CAR, thereby providing a population of cells (e.g., T cells) comprising the nucleic acid molecule, and (d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells. In some embodiments, responsive to a decrease in the value of one, two, or all of (1)-(3), or responsive to an increase in the value of one, two, or all of (4)-(6), as compared to a reference value, e.g., a healthy donor reference value, performing the bead-free stimulation and cytokine (BFSC) process described in Example 1.

In one aspect, disclosed herein is a method of making a population of cells (e.g., T cells) that express a chimeric antigen receptor (CAR), the method comprising: acquiring a value for one or more (e.g., 2 or all) of the following from the population of cells (e.g., T cells): (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, and (3) the percentage of CD28+ T cells among T cells, the percentage of CD28+CD4+ T cells among CD4+ T cells, or the percentage of CD28+CD8+ T cells among CD8+ T cells, wherein: responsive to value (1) being lower than 8, 9, 10, 11, 12, 15, or 20%, e.g., 10%; value (2) being higher than 40, 45, 50, 55, or 60%, e.g. 50%; and/or value (3) being lower than 40, 45, 50, 55, or 60%, e.g. 50%, performing: (a) contacting the population of cells (e.g., T cells) with IL-2 and one or both of: IL-15 and IL-21, e.g., contacting the population of cells (e.g., T cells) with IL-2, IL-15, and IL-21, (b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™, (c) contacting the population of cells (e.g., T cells) with a nucleic acid molecule (e.g., a DNA or RNA molecule) encoding the CAR, thereby providing a population of cells (e.g., T cells) comprising the nucleic acid molecule, and (d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells. In some embodiments, responsive to value (1) being lower than 8, 9, 10, 11, 12, 15, or 20%, e.g., 10%; value (2) being higher than 40, 45, 50, 55, or 60%, e.g. 50%; and/or value (3) being lower than 40, 45, 50, 55, or 60%, e.g. 50%, performing the bead-free stimulation and cytokine (BFSC) process described in Example 1.

In one aspect, this invention provides a method of making a population of cells (e.g., T cells) that express a chimeric antigen receptor (CAR), the method comprising: acquiring a value for one or more (e.g., 2 or all) of the following from the population of cells (e.g., T cells): (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, and (3) the percentage of CD28+ T cells among T cells, the percentage of CD28+CD4+ T cells among CD4+ T cells, or the percentage of CD28+CD8+ T cells among CD8+ T cells, wherein: responsive to value (1) being higher than 8, 9, 10, 11, 12, 15, or 20%, e.g., 10%; value (2) being lower than 40, 45, 50, 55, or 60%, e.g. 50%; and/or value (3) being higher than 40, 45, 50, 55, or 60%, e.g. 50%, performing the bead-free stimulation and cytokine (BFSC) process described in Example 1, or the Bead CAR T cell manufacturing process described in Example 1.

In some embodiments of the aforementioned methods, the Bead CAR T cell manufacturing process described in Example 1 comprises: (a) providing an apheresis, e.g., leukapheresis, product, (b) isolating a population of cells (e.g., T cells) from the apheresis product and contacting the population of cells (e.g., T cells) using anti-CD3 and anti-CD28 antibodies coupled to Dynabeads, (c) contacting the population of cells (e.g., T cells) with IL-2, (d) contacting the population of cells (e.g., T cells) with a nucleic acid molecule (e.g., a DNA or RNA molecule) encoding the CAR, thereby providing a population of cells (e.g., T cells) comprising the nucleic acid molecule, and (e) expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells.

In some embodiments of the aforementioned methods, the CAR comprises an antigen binding domain, a transmembrane domain, and an intracellular signaling domain. In some embodiments, the antigen binding domain binds to an antigen chosen from: CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the antigen binding domain comprises a CDR, VH, VL, scFv or a CAR sequence disclosed herein.

In some embodiments, the antigen binding domain comprises a VH and a VL, wherein the VH and VL are connected by a linker. In some embodiments, the linker comprises the amino acid sequence of SEQ ID NO: 63 or 104.

In some embodiments, the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a transmembrane domain of CD8. In some embodiments, the transmembrane domain comprises the amino acid sequence of SEQ ID NO: 6, or an amino acid sequence having at least about 85%, 90%, 95%, or 99% sequence identity thereof. In some embodiments, the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the antigen binding domain is connected to the transmembrane domain by a hinge region. In some embodiments, the hinge region comprises the amino acid sequence of SEQ ID NO: 2, 3, or 4, or an amino acid sequence having at least about 85%, 90%, 95%, or 99% sequence identity thereof. In some embodiments, the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain. In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d. In some embodiments, the primary signaling domain comprises a functional signaling domain derived from CD3 zeta. In some embodiments, the primary signaling domain comprises the amino acid sequence of SEQ ID NO: 9 or 10, or an amino acid sequence having at least about 85%, 90%, 95%, or 99% sequence identity thereof. In some embodiments, the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain. In some embodiments, the costimulatory signaling domain comprises a functional signaling domain derived from a MHC class I molecule, a TNF receptor protein, an Immunoglobulin-like protein, a cytokine receptor, an integrin, a signalling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory signaling domain comprises a functional signaling domain derived from 4-1BB. In some embodiments, the costimulatory signaling domain comprises the amino acid sequence of SEQ ID NO: 7, or an amino acid sequence having at least about 85%, 90%, 95%, or 99% sequence identity thereof. In some embodiments, the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta. In some embodiments, the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof). In some embodiments, the intracellular signaling domain comprises the amino acid sequence of SEQ ID NO: 7 and the amino acid sequence of SEQ ID NO: 9 or 10.

In some embodiments, the CAR further comprises a leader sequence comprising the amino acid sequence of SEQ ID NO: 1.

In one aspect, this invention features a population of CAR-expressing cells (e.g., autologous or allogeneic CAR-expressing T cells or NK cells) made by any of the aforementioned methods or any other method disclosed herein. In one aspect, disclosed herein is a pharmaceutical composition comprising a population of CAR-expressing cells disclosed herein and a pharmaceutically acceptable carrier.

In one aspect, this invention features a method of increasing an immune response in a subject, comprising administering a population of CAR-expressing cells disclosed herein or a pharmaceutical composition disclosed herein to the subject, thereby increasing an immune response in the subject.

In one aspect, disclosed herein is a method of treating a cancer in a subject, comprising administering a population of CAR-expressing cells disclosed herein or a pharmaceutical composition disclosed herein to the subject, thereby treating the cancer in the subject. In one embodiment, the cancer is a solid cancer, e.g., chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof. In one embodiment, the cancer is a liquid cancer, e.g., chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In one embodiment, the method further comprises administering a second therapeutic agent to the subject.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. When one gene or protein references a plurality of sequence accession numbers, all of the sequence variants are encompassed.

In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a graph showing in vitro expansion of T cells isolated from a number of diffuse large B-cell lymphoma (DLBCL) patients. Viable cell numbers are plotted against days of expansion.

FIG. 2 is a graph outlining the Bead CAR T manufacturing process.

FIG. 3 is a graph comparing the impact of modified media (MM) which only contains IL-2, but not IL-15 or IL-21 (“MM Control”), and MM supplemented with IL-15 and IL-21 (“MM+IL15+IL21”) on T cell proliferation. Population doubling level (PDL) is plotted for each condition tested. Among the seven samples tested, six of them showed improved expansion, as measured by PDL, when the media was supplemented with IL-15 and IL-21.

FIG. 4 is a graph showing in vitro expansion of T cells isolated from a CLL patient (designated as sample “TR403”) using the bead-free stimulation and cytokine (BFSC) process (“TransAct+IL-2+IL-15+IL-21”) or bead control process (“Bead control”). Population doubling level (PDL) is plotted against days of expansion.

FIG. 5 is a graph showing similar results as in FIG. 4 for a CLL sample designated as “TR411.”

FIG. 6 is a graph showing similar results as in FIG. 4 for a CLL sample designated as “TR412.”

FIG. 7 is a pair of graphs showing the percentage of CD28+CD4+ T cells among CD4+ T cells (left) or the percentage of CD28+CD8+ T cells among CD8+ T cells (right) in the final CAR T product manufactured using the bead control process (“Bead control”) or the bead-free stimulation and cytokine (BFSC) process (“TransAct+IL-2+IL-15+IL-21”).

FIG. 8 is a pair of graphs showing the percentage of PD-1+CD4+ T cells among CD4+ T cells (left) or the percentage of PD-1+CD8+ T cells among CD8+ T cells (right) in the final CART product manufactured using the bead control process (“Bead control”), the bead-free control process (“TransAct”), or the bead-free stimulation and cytokine (BFSC) process (“TransAct+IL2+IL15+IL21”).

FIG. 9 is a pair of graphs showing the percentage of PD-1+LAG-3+CD4+ T cells among CD4+ T cells (left) or the percentage of PD-1+LAG-3+CD8+ T cells among CD8+ T cells (right) in the final CART product manufactured using the bead control process (“Bead control”), the bead-free control process (“TransAct”), or the bead-free stimulation and cytokine (BFSC) process (“TransAct+IL2+IL15+IL21”).

FIG. 10 is a graph showing in vitro expansion of T cells isolated from a CLL patient using the Bead CAR T manufacturing process (“Bead CAR T manufacturing process control”), a modified Bead CAR T manufacturing process in which the cells were incubated with IL-2, IL-15, and IL-21 (“Bead CAR T manufacturing process+cytokines”), a third manufacturing process in which T cells were negatively selected and stimulated with CD3/CD28 Dynabeads (“Neg. Selection+Beads”), and the bead-free stimulation and cytokine (BFSC) process (“TransAct+cytokines”). Population doubling level (PDL) is plotted against days of expansion.

FIG. 11 is a set of graphs showing in vitro expansion of T cells isolated from a healthy donor or a CLL patient using the Bead CAR T manufacturing process (“Bead CAR T manufacturing process”), a modified Bead CAR T manufacturing process in which the cells were incubated with IL-2, IL-15, and IL-21 (“Bead CAR T manufacturing process+cytokines”), and the bead-free stimulation and cytokine (BFSC) process (“TransAct+Cytokines”). Population doubling level (PDL) is plotted against days of expansion.

FIG. 12 is a graph outlining the bead-free stimulation and cytokine (BFSC) process.

FIG. 13 is a pair of graphs showing in vitro expansion of T cells (designated as samples “G71” (upper panel) and “F01” (lower panel)) using the Bead CAR T manufacturing process (“Bead CAR T manufacturing process control”) or the bead-free stimulation and cytokine (BFSC) process (“TransAct+IL-2+IL-15+IL-21”). Population doubling level (PDL) is plotted against days of expansion.

FIG. 14 is a pair of graphs showing in vitro expansion of T cells isolated from a CLL patient (upper panel) or a healthy donor (lower panel) using the Bead CAR T manufacturing process (“Bead CAR T manufacturing process control”) or the bead-free stimulation and cytokine (BFSC) process (“TransAct+IL-2+IL-15+IL-21”). Population doubling level (PDL) is plotted against days of expansion.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The compositions and methods of the present invention encompass polypeptides and nucleic acids having the sequences specified, or sequences substantially identical or similar thereto, e.g., sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term cytokine (for example, IL-2, IL-15, or IL-21) includes full length, a fragment or a variant, for example, a functional variant, of a naturally-occurring cytokine (including fragments and functional variants thereof having at least 10%, 30%, 50%, or 80% of the activity, e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R).

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 41BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a human or humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises an scFv.

As used herein, the term “binding domain” or “antibody molecule” (also referred to herein as “anti-target binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The terms “anti-tumor effect” and “anti-cancer effect” are used interchangeably and refer to a biological effect which can be manifested by various means, including but not limited to, e.g., a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, or amelioration of various physiological symptoms associated with the cancerous condition. An “anti-tumor effect” or “anti-cancer effect” can also be manifested by the ability of the peptides, polynucleotides, cells and antibodies of the invention in prevention of the occurrence of tumor or cancer in the first place.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation” in the context of stimulation by a stimulatory and/or costimulatory molecule refers to a response, e.g., a primary or secondary response, induced by binding of a stimulatory molecule (e.g., a TCR/CD3 complex) and/or a costimulatory molecule (e.g., CD28 or 4-1BB) with its cognate ligand thereby mediating a signal transduction event, such as, but not limited to, signal transduction via the TCR/CD3 complex. Stimulation can mediate altered expression of certain molecules and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of the invention, the intracellular signaling domain in any one or more CARS of the invention comprises an intracellular signaling sequence, e.g., a primary signaling sequence of CD3-zeta. The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In one embodiment, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In one embodiment, the “zeta stimulatory domain” or a “CD3-zeta stimulatory domain” is the sequence provided as SEQ ID NO: 9 or 10, or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions). In one embodiment, the “4-1BB costimulatory domain” is the sequence provided as SEQ ID NO: 7 or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen,” “tumor antigen,” “hyperproliferative disorder antigen,” and “antigen associated with a hyperproliferative disorder” interchangeably refer to antigens that are common to specific hyperproliferative disorders. In one embodiments, these terms refer to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (e.g., castrate-resistant or therapy-resistant prostate cancer, or metastatic prostate cancer), ovarian cancer, pancreatic cancer, and the like, or a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). In some embodiments, the CARs of the present invention include CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1 (SEQ ID NO: 41). For example, n=1, n=2, n=3. n=4, n=5 and n=6, n=7, n=8, n=9 and n=10 In one embodiment, the flexible polypeptide linkers include, but are not limited to, (Gly4 Ser)4 (SEQ ID NO: 27) or (Gly4 Ser)3 (SEQ ID NO: 28). In another embodiment, the linkers include multiple repeats of (Gly2Ser), (GlySer) or (Gly3Ser) (SEQ ID NO: 29). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g., RAD001.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In an embodiment, the low, immune enhancing, dose of mTOR inhibitor results in an increase in the number of naïve T cells. In an embodiment, the low, immune enhancing, dose of mTOR inhibitor results in one or more of the following:

an increase in the expression of one or more of the following markers: CD62L^{high}, CD127^{high}, CD27⁺, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62L^{high}, increased CD127^{high}, increased CD27⁺, decreased KLRG1, and increased BCL2;

wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (e.g., cancer) or the signs and symptoms of a disease such as cancer after a period of improvement or responsiveness, e.g., after prior treatment of a therapy, e.g., cancer therapy. The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “depletion” or “depleting”, as used interchangeably herein, refers to the decrease or reduction of the level or amount of a cell, a protein, or macromolecule in a sample after a process, e.g., a selection step, e.g., a negative selection, is performed. The depletion can be a complete or partial depletion of the cell, protein, or macromolecule. In an embodiment, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

As used herein, a “naïve T cell” refers to a T cell that is antigen-inexperienced. In some embodiments, naïve T cells may be differentiated, but have not yet encountered their cognate antigens in the peripheral. In some embodiments, naïve T cells are precursors of memory cells. In some embodiments, naïve T cells express CD45RA and CCR7, but not CD45RO. In some embodiments, naïve T cells may be characterized by expression of CD62L, CD27, CCR7, CD45RA, CD28, and CD127, and the absence of CD95, or CD45RO isoform. In some embodiments, naïve T cells express CD62L, IL-7 receptor-α, and CD132, but not CD25, CD44, CD69, or CD45RO.

The term “central memory T cells” refers to a subset of T cells that in humans are CD45RO positive and constitutively express CCR7 and CD62L. In some embodiments, central memory T cells express CD95. In some embodiments, central memory T cells express IL-2R, IL-7R and/or IL-15R.

As used herein, the term “bead” refers to a discrete particle with a solid surface, ranging in size from approximately 0.1 μm to several millimeters in diameter. Beads may be spherical (e.g., microspheres) or have an irregular shape. Beads may comprise a variety of materials including, but not limited to, paramagnetic materials, ceramic, plastic, glass, polystyrene, methylstyrene, acrylic polymers, titanium, latex, sepharose, cellulose, nylon and the like. In one embodiment, the beads are relatively uniform, about 4.5 μm in diameter, spherical, superparamagnetic polystyrene beads, e.g., coated, e.g., covalently coupled, with a mixture of antibodies against CD3 (e.g., CD3 epsilon) and CD28. In one embodiment, the beads are Dynabeads®. In one embodiment, both anti-CD3 and anti-CD28 antibodies are coupled to the same bead, mimicking stimulation of T cells by antigen presenting cells. The property of Dynabeads® and the use of Dynabeads® for cell isolation and expansion are well known in the art, e.g., see, Neurauter et al., Cell isolation and expansion using Dynabeads, Adv Biochem Eng Biotechnol. 2007; 106:41-73, herein incorporated by reference in its entirety.

As used herein, the term “nanomatrix” refers to a nanostructure comprising a matrix of mobile polymer chains. The nanomatrix is 1 to 500 nm, e.g., 10 to 200 nm, in size. In one embodiment, the matrix of mobile polymer chains is attached to one or more agonists which provide activation signals to T cells, e.g., agonist anti-CD3 and/or anti-CD28 antibodies. In one embodiment, the nanomatrix comprises a colloidal polymeric nanomatrix attached, e.g., covalently attached, to an agonist of one or more stimulatory molecules and/or an agonist of one or more costimulatory molecules. In one embodiment, the agonist of one or more stimulatory molecules is a CD3 agonist (e.g., an anti-CD3 agonistic antibody). In one embodiment, the agonist of one or more costimulatory molecules is a CD28 agonist (e.g., an anti-CD28 agonistic antibody). In one embodiment, the nanomatrix is characterized by the absence of a solid surface, e.g., as the attachment point for the agonists, such as anti-CD3 and/or anti-CD28 antibodies. In one embodiment, the nanomatrix is the nanomatrix disclosed in WO2014/048920A1 or as given in the MACS® GMP T Cell TransAct™ kit from Miltenyi Biotcc GmbH, herein incorporated by reference in their entirety. MACS® GMP T Cell TransAct™ consists of a colloidal polymeric nanomatrix covalently attached to humanized recombinant agonist antibodies against human CD3 and CD28.

Various aspects of the compositions and methods herein are described in further detail below. Additional definitions are set out throughout the specification.

### DESCRIPTION

Provided herein are methods of manufacturing immune cells (e.g., T cells or NK cells) engineered to express a CAR, e.g., a CAR described herein, compositions comprising such cells, and methods of using such cells for treating a disease, such as cancer, in a subject. In some embodiments, the immune cells (e.g., T cells) are isolated from apheresis, e.g., leukapheresis, material using negative selection, e.g., by reducing monocytes (e.g., CD14+ cells), B cells (e.g., CD19+ cells), and/or NK cells (e.g., CD56+ cells). In some embodiments, the immune cells (e.g., T cells) are incubated with IL-2, IL-15, and IL-21. In some embodiments, the immune cells (e.g., T cells) are contacted using an agent that stimulates a CD3/TCR complex (e.g., an agent that stimulates CD3, e.g., an anti-CD3 agonistic antibody) and an agent that stimulates a costimulatory molecule (e.g., an agent that stimulates CD28, e.g., an anti-CD28 agonistic antibody), optionally wherein the agent that stimulates a CD3/TCR complex or the agent that stimulates a costimulatory molecule does not comprise a bead. In one embodiment, the immune cells (e.g., T cells) are contacted with T Cell TransAct™.

Without wishing to be bound by theory, the manufacturing methods provided herein are able to expand immune cells (e.g., T cells) that would not have expanded or would have expanded only minimally (e.g., would have expanded for no more than 5, 6, 7, 8, or 9-fold over 8-11 days) using another CAR T cell manufacturing process, e.g., the Bead CAR T cell manufacturing process described herein. Some types of starting materials would especially benefit from the manufacturing methods described herein, e.g., leukapheresis material with a low amount of T cells, T cells with a low amount of naïve T cells and/or stem cell-like memory T cells (Tscm), T cells with a high amount of effector T cells (Teff) and/or effector memory T cells (Tem), T cells with low CD28 expression, T cells with a low CD4+ to CD8+ ratio, T cells with a high amount of senescent cells, or T cells with a high amount of exhausted cells.

Without wishing to be bound by theory, the manufacturing methods provided herein achieve greater expansion of immune cells (e.g., CAR-expressing T cells) than other CAR T cell manufacturing methods, e.g., the Bead CAR T cell manufacturing process described herein. Immune cells isolated from a patient, e.g., a cancer patient, may expand to a similar level as immune cells isolated from a healthy donor, using the manufacturing methods provided herein. In addition, CAR T cells produced using the manufacturing methods provided herein may show higher CD28 expression, lower PD-1 expression, lower LAG-3 expression, compared to CAR T cells produced using another manufacturing method, e.g., the Bead CAR T cell manufacturing process described herein.

### Bead-Free Stimulation and Cytokine (BFSC) Process

In one aspect, this invention features a CAR manufacturing process called bead-free stimulation and cytokine (BFSC) process. In some embodiments, a population of cells (e.g., T cells) is collected from an apheresis sample (e.g., a leukapheresis sample) from a subject. In some embodiment, the subject has cancer. In some embodiments, the apheresis sample (e.g., a leukapheresis sample) is collected from the subject and shipped as a frozen sample (e.g., a cryopreserved sample) to a cell manufacturing facility. The frozen apheresis sample is then thawed, and T cells (e.g., CD4+ T cells and/or CD8+ T cells) are selected from the apheresis sample using negative selection, e.g., by reducing the number of monocytes (e.g., CD14+ cells), B cells (e.g., CD19+ cells), and/or NK cells (e.g., CD56+ cells). The selected T cells (e.g., CD4+ T cells and/or CD8+ T cells) are then seeded for CAR T manufacturing using the bead-free stimulation and cytokine (BFSC) process described herein. In some embodiments, at the end of the manufacturing process, the CAR T cells are cryopreserved and later thawed and administered to the subject.

In one aspect, the present disclosure provides a method of making a population of cells (e.g., T cells) that express a chimeric antigen receptor (CAR), the method comprising: (i) contacting a population of cells (e.g., T cells) with IL-2 and one or both of: IL-15 and IL-21, and (ii) contacting the population of cells (e.g., T cells) with a nucleic acid molecule (e.g., a DNA or RNA molecule) encoding the CAR, thereby providing a population of cells (e.g., T cells) comprising the nucleic acid molecule.

In one embodiment, the nucleic acid molecule is on a viral vector. In one embodiment, the nucleic acid molecule is an RNA molecule on a viral vector. In one embodiment, step (ii) comprises transducing the population of cells (e.g., T cells) with a viral vector, e.g., a lentiviral vector or retroviral vector, comprising a nucleic acid molecule encoding the CAR. In one embodiment, the nucleic acid molecule is not on a viral vector. In one embodiment, step (ii) comprises electroporating the population of cells (e.g., T cells) with an RNA molecule encoding the CAR.

In one embodiment, the population of cells at the beginning of step (i) does not expand or expands for no more than 1, 2, 3, 4, 5, 6, 7, 8, 9, 10, 11, 12, 13, 14, or 15-fold over 4-11 days (e.g., 4, 5, 6, 7, 8, 9, 10, or 11 days) using the Bead CAR T cell manufacturing process described in Example 1.

In one embodiment, the percentage of naïve T cells among T cells in the population of cells at the beginning of step (i) is lower than 1, 2, 3, 4, 5, 6, 7, 8, 9, 10, 11, 12, 13, 14, 15, 20, 25, 30, 40, 50, or 60%, e.g., lower than 10%. In one embodiment, the percentage of CD4+ naïve T cells among CD4+ T cells in the population of cells at the beginning of step (i) is lower than 1, 2, 3, 4, 5, 6, 7, 8, 9, 10, 11, 12, 13, 14, 15, 20, 25, 30, 40, 50, or 60%, e.g., lower than 10%. In one embodiment, the percentage of CD8+ naïve T cells among CD8+ T cells in the population of cells at the beginning of step (i) is lower than 1, 2, 3, 4, 5, 6, 7, 8, 9, 10, 11, 12, 13, 14, 15, 20, 25, 30, 40, 50, or 60%, e.g., lower than 10%. In one embodiment, the percentage of naïve T cells among T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 15, 20, 25, 30, 35, 40, 45, 50, 55, 60, 65, 70, 75, 80, 85, or 90% lower than the corresponding value in a reference population of cells. In one embodiment, the percentage of CD4+ naïve T cells among CD4+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 15, 20, 25, 30, 35, 40, 45, 50, 55, 60, 65, 70, 75, 80, 85, or 90% lower than the corresponding value in a reference population of cells. In one embodiment, the percentage of CD8+ naïve T cells among CD8+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 15, 20, 25, 30, 35, 40, 45, 50, 55, 60, 65, 70, 75, 80, 85, or 90% lower than the corresponding value in a reference population of cells.

In one embodiment, the percentage of stem cell-like memory T cells (Tscm) among T cells in the population of cells at the beginning of step (i) is lower than 1, 2, 3, 4, 5, 6, 7, 8, 9, 10, 11, 12, 13, 14, 15, 20, 25, 30, 40, 50, or 60%, e.g., lower than 10%. In one embodiment, the percentage of CD4+ Tscm cells among CD4+ T cells in the population of cells at the beginning of step (i) is lower than 1, 2, 3, 4, 5, 6, 7, 8, 9, 10, 11, 12, 13, 14, 15, 20, 25, 30, 40, 50, or 60%, e.g., lower than 10%. In one embodiment, the percentage of CD8+ Tscm cells among CD8+ T cells in the population of cells at the beginning of step (i) is lower than 1, 2, 3, 4, 5, 6, 7, 8, 9, 10, 11, 12, 13, 14, 15, 20, 25, 30, 40, 50, or 60%, e.g., lower than 10%. In one embodiment, the percentage of Tscm among T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 15, 20, 25, 30, 35, 40, 45, 50, 55, 60, 65, 70, 75, 80, 85, or 90% lower than the corresponding value in a reference population of cells. In one embodiment, the percentage of CD4+ Tscm cells among CD4+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 15, 20, 25, 30, 35, 40, 45, 50, 55, 60, 65, 70, 75, 80, 85, or 90% lower than the corresponding value in a reference population of cells. In one embodiment, the percentage of CD8+ Tscm cells among CD8+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 15, 20, 25, 30, 35, 40, 45, 50, 55, 60, 65, 70, 75, 80, 85, or 90% lower than the corresponding value in a reference population of cells.

In one embodiment, the percentage of effector T cells (Teff) among T cells in the population of cells at the beginning of step (i) is higher than 20, 25, 30, 40, 50, 60, 70, 80, 90, or 95%, e.g., higher than 50%. In one embodiment, the percentage of CD4+ Teff cells among CD4+ T cells in the population of cells at the beginning of step (i) is higher than 20, 25, 30, 40, 50, 60, 70, 80, 90, or 95%, e.g., higher than 50%. In one embodiment, the percentage of CD8+Teff cells among CD8+ T cells in the population of cells at the beginning of step (i) is higher than 20, 25, 30, 40, 50, 60, 70, 80, 90, or 95%, e.g., higher than 50%. In one embodiment, the percentage of Teff cells among T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, or 1000% higher than the corresponding value in a reference population of cells. In one embodiment, the percentage of CD4+ Teff cells among CD4+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, or 1000% higher than the corresponding value in a reference population of cells. In one embodiment, the percentage of CD8+ Teff cells among CD8+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, or 1000% higher than the corresponding value in a reference population of cells.

In one embodiment, the percentage of effector memory T cells (Tem) among T cells in the population of cells at the beginning of step (i) is higher than 20, 25, 30, 40, 50, 60, 70, 80, 90, or 95%, e.g., higher than 50%. In one embodiment, the percentage of CD4+ Tem cells among CD4+ T cells in the population of cells at the beginning of step (i) is higher than 20, 25, 30, 40, 50, 60, 70, 80, 90, or 95%, e.g., higher than 50%. In one embodiment, the percentage of CD8+ Tem cells among CD8+ T cells in the population of cells at the beginning of step (i) is higher than 20, 25, 30, 40, 50, 60, 70, 80, 90, or 95%, e.g., higher than 50%. In one embodiment, the percentage of Tem cells among T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, or 1000% higher than the corresponding value in a reference population of cells. In one embodiment, the percentage of CD4+ Tem cells among CD4+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, or 1000% higher than the corresponding value in a reference population of cells. In one embodiment, the percentage of CD8+ Tem cells among CD8+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, or 1000% higher than the corresponding value in a reference population of cells.

In one embodiment, the percentage of CD28+CD4+ T cells among CD4+ T cells in the population of cells at the beginning of step (i) is no more than 20, 25, 30, 35, 40, 45, 50, 55, 60, 65, 70, 75, 80, 85, 90, or 95%, e.g., no more than 50%. In one embodiment, the percentage of CD28+CD8+ T cells among CD8+ T cells in the population of cells at the beginning of step (i) is no more than 10, 15, 20, 25, 30, 35, 40, 45, 50, 55, 60, 65, or 70%, e.g., no more than 50%. In one embodiment, the percentage of CD28+ T cells among T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 20, 30, 40, 50, 60, 70, 80, or 90% lower than the corresponding value in a reference population of cells. In one embodiment, the percentage of CD28+CD4+ T cells among CD4+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 20, 30, 40, 50, 60, 70, 80, or 90% lower than the corresponding value in a reference population of cells. In one embodiment, the percentage of CD28+CD8+ T cells among CD8+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 20, 30, 40, 50, 60, 70, 80, or 90% lower than the corresponding value in a reference population of cells.

In one embodiment, the ratio of CD4+ T cells to CD8+ T cells in the population of cells at the beginning of step (i) is lower than 0.5, 0.6, 0.7, 0.8, 0.9, 1, 1.1, 1.2, 1.3, 1.4, or 1.5. In one embodiment, the ratio of CD4+ T cells to CD8+ T cells in the population of cells at the beginning of step (i) is lower than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 10, 20, 30, 40, 50, 60, 70, 80, 90, or 99% lower than the corresponding value in a reference population of cells.

In one embodiment, the percentage of CD4+ senescent cells among CD4+ T cells in the population of cells at the beginning of step (i) is more than 2, 5, 10, 15, 20, 30, 40, 50, 60, 70, 80, 90 or 95%. In one embodiment, the percentage of CD28−CD27−CD57+CD4+ senescent cells among CD4+ T cells in the population of cells at the beginning of step (i) is more than 2, 5, 10, 15, 20, 30, 40, 50, 60, 70, 80, 90 or 95%. In one embodiment, the percentage of CD8+ senescent cells among CD8+ T cells in the population of cells at the beginning of step (i) is more than 2, 5, 10, 15, 20, 25, 30, 35, 40, 50, 60, 70, 80, 90 or 95%. In one embodiment, the percentage of CD28−CD27−CD57+CD8+ senescent cells among CD8+ T cells in the population of cells at the beginning of step (i) is more than 2, 5, 10, 15, 20, 25, 30, 35, 40, 50, 60, 70, 80, 90 or 95%. In one embodiment, the percentage of senescent T cells among T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000, or 9500% higher than the corresponding value in a reference population of cells. In one embodiment, the percentage of CD28−CD27−CD57+ senescent T cells among T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000, or 9500% higher than the corresponding value in a reference population of cells. In one embodiment, the percentage of CD4+ senescent cells among CD4+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000, or 9500% higher than the corresponding value in a reference population of cells. In one embodiment, the percentage of CD28−CD27−CD57+CD4+ senescent cells among CD4+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000, or 9500% higher than the corresponding value in a reference population of cells. In one embodiment, the percentage of CD8+ senescent cells among CD8+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000, or 9500% higher than the corresponding value in a reference population of cells. In one embodiment, the percentage of CD28−CD27−CD57+CD8+ senescent cells among CD8+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000, or 9500% higher than the corresponding value in a reference population of cells.

In one embodiment, the percentage of exhausted T cells (e.g., PD-1+, LAG3+, or PD-1+LAG3+ T cells) among T cells in the population of cells at the beginning of step (i) is higher than 5, 10, 15, 20, 25, 30, 35, 40, 45, 50, 55, 60, 65, 70, 80 or 90%. In one embodiment, the percentage of exhausted CD4+ T cells (e.g., PD-1+CD4+, LAG3+CD4+, or PD-1+LAG3+CD4+ T cells) among CD4+ T cells in the population of cells at the beginning of step (i) is higher than 5, 10, 15, 20, 25, 30, 35, 40, 45, 50, 55, 60, 65, 70, 80 or 90%. In one embodiment, the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than 5, 10, 15, 20, 25, 30, 35, 40, 45, 50, 55, 60, 65, 70, 80 or 90%. In one embodiment, the percentage of exhausted T cells (e.g., PD-1+, LAG3+, or PD-1+LAG3+ T cells) among T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000 or 9000% higher than the corresponding value in a reference population of cells. In one embodiment, the percentage of exhausted CD4+ T cells (e.g., PD-1+CD4+, LAG3+CD4+, or PD-1+LAG3+CD4+ T cells) among CD4+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000 or 9000% higher than the corresponding value in a reference population of cells. In one embodiment, the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than the corresponding value in a reference population of cells (e.g., a population of cells from a healthy donor, or a population of cells that expands more than 10, 15, 20, 25, 30, 35, 40, 45, or 50-fold over 4-11 days (e.g., 4, 5, 6, 7, 8, 9, 10, or 11 days) using the Bead CAR T cell manufacturing process described in Example 1), e.g., at least 20, 30, 40, 50, 60, 70, 80, 90, 100, 200, 300, 400, 500, 600, 700, 800, 900, 1000, 2000, 5000 or 9000% higher than the corresponding value in a reference population of cells.

In one embodiment, the population of cells (e.g., T cells) are contacted with IL-2, IL-15, and IL-21. In one embodiment, the population of cells (e.g., T cells) are contacted with IL-2 at about 10, 20, 30, 50, 60, 70, 80, 90, 100, 200, 300, 400, 500, 600, 700, or 800 U/mL. In one embodiment, the population of cells (e.g., T cells) are contacted with IL-15 at about 1, 2, 5, 10, 15, 20, 25, 30, 35, or 40 ng/mL. In one embodiment, the population of cells (e.g., T cells) are contacted with IL-21 at about 1, 2, 5, 10, 15, 20, 25, 30, 35, or 40 ng/mL. In one embodiment, about 0.5, 1, 1.5, or 2 days (e.g., 12, 15, 20, 24, 30, 35, 40, 45, or 48 hours) after the beginning of cytokine treatment described above, the population of cells (e.g., T cells) are contacted (e.g., transduced) with a nucleic acid molecule (e.g., a DNA or RNA molecule) encoding the CAR.

In one embodiment, the manufacturing method described herein further comprises (iii) contacting the population of cells (e.g., T cells) with an agent that stimulates a CD3/TCR complex (e.g., an anti-CD3 agonistic antibody) and/or an agent that stimulates a costimulatory molecule. In one embodiment, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In one embodiment, the agent that stimulates a costimulatory molecule is an agent that stimulates an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83. In one embodiment, the agent that stimulates a costimulatory molecule comprises an anti-CD28 agonistic antibody. In one embodiment, the agent that stimulates a CD3/TCR complex does not comprise a bead. In one embodiment, the agent that stimulates a costimulatory molecule does not comprise a bead.

In one embodiment, the population of cells (e.g., T cells) is contacted with a matrix, e.g., nanomatrix, coupled, e.g., covalently coupled, to an anti-CD3 antibody and/or an anti-CD28 antibody. In one embodiment, the agent that stimulates a CD3/TCR complex comprises an anti-CD3 antibody covalently attached to a colloidal polymeric nanomatrix. In one embodiment, the agent that stimulates a costimulatory molecule comprises an anti-CD28 antibody covalently attached to a colloidal polymeric nanomatrix. In one embodiment, the population of cells (e.g., T cells) is contacted with T Cell TransAct™.

In one embodiment, the agent that stimulates a CD3/TCR complex does not comprise hydrogel. In one embodiment, the agent that stimulates a costimulatory molecule does not comprise hydrogel. In one embodiment, the agent that stimulates a CD3/TCR complex does not comprise alginate. In one embodiment, the agent that stimulates a costimulatory molecule does not comprise alginate.

In one embodiment, the agent that stimulates a CD3/TCR complex comprises hydrogel. In one embodiment, the agent that stimulates a costimulatory molecule comprises hydrogel. In one embodiment, the agent that stimulates a CD3/TCR complex comprises alginate. In one embodiment, the agent that stimulates a costimulatory molecule comprises alginate. In one embodiment, the agent that stimulates a CD3/TCR complex or the agent that stimulates a costimulatory molecule comprises MagCloudz™ from Quad Technologies.

In one embodiment, the matrix comprises or consists of a polymeric, e.g., biodegradable or biocompatible inert material, e.g., which is non-toxic to cells. In one embodiment, the matrix is composed of hydrophilic polymer chains, which obtain maximal mobility in aqueous solution due to hydration of the chains. In one embodiment, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In one embodiment, the mobile matrix is a polymer of dextran.

In one embodiment, step (iii) is performed together with step (i). In one embodiment, step (iii) is performed no more than 1, 2, 3, 4, 5, 6, 7, 8, 9, or 10 hours prior to step (i). In one embodiment, step (iii) is performed no more than 1, 2, 3, 4, 5, 6, 7, 8, 9, or 10 hours after step (i).

In one embodiment, the population of cells at the beginning of step (i) is isolated from apheresis material using negative selection. In one embodiment, the population of cells at the beginning of step (i) is isolated from apheresis material by removing cells that are other than T cells, e.g., by reducing monocytes (e.g., CD14+ cells), B cells (e.g., CD19+ cells), and/or NK cells (e.g., CD56+ cells) from the apheresis material. In one embodiment, negative selection is performed using CliniMACS. In one embodiment, the apheresis material is leukapheresis material (e.g., fresh or frozen leukapheresis material). In one embodiment, the leukapheresis material comprises no more than 1, 5, 10, 15, 20, 25, 30, 35, or 40% T cells. In one embodiment, the percentage of T cells in the leukapheresis material is lower than the corresponding value in reference leukapheresis material (e.g., leukapheresis material from a healthy donor), e.g., at least 20, 30, 40, 50, 60, 70, 80, or 90% lower than the corresponding value in reference leukapheresis material.

In one embodiment, the population of cells at the beginning of step (i) is isolated from leukapheresis material from a subject having cancer. In one embodiment, the cancer is solid tumor. In one embodiment, the cancer is liquid tumor. In one embodiment, the cancer is chronic lymphoblastic leukemia (CLL). In one embodiment, the cancer is diffuse large B-cell lymphoma (DLBCL). In one embodiment, T cells from the subject failed to be manufactured into CAR-expressing cells using another CAR T manufacturing process, e.g., the Bead CAR T cell manufacturing process described herein.

In one embodiment, after the population of cells (e.g., T cells) are contacted with a nucleic acid molecule (e.g., a DNA or RNA molecule) encoding the CAR, the population of cells are expanded for at least 3, 4, 5, 6, 7, 8, 9, 10, 11, or 12 days in vitro, e.g., for about 4-10 days in vitro.

In one embodiment, the population of cells (e.g., T cells) are contacted with IL-2 and one or both of: IL-15 and IL-21 (e.g., all of IL-2, IL-15, and IL-21) are day 0, day 3, and subsequent days after day 3 (e.g., day 4, 5, 6, 7, 8, 9, or 10) in the manufacturing methods described herein (e.g., the BFSC process). In one embodiment, the population of cells (e.g., T cells) are contacted with IL-2 at about 10, 20, 30, 50, 60, 70, 80, 90, 100, 200, 300, 400, 500, 600, 700, or 800 U/mL. In one embodiment, the population of cells (e.g., T cells) are contacted with IL-15 at about 1, 2, 5, 10, 15, 20, 25, 30, 35, or 40 ng/mL. In one embodiment, the population of cells (e.g., T cells) are contacted with IL-21 at about 1, 2, 5, 10, 15, 20, 25, 30, 35, or 40 ng/mL.

In one aspect, featured herein is a method of making a population of cells (e.g., T cells) that express a chimeric antigen receptor (CAR), the method comprising:

(a) providing apheresis, e.g., leukapheresis, material, optionally wherein the apheresis, e.g., leukapheresis, material is cryopreserved after being isolated from a subject, and thawed prior to step (b),

(b) isolating a population of cells (e.g., T cells) from the apheresis, e.g., leukapheresis, material using negative selection, e.g., by reducing monocytes (e.g., CD14+ cells), B cells (e.g., CD19+ cells), and/or NK cells (e.g., CD56+ cells), from the apheresis, e.g., leukapheresis, material, e.g., using CliniMACS,

(c) contacting the population of cells (e.g., T cells) with IL-2 and one or both of: IL-15 and IL-21, e.g., contacting the population of cells (e.g., T cells) with IL-2, IL-15, and IL-21,

(d) contacting the population of cells (e.g., T cells) with an agent that stimulates a CD3/TCR complex and/or an agent that stimulates a costimulatory molecule, wherein the agent that stimulates a CD3/TCR complex or the agent that stimulates a costimulatory molecule does not comprise a bead, e.g., contacting the population of cells (e.g., T cells) with T Cell TransAct™,

(e) contacting the population of cells (e.g., T cells) with a nucleic acid molecule (e.g., a DNA or RNA molecule) encoding the CAR, thereby providing a population of cells (e.g., T cells) comprising the nucleic acid molecule, and

(f) expanding the population of cells comprising the nucleic acid molecule for at least 3, 4, 5, 6, 7, 8, 9, or 10 days in vitro, e.g., expanding the population of cells comprising the nucleic acid molecule for about 4-10 days, to produce a population of expanded cells.

Also featured in this disclosure are methods for evaluating sample suitability during CAR T manufacturing process. This invention discloses features of cells that are predictive or indicative of sample suitability for CAR T manufacturing, e.g., the percentage of naïve T cells (e.g., CD4+ or CD8+ naïve T cells), the percentage of stem cell-like memory T cells (Tscm) (e.g., CD4+ or CD8+ Tscm cells), the percentage of effector T cells (Teff) (e.g., CD4+ or CD8+ Teff cells), the percentage of effector memory T cells (Tem) (e.g., CD4+ or CD8+ Tem cells), the percentage of CD28+ T cells (e.g., CD4+ or CD8+CD28+ T cells), the ratio of CD4+ T cells to CD8+ T cells, the percentage of senescent T cells (e.g., CD28−CD27−CD57+ senescent T cells), and the percentage of exhausted T cells (e.g., PD-1+, LAG3+, or PD-1+LAG3+ T cells). In some embodiments, the percentage of naïve T cells and/or stem cell-like memory T cells (Tscm) among T cells being lower than 8, 9, 10, 11, 12, 13, 14, 15, 16, 17, 18, 19, or 20%, e.g., 10%, indicates or predicts decreased suitability for CAR manufacturing. In some embodiments, the percentage of effector T cells (Teff) and/or effector memory T cells (Tem) among T cells being higher than 40, 45, 50, 55, or 60%, e.g. 50%, indicates or predicts decreased suitability of the population of cells (e.g., T cells) for CAR manufacturing. In some embodiments, the percentage of CD28+ T cells among T cells, being lower than 30, 35, 40, 45, 50, 55, 60, 65, or 70%, e.g. 50%, indicates or predicts decreased suitability of the population of cells (e.g., T cells) for CAR manufacturing. In some embodiments, the ratio of CD4+ T cells to CD8+ T cells being lower than 0.5, 0.6, 0.7, 0.8, 0.9, 1, 1.1, 1.2, 1.3, 1.4, or 1.5 indicates or predicts decreased suitability of the population of cells (e.g., T cells) for CAR manufacturing. In some embodiments, the percentage of senescent T cells (e.g., CD28−CD27−CD57+ senescent T cells) among T cells being higher than 20, 25, 30, 35, 40, 45, 50, 55, 60, 65, 70, 75, 80, 85, 90, or 95% indicates or predicts decreased suitability of the population of cells (e.g., T cells) for CAR manufacturing. In some embodiments, the percentage of exhausted T cells (e.g., PD-1+, LAG3+, or PD-1+LAG3+ T cells) among T cells being higher than 10, 20, 30, 40, 50, 60, 70, 80, or 90% indicates or predicts decreased suitability of the population of cells (e.g., T cells) for CAR manufacturing.

Without wishing to be bound by theory, some starting materials particularly benefit from the CAR manufacturing methods described herein (e.g., the bead-free stimulation and cytokine (BFSC) process). Examples of such starting materials include a population of cells (e.g., T cells) with low levels of naïve T cells and/or stem cell-like memory T cells (Tscm), high levels of effector T cells (Teff) and/or effector memory T cells (Tem), and/or lower levels of CD28+ T cells. In one embodiment of such a starting material, the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells is lower than 8, 9, 10, 11, 12, 15, or 20%, e.g., 10%. In one embodiment of such a starting material, the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells is higher than 40, 45, 50, 55, or 60%, e.g. 50%. In one embodiment of such a starting material, the percentage of CD28+ T cells among T cells, the percentage of CD28+CD4+ T cells among CD4+ T cells, or the percentage of CD28+CD8+ T cells among CD8+ T cells is lower than 40, 45, 50, 55, or 60%, e.g. 50%. In one embodiment, after a starting material has been identified as fitting the description in this paragraph, the starting material is manufactured following the BFSC process described herein.

### Population of CAR-Expressing Cells Manufactured by the Processes Disclosed Herein

In another aspect, the disclosure features an immune effector cell (e.g., T cell or NK cell), e.g., made by any of the manufacturing methods described herein, engineered to express a CAR, wherein the engineered immune effector cell exhibits an antitumor property. In one embodiment, the CAR comprises an antigen binding domain, a transmembrane domain, and an intracellular signaling domain. An exemplary antigen is a cancer associated antigen described herein. In one aspect, the cell (e.g., T cell or NK cell) is transformed with the CAR and the CAR is expressed on the cell surface. In some embodiments, the cell (e.g., T cell or NK cell) is transduced with a viral vector encoding the CAR. In some embodiments, the viral vector is a retroviral vector. In some embodiments, the viral vector is a lentiviral vector. In some such embodiments, the cell may stably express the CAR. In another embodiment, the cell (e.g., T cell or NK cell) is transfected with a nucleic acid, e.g., mRNA, cDNA, or DNA, encoding a CAR. In some such embodiments, the cell may transiently express the CAR.

In some embodiments, provided herein is a population of cells (e.g., immune effector cells, e.g., T cells or NK cells) made by any of the manufacturing processes described herein (e.g., the bead-free stimulation and cytokine (BFSC) process described herein), engineered to express a CAR.

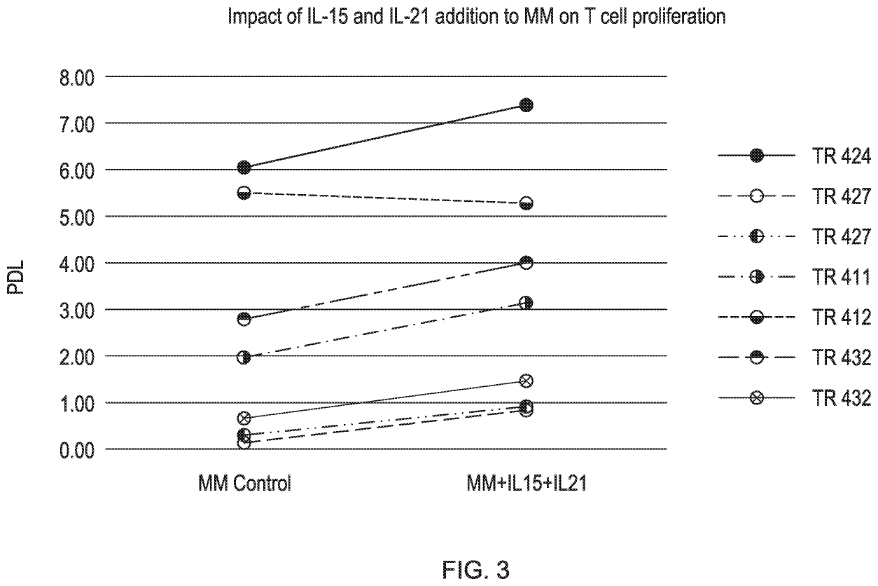

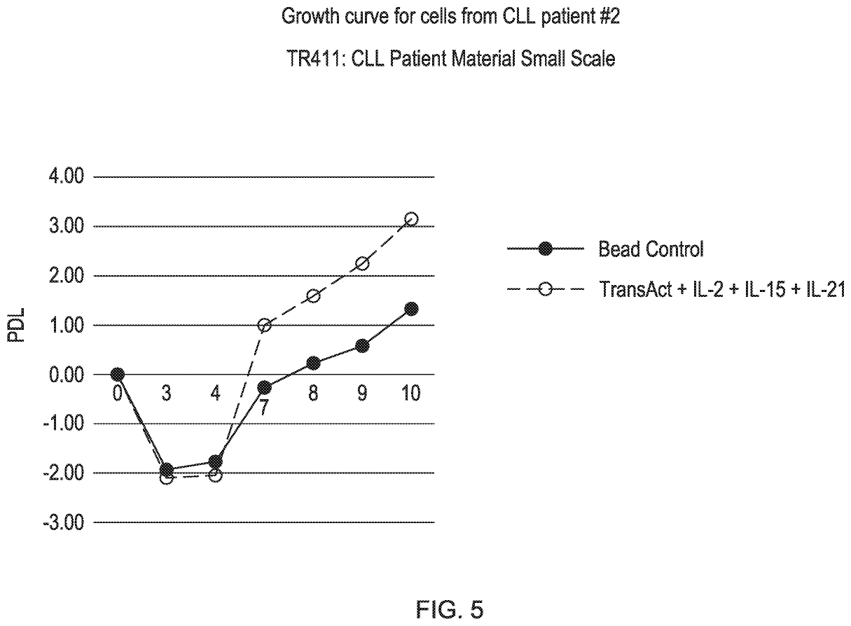

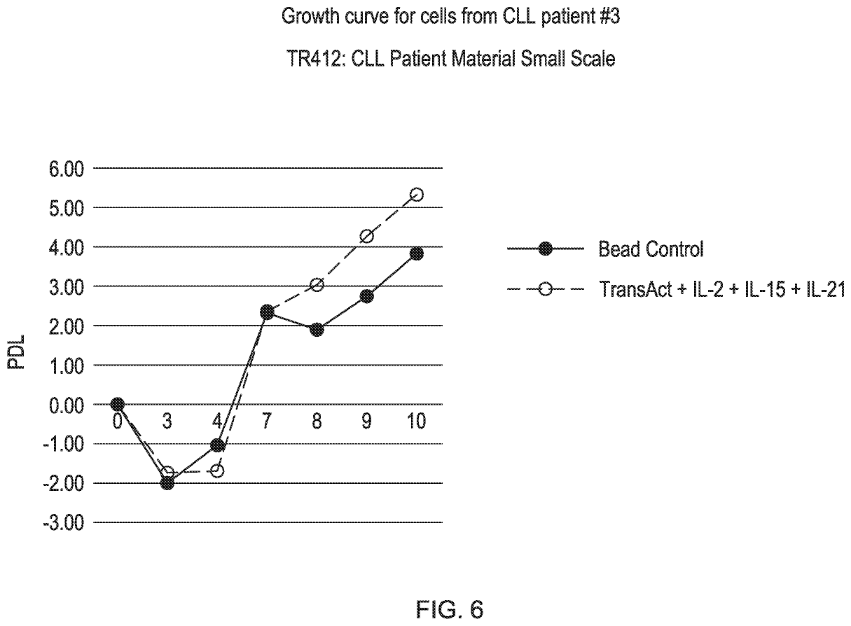

In one embodiment, CAR T cells manufactured using the methods described herein (e.g., a method in which the cells are contacted with IL-2, IL-15, and IL-21, or a method in which the cells are contacted with T Cell TransAct™, e.g., the BFSC process) show greater expansion (e.g., at least 5, 10, 15, 20, 25, 30, 40, 50, 100, 200, 300, 400, 500, 600, 700, 800, or 900% greater expansion) than CAR T cells manufactured using a different method (e.g., a method in which the cells are not contacted with IL-2, IL-15, or IL-21, or are contacted with IL-2 but not IL-15 or IL-21, or a method in which the cells are contacted with an agent that stimulates a CD3/TCR complex and comprises a bead, and/or an agent that stimulates a costimulatory molecule and comprises a bead, e.g., the Bead CAR T manufacturing process described herein), e.g., as measured by population doubling level (PDL), e.g., as assessed using methods described in Example 1 with respect to FIGS. 3-6, and 10.

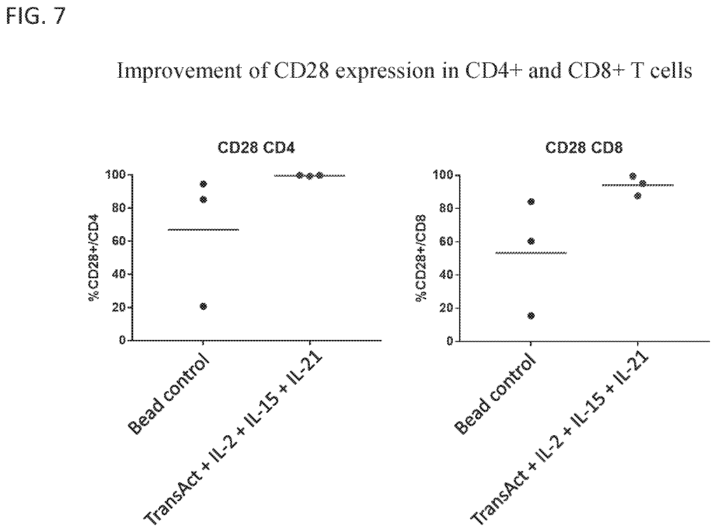

In one embodiment, CAR T cells manufactured using the methods described herein (e.g., a method in which the cells are contacted with IL-2, IL-15, and IL-21, or a method in which the cells are contacted with T Cell TransAct™, e.g., the BFSC process) show higher CD28 expression than CAR T cells manufactured using a different method (e.g., a method in which the cells are not contacted with IL-2, IL-15, or IL-21, or are contacted with IL-2 but not IL-15 or IL-21, or a method in which the cells are contacted with an agent that stimulates a CD3/TCR complex and comprises a bead, and/or an agent that stimulates a costimulatory molecule and comprises a bead, e.g., the Bead CAR T manufacturing process described herein), e.g., as measured by assessing the percentage of CD28+ T cells among T cells, the percentage of CD28+CD8+ T cells among CD8+ T cells, or the percentage of CD28+CD4+ T cells among CD4+ T cells, in CAR T cells, e.g., as assessed using methods described in Example 1 with respect to FIG. 7.

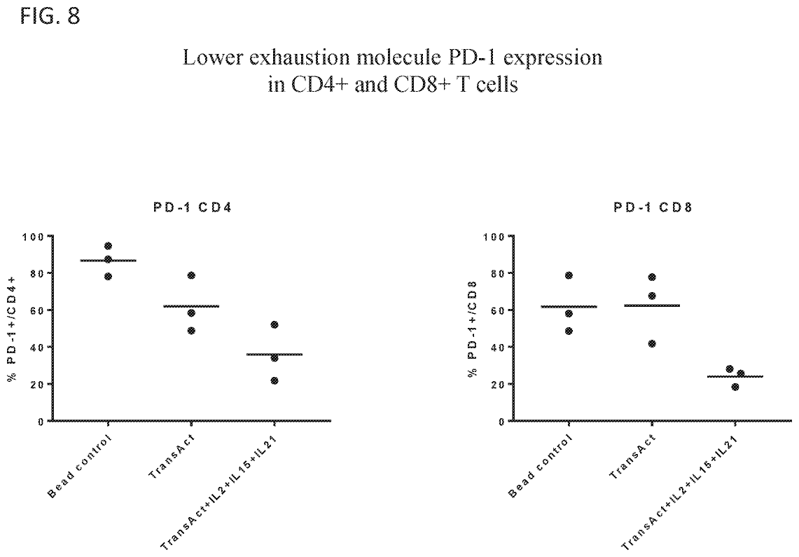

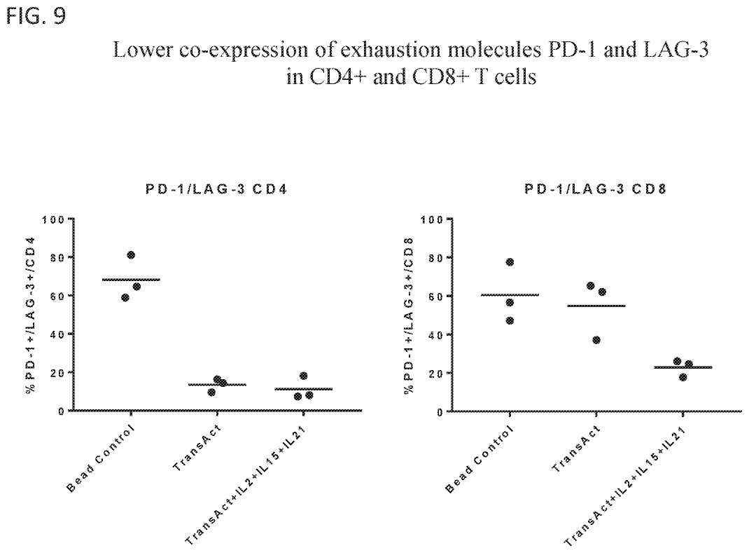

In one embodiment, CAR T cells manufactured using the methods described herein (e.g., a method in which the cells are contacted with IL-2, IL-15, and IL-21, or a method in which the cells are contacted with T Cell TransAct™, e.g., the BFSC process) show lower levels of exhausted T cells than CAR T cells manufactured using a different method (e.g., a method in which the cells are not contacted with IL-2, IL-15, or IL-21, or are contacted with IL-2 but not IL-15 or IL-21, or a method in which the cells are contacted with an agent that stimulates a CD3/TCR complex and comprises a bead, and/or an agent that stimulates a costimulatory molecule and comprises a bead, e.g., the Bead CAR T manufacturing process described herein), e.g., as measured by assessing the percentage of PD-1+CD8+ T cells among CD8+ T cells, the percentage of LAG3+CD8+ T cells among CD8+ T cells, the percentage of PD-1+LAG3+CD8+ T cells among CD8+ T cells, the percentage of PD-1+CD4+ T cells among CD4+ T cells, the percentage of LAG3+CD4+ T cells among CD4+ T cells, the percentage of PD-1+LAG3+CD4+ T cells among CD4+ T cells, in CART cells, e.g., as assessed using methods described in Example 1 with respect to FIGS. 8-9.

In some embodiments, using by the methods described herein (e.g., a method where the cells are contacted with IL-2, IL-15, and IL-21, or a method where the cells are contacted with T Cell TransAct™, e.g., the BFSC process), CAR T cells manufactured using cells isolated from a cancer patient can expand similarly as CAR T cells manufactured using cells isolated from a healthy donor.

### Pharmaceutical Composition

Furthermore, the present disclosure provides CAR-expressing cell compositions and their use in medicaments or methods for treating, among other diseases, cancer or any malignancy or autoimmune diseases involving cells or tissues which express a tumor antigen as described herein. In one aspect, provided herein are pharmaceutical compositions comprising a CAR-expressing cell, e.g., a plurality of CAR-expressing cells, made by a manufacturing process described herein (e.g., the BFSC process described herein), in combination with one or more pharmaceutically or physiologically acceptable carriers, diluents or excipients.

### Chimeric Antigen Receptor (CAR)

The present invention provides immune effector cells (e.g., T cells or NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that themselves are intracellular, however, fragments (peptides) of such antigens are presented on the surface of the cancer cells by MHC (major histocompatibility complex).

Accordingly, an immune effector cell, e.g., obtained by a method described herein, can be engineered to contain a CAR that targets one of the following cancer associated antigens (tumor antigens): CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

Sequences of non-limiting examples of various components that can be part of a CAR molecule described herein are listed in Table 1, where “aa” stands for amino acids, and “na” stands for nucleic acids that encode the corresponding peptide.

TABLE 1
Sequences of various components of CAR

| SEQ ID NO | Description | Sequence |
|---|---|---|
| SEQ ID NO: | EF-1 promoter | CGTGAGGCTCCGGTGCCCGTCAGTGGGCAGAGCGCACAT |
| 11 | (na) | CGCCCACAGTCCCCGAGAAGTTGGGGGGAGGGGTCGGC |
| | | AATTGAACCGGTGCCTAGAGAAGGTGGCGCGGGGTAAA |
| | | CTGGGAAAGTGATGTCGTGTACTGGCTCCGCCTTTTTCCC |
| | | GAGGGTGGGGGAGAACCGTATATAAGTGCAGTAGTCGC |
| | | CGTGAACGTTCTTTTTCGCAACGGGTTTGCCGCCAGAAC |
| | | ACAGGTAAGTGCCGTGTGTGGTTCCCGCGGGCCTGGCCT |
| | | CTTTACGGGTTATGGCCCTTGCGTGCCTTGAATTACTTCC |
| | | ACCTGGCTGCAGTACGTGATTCTTGATCCCGAGCTTCGG |
| | | GTTGGAAGTGGGTGGGAGAGTTCGAGGCCTTGCGCTTAA |
| | | GGAGCCCCTTCGCCTCGTGCTTGAGTTGAGGCCTGGCCT |
| | | GGGCGCTGGGGCCGCCGCGTGCGAATCTGGTGGCACCTT |
| | | CGCGCCTGTCTCGCTGCTTTCGATAAGTCTCTAGCCATTT |
| | | AAAATTTTTGATGACCTGCTGCGACGCTTTTTTTCTGGCA |
| | | AGATAGTCTTGTAAATGCGGGCCAAGATCTGCACACTGG |
| | | TATTTCGGTTTTTGGGGCCGCGGGCGGCGACGGGGCCCG |
| | | TGCGTCCCAGCGCACATGTTCGGCGAGGCGGGGCCTGCG |
| | | AGCGCGGCCACCGAGAATCGGACGGGGGTAGTCTCAAG |
| | | CTGGCCGGCCTGCTCTGGTGCCTGGCCTCGCGCCGCCGT |
| | | GTATCGCCCCGCCCTGGGCGGCAAGGCTGGCCCGGTCGG |
| | | CACCAGTTGCGTGAGCGGAAAGATGGCCGCTTCCCGGCC |
| | | CTGCTGCAGGGAGCTCAAAATGGAGGACGCGGCGCTCG |
| | | GGAGAGCGGGCGGGTGAGTCACCCACACAAAGGAAAAG |
| | | GGCCTTTCCGTCCTCAGCCGTCGCTTCATGTGACTCCACG |
| | | GAGTACCGGGCGCCGTCCAGGCACCTCGATTAGTTCTCG |
| | | AGCTTTTGGAGTACGTCGTCTTTAGGTTGGGGGGAGGGG |
| | | TTTTATGCGATGGAGTTTCCCCACACTGAGTGGGTGGAG |
| | | ACTGAAGTTAGGCCAGCTTGGCACTTGATGTAATTCTCC |
| | | TTGGAATTTGCCCTTTTTGAGTTTGGATCTTGGTTCATTC |
| | | TCAAGCCTCAGACAGTGGTTCAAAGTTTTTTTCTTCCATT |
| | | TCAGGTGTCGTGA |
| SEQ ID NO: | Leader (aa) | MALPVTALLLPLALLLHAARP |
| 1 | | |
| SEQ ID NO: | Leader (na) | ATGGCCCTGCCTGTGACAGCCCTGCTGCTGCCTCTGGCTC |
| 12 | | TGCTGCTGCATGCCGCTAGACCC |
| SEQ ID NO: | CD 8 hinge (aa) | TTTPAPRPPTPAPTIASQPLSLRPEACRPAAGGAVHTRGLDF |
| 2 | | ACD |
| SEQ ID NO: | CD8 hinge (na) | ACCACGACGCCAGCGCCGCGACCACCAACACCGGCGCC |
| 13 | | CACCATCGCGTCGCAGCCCCTGTCCCTGCGCCCAGAGGC |
| | | GTGCCGGCCAGCGGCGGGGGGCGCAGTGCACACGAGGG |
| | | GGCTGGACTTCGCCTGTGAT |
| SEQ ID NO: | Ig4 hinge (aa) | ESKYGPPCPPCPAPEFLGGPSVFLFPPKPKDTLMISRTPEVTC |
| 3 | | VVVDVSQEDPEVQFNWYVDGVEVHNAKTKPREEQFNSTY |
| | | RVVSVLTVLHQDWLNGKEYKCKVSNKGLPSSIEKTISKAK |
| | | GQPREPQVYTLPPSQEEMTKNQVSLTCLVKGFYPSDIAVEW |
| | | ESNGQPENNYKTTPPVLDSDGSFFLYSRLTVDKSRWQEGN |
| | | VFSCSVMHEALHNHYTQKSLSLSLGKM |
| SEQ ID NO: | Ig4 hinge (na) | GAGAGCAAGTACGGCCCTCCCTGCCCCCCTTGCCCTGCC |
| 14 | | CCCGAGTTCCTGGGCGGACCCAGCGTGTTCCTGTTCCCC |
| | | CCCAAGCCCAAGGACACCCTGATGATCAGCCGGACCCCC |
| | | GAGGTGACCTGTGTGGTGGTGGACGTGTCCCAGGAGGAC |
| | | CCCGAGGTCCAGTTCAACTGGTACGTGGACGGCGTGGAG |
| | | GTGCACAACGCCAAGACCAAGCCCCGGGAGGAGCAGTT |
| | | CAATAGCACCTACCGGGTGGTGTCCGTGCTGACCGTGCT |
| | | GCACCAGGACTGGCTGAACGGCAAGGAATACAAGTGTA |
| | | AGGTGTCCAACAAGGGCCTGCCCAGCAGCATCGAGAAA |
| | | ACCATCAGCAAGGCCAAGGGCCAGCCTCGGGAGCCCCA |
| | | GGTGTACACCCTGCCCCCTAGCCAAGAGGAGATGACCAA |
| | | GAACCAGGTGTCCCTGACCTGCCTGGTGAAGGGCTTCTA |
| | | CCCCAGCGACATCGCCGTGGAGTGGGAGAGCAACGGCC |
| | | AGCCCGAGAACAACTACAAGACCACCCCCCCTGTGCTGG |
| | | ACAGCGACGGCAGCTTCTTCCTGTACAGCCGGCTGACCG |
| | | TGGACAAGAGCCGGTGGCAGGAGGGCAACGTCTTTAGC |
| | | TGCTCCGTGATGCACGAGGCCCTGCACAACCACTACACC |
| | | CAGAAGAGCCTGAGCCTGTCCCTGGGCAAGATG |
| SEQ ID NO: | IgD hinge (aa) | RWPESPKAQASSVPTAQPQAEGSLAKATTAPATTRNTGRG |
| 4 | | GEEKKKEKEKEEQEERETKTPECPSHTQPLGVYLLTPAVQD |
| | | LWLRDKATFTCFVVGSDLKDAHLTWEVAGKVPTGGVEEG |
| | | LLERHSNGSQSQHSRLTLPRSLWNAGTSVTCTLNHPSLPPQ |
| | | RLMALREPAAQAPVKLSLNLLASSDPPEAASWLLCEVSGFS |
| | | PPNILLMWLEDQREVNTSGFAPARPPPQPGSTTFWAWSVLR |
| | | VPAPPSPQPATYTCVVSHEDSRTLLNASRSLEVSYVTDH |
| SEQ ID NO: | IgD hinge (na) | AGGTGGCCCGAAAGTCCCAAGGCCCAGGCATCTAGTGTT |
| 15 | | CCTACTGCACAGCCCCAGGCAGAAGGCAGCCTAGCCAA |
| | | AGCTACTACTGCACCTGCCACTACGCGCAATACTGGCCG |
| | | TGGCGGGGAGGAGAAGAAAAAGGAGAAAGAGAAAGAA |
| | | GAACAGGAAGAGAGGGAGACCAAGACCCCTGAATGTCC |
| | | ATCCCATACCCAGCCGCTGGGCGTCTATCTCTTGACTCCC |
| | | GCAGTACAGGACTTGTGGCTTAGAGATAAGGCCACCTTT |
| | | ACATGTTTCGTCGTGGGCTCTGACCTGAAGGATGCCCAT |
| | | TTGACTTGGGAGGTTGCCGGAAAGGTACCCACAGGGGG |
| | | GGTTGAGGAAGGGTTGCTGGAGCGCCATTCCAATGGCTC |
| | | TCAGAGCCAGCACTCAAGACTCACCCTTCCGAGATCCCT |
| | | GTGGAACGCCGGGACCTCTGTCACATGTACTCTAAATCA |
| | | TCCTAGCCTGCCCCCACAGCGTCTGATGGCCCTTAGAGA |
| | | GCCAGCCGCCCAGGCACCAGTTAAGCTTAGCCTGAATCT |
| | | GCTCGCCAGTAGTGATCCCCCAGAGGCCGCCAGCTGGCT |
| | | CTTATGCGAAGTGTCCGGCTTTAGCCCGCCCAACATCTT |
| | | GCTCATGTGGCTGGAGGACCAGCGAGAAGTGAACACCA |
| | | GCGGCTTCGCTCCAGCCCGGCCCCCACCCCAGCCGGGTT |
| | | CTACCACATTCTGGGCCTGGAGTGTCTTAAGGGTCCCAG |
| | | CACCACCTAGCCCCCAGCCAGCCACATACACCTGTGTTG |
| | | TGTCCCATGAAGATAGCAGGACCCTGCTAAATGCTTCTA |
| | | GGAGTCTGGAGGTTTCCTACGTGACTGACCATT |
| SEQ ID NO: | CD8 | IYIWAPLAGTCGVLLLSLVITLYC |
| 6 | Transmembrane | |
| | (aa) | |
| SEQ ID NO: | CD8 | ATCTACATCTGGGCGCCCTTGGCCGGGACTTGTGGGGTC |
| 17 | Transmembrane | CTTCTCCTGTCACTGGTTATCACCCTTTACTGC |
| | (na) | |
| SEQ ID NO: | 4-1BB intracellular | KRGRKKLLYIFKQPFMRPVQTTQEEDGCSCRFPEEEEGGCE |
| 7 | domain (aa) | L |
| SEQ ID NO: | 4-1BB intracellular | AAACGGGGCAGAAAGAAACTCCTGTATATATTCAAACA |
| 18 | domain (na) | ACCATTTATGAGACCAGTACAAACTACTCAAGAGGAAG |
| | | ATGGCTGTAGCTGCCGATTTCCAGAAGAAGAAGAAGGA |
| | | GGATGTGAACTG |
| SEQ ID NO: | CD27 (aa) | QRRKYRSNKGESPVEPAEPCRYSCPREEEGSTIPIQEDYRKP |
| 8 | | EPACSP |
| SEQ ID NO: | CD27 (na) | AGGAGTAAGAGGAGCAGGCTCCTGCACAGTGACTACAT |
| 19 | | GAACATGACTCCCCGCCGCCCCGGGCCCACCCGCAAGCA |
| | | TTACCAGCCCTATGCCCCACCACGCGACTTCGCAGCCTA |
| | | TCGCTCC |
| SEQ ID NO: | CD3-zeta (aa) | RVKFSRSADAPAYKQGQNQLYNELNLGRREEYDVLDKRR |
| 9 | (Q/K mutant) | GRDPEMGGKPRRKNPQEGLYNELQKDKMAEAYSEIGMKG |
| | | ERRRGKGHDGLYQGLSTATKDTYDALHMQALPPR |
| SEQ ID NO: | CD3-zeta (na) | AGAGTGAAGTTCAGCAGGAGCGCAGACGCCCCCGCGTA |
| 20 | (Q/K mutant) | CAAGCAGGGCCAGAACCAGCTCTATAACGAGCTCAATCT |
| | | AGGACGAAGAGAGGAGTACGATGTTTTGGACAAGAGAC |
| | | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ ID NO: | CD3-zeta (aa) | RVKFSRSADAPAYQQGQNQLYNELNLGRREEYDVLDKRR |
| 10 | (NCBI Reference | GRDPEMGGKPRRKNPQEGLYNELQKDKMAEAYSEIGMKG |
| | Sequence | ERRRGKGHDGLYQGLSTATKDTYDALHMQALPPR |
| | NM_000734.3) | |
| SEQ ID NO: | CD3-zeta (na) | AGAGTGAAGTTCAGCAGGAGCGCAGACGCCCCCGCGTA |
| 21 | (NCBI Reference | CCAGCAGGGCCAGAACCAGCTCTATAACGAGCTCAATCT |
| | Sequence | AGGACGAAGAGAGGAGTACGATGTTTTGGACAAGAGAC |
| | NM_000734.3) | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ ID NO: | CD28 Intracellular | RSKRSRLLHSDYMNMTPRRPGPTRKHYQPYAPPRDFAAYR |
| 36 | domain (amino acid | S |
| | sequence) | |
| SEQ ID NO: | CD28 Intracellular | AGGAGTAAGAGGAGCAGGCTCCTGCACAGTGACTACAT |
| 37 | domain (nucleotide | GAACATGACTCCCCGCCGCCCCGGGCCCACCCGCAAGCA |
| | sequence) | TTACCAGCCCTATGCCCCACCACGCGACTTCGCAGCCTA |
| | | TCGCTCC |
| SEQ ID NO: | ICOS Intracellular | T K K K Y S S S V H D P N G E Y M F M R A V N T A K K S R |
| 38 | domain (amino acid | L T D V T L |
| | sequence) | |
| SEQ ID NO: | ICOS Intracellular | ACAAAAAAGAAGTATTCATCCAGTGTGCACGACCCTAAC |
| 39 | domain (nucleotide | GGTGAATACATGTTCATGAGAGCAGTGAACACAGCCAA |
| | sequence) | AAAATCCAGACTCACAGATGTGACCCTA |
| SEQ ID NO: | GS hinge/linker | GGGGSGGGGS |
| 5 | (aa) | |
| SEQ ID NO: | GS hinge/linker | GGTGGCGGAGGTTCTGGAGGTGGAGGTTCC |
| 16 | (na) | |
| SEQ ID NO: | GS hinge/linker | GGTGGCGGAGGTTCTGGAGGTGGGGGTTCC |
| 40 | (na) | |
| SEQ ID NO: | linker | GGGGS |
| 25 | | |
| SEQ ID NO: | linker | (Gly-Gly-Gly-Gly-Ser)n, where n = 1-6, e.g., GGGGSGGGGS |
| 26 | | GGGGSGGGGS GGGGSGGGGS |
| SEQ ID NO: | linker | GGGGSGGGGSGGGGSGGGGS |
| 27 | | |
| SEQ ID NO: | linker | GGGGSGGGGSGGGGS |
| 28 | | |
| SEQ ID NO: | linker | GGGS |
| 29 | | |
| SEQ ID NO: | linker | (Gly-Gly-Gly-Ser)n where n is a positive integer equal to or |
| 41 | | greater than 1 |
| SEQ ID NO: | linker | (Gly-Gly-Gly-Ser)n, where n = 1-10, e.g., GGGSGGGSGG |
| 42 | | GSGGGSGGGS GGGSGGGSGG GSGGGSGGGS |
| SEQ ID NO: | linker | GSTSGSGKPGSGEGSTKG |
| 43 | | |
| SEQ ID NO: | polyA | (A)₅₀₀₀ |
| 30 | | This sequence may encompass 50-5000 adenines. |
| SEQ ID NO: | polyT | (T)₁₀₀ |
| 31 | | |
| SEQ ID NO: | polyT | (T)₅₀₀₀ |
| 32 | | This sequence may encompass 50-5000 thymines. |
| SEQ ID NO: | polyA | (A)₅₀₀₀ |
| 33 | | This sequence may encompass 100-5000 adenines. |
| SEQ ID NO: | polyA | (A)₄₀₀ |
| 34 | | This sequence may encompass 100-400 adenines. |
| SEQ ID NO: | polyA | (A)₂₀₀₀ |
| 35 | | This sequence may encompass 50-2000 adenines. |
| SEQ ID NO: | PD1 CAR (aa) | pgwfldspdrpwnpptfspallvvtegdnatftcsfsntsesfvlnwyrmspsnqtdklaaf |
| 22 | | pedrsqpgqdcrfrvtqlpngrdfhmsvvrarrndsgtylcgaislapkaqikeslraelrvt |
| | | erraevptahpspsprpagqfqtlvtttpaprpptpaptiasqplslrpeacrpaaggavhtrg |
| | | ldfacdiyiwaplagtcgvlllslvitlyckrgrkkllyifkqpfmrpvqttqeedgcscrfpe |
| | | eeeggcelrvkfsrsadapaykqgqnqlynelnlgrreeydvldkrrgrdpemggkprrk |
| | | npqeglynelqkdkmaeayseigmkgerrrgkghdglyqglstatkdtydalhmqalpp |
| | | r |
| SEQ ID NO: | PD-1 CAR (na) | atggccctccctgtcactgccctgcttctccccctcgcactcctgctccacgccgctagaccac |
| 23 | (PD1 ECD | ccggatggtttctggactctccggatcgcccgtggaatcccccaaccttctcaccggcactctt |
| | underlined) | ggttgtgactgagggcgataatgcgaccttcacgtgctcgttctccaacacctccgaatcattc |
| | | gtgctgaactggtaccgcatgagcccgtcaaaccagaccgacaagctcgccgcgtttccgga |
| | | agatcggtcgcaaccgggacaggattgtcggttccgcgtgactcaactgccgaatggcagag |
| | | acttccacatgagcgtggtccgcgctaggcgaaacgactccgggacctacctgtgcggagc |
| | | catctcgctggcgcctaaggcccaaatcaaagagagcttgagggccgaactgagagtgacc |
| | | gagcgcagagctgaggtgccaactgcacatccatccccatcgcctcggcctgcggggcagt |
| | | ttcagaccctggtcacgaccactccggcgccgcgcccaccgactccggccccaactatcgc |
| | | gagccagcccctgtcgctgaggccggaagcatgccgccctgccgccggaggtgctgtgcat |
| | | acccggggattggacttcgcatgcgacatctacatttgggctcctctcgccggaacttgtggcg |
| | | tgctccttctgtccctggtcatcaccctgtactgcaagcggggtcggaaaaagcttctgtacattt |
| | | tcaagcagcccttcatgaggcccgtgcaaaccacccaggaggaggacggttgctcctgccg |
| | | gttccccgaagaggaagaaggaggttgcgagctgcgcgtgaagttctcccggagcgccgac |
| | | gcccccgcctataagcagggccagaaccagctgtacaacgaactgaacctgggacggcgg |
| | | gaagagtacgatgtgctggacaagcggcgcggccgggaccccgaaatgggcgggaagcc |
| | | tagaagaaagaaccctcaggaaggcctgtataacgagctgcagaaggacaagatggccga |
| | | ggcctactccgaaattgggatgaagggagagcggcggaggggaaaggggcacgacggcc |
| | | tgtaccaaggactgtccaccgccaccaaggacacatacgatgccctgcacatgcaggccctt |
| | | ccccctcgc |
| SEQ ID NO: | PD-1 CAR (aa) | Malpvtalllplalllhaarppgwfldspdrpwnpptfspallvvtegdnatftcsfsntsesf |
| 24 | with signal | vlnwyrmspsnqtdklaafpedrsqpgqdcrfrvtqlpngrdfhmsvvrarrndsgtylc |
| | (PD1 ECD | gaislapkaqikeslraelrvterraevptahpspsprpagqfqtlvtttpaprpptpaptiasq |
| | underlined) | plslrpeacrpaaggavhtrgldfacdiyiwaplagtcgvlllslvitlyckrgrkkllyifkqp |
| | | fmrpvqttqeedgcscrfpeeeeggcelrvkfsrsadapaykqgqnqlynelnlgrreeyd |
| | | vldkttgrdpemggkprrknpqeglynelqkdkmaeayseigmkgeragkghdglyq |
| | | glstatkdtydalhmqalppr |

Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules, and various configurations for bispecific antibody molecules, are described in, e.g., paragraphs 455-458 of WO2015/142675, filed Mar. 13, 2015, which is incorporated by reference in its entirety.

In one aspect, the bispecific antibody molecule is characterized by a first immunoglobulin variable domain sequence, e.g., a scFv, which has binding specificity for CD19, e.g., comprises a scFv as described herein, or comprises the light chain CDRs and/or heavy chain CDRs from a scFv described herein, and a second immunoglobulin variable domain sequence that has binding specificity for a second epitope on a different antigen.

Chimeric TCR

In one aspect, the antibodies and antibody fragments of the present invention (e.g., CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, an scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, e.g., by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

Non-Antibody Scaffolds

In embodiments, the antigen binding domain comprises a non-antibody scaffold, e.g., a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

In an embodiment the antigen binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, wherein the CAR comprises an antigen binding domain (e.g., antibody or antibody fragment, TCR or TCR fragment) that binds specifically to a tumor antigen, e.g., an tumor antigen described herein, and an intracellular signaling domain. The intracellular signaling domain can comprise a costimulatory signaling domain and/or a primary signaling domain, e.g., a zeta chain. As described elsewhere, the methods described herein can include transducing a cell, e.g., from the population of T regulatory-depleted cells, with a nucleic acid encoding a CAR, e.g., a CAR described herein.

In specific aspects, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO:38, a transmembrane region such as provided in SEQ ID NO:6, an intracellular signalling domain that includes SEQ ID NO:7 or SEQ ID NO:16 and a CD3 zeta sequence that includes SEQ ID NO:9 or SEQ ID NO:10, e.g., wherein the domains are contiguous with and in the same reading frame to form a single fusion protein.

In one aspect, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:16. An exemplary CD3zeta domain sequence is provided as SEQ ID NO: 9 or SEQ ID NO:10.

In one aspect, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, e.g., a retroviral or lentiviral vector construct.

Nucleic acids encoding a CAR can also be introduced into the immune effector cell using, e.g., an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (e.g., described in the Examples, e.g., SEQ ID NO:35). RNA so produced can efficiently transfect different kinds of cells. In one embodiment, the template includes sequences for the CAR. In an embodiment, an RNA CAR vector is transduced into a cell, e.g., a T cell by electroporation.

### Antigen Binding Domain

In one aspect, a plurality of the immune effector cells, e.g., the population of T regulatory-depleted cells, include a nucleic acid encoding a CAR that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In one aspect, the portion of the CAR comprising the antigen binding domain comprises an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CD19 CAR

In some embodiments, the CAR-expressing cell described herein is a CD19 CAR-expressing cell (e.g., a cell expressing a CAR that binds to human CD19).

In one embodiment, the antigen binding domain of the CD19 CAR has the same or a similar binding specificity as the FMC63 scFv fragment described in Nicholson et al. Mol. Immun. 34 (16-17): 1157-1165 (1997). In one embodiment, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (e.g., a humanized antigen binding domain) according to Table 3 of WO2014/153270, incorporated herein by reference. WO2014/153270 also describes methods of assaying the binding and efficacy of various CAR constructs.

In one aspect, the parental murine scFv sequence is the CAR19 construct provided in PCT publication WO2012/079000 (incorporated herein by reference). In one embodiment, the anti-CD19 binding domain is a scFv described in WO2012/079000.

In one embodiment, the CAR molecule comprises the fusion polypeptide sequence provided as SEQ ID NO: 12 in PCT publication WO2012/079000, which provides an scFv fragment of murine origin that specifically binds to human CD19.

In one embodiment, the CD19 CAR comprises an amino acid sequence provided as SEQ ID NO: 12 in PCT publication WO2012/079000.

In one embodiment, the amino acid sequence is (SEQ ID NO: 123):

diqmtqttsslsaslgdrvtiscrasqdiskylnwyqqkpdgtvklliyhtsrlhsgvpsrfsgsgsgtdysltisnleqediat yfcqqgntlpytfgggtkleitggggsggggsggggsevklqesgpglvapsqslsvtctvsgvslpdygvswirqpprkglewlgv iwgsettyynsalksrltiikdnsksqvflkmnslqtddtaiyycakhyyyggsyamdywgqgtsvtvsstttpaprpptpaptiasq plslrpeacrpaaggavhtrgldfacdiyiwaplagtcgvlllslvitlyckrgrkkllyifkqpfmrpvqttqeedgcscrfpeeeeggc elrvkfsrsadapaykqgqnqlynelnlgrreeydvldkrrgrdpemggkprrknpqeglynelqkdkmaeayseigmkgerrrg kghdglyqglstatkdtydalhmqalppr, or a sequence substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In one embodiment, the CAR molecule is a humanized CD19 CAR comprising the amino acid sequence of (SEQ ID NO: 124):

EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSS

In one embodiment, the CAR molecule is a humanized CD19 CAR comprising the amino acid sequence of (SEQ ID NO: 125):

EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

Any known CD19 CAR, e.g., the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### BCMA CAR

In some embodiments, the CAR-expressing cell described herein is a BCMA CAR-expressing cell (e.g., a cell expressing a CAR that binds to human BCMA). Exemplary BCMA CARs can include sequences disclosed in Table 1 or 16 of WO2016/014565, incorporated herein by reference. The BCMA CAR construct can include an optional leader sequence; an optional hinge domain, e.g., a CD8 hinge domain; a transmembrane domain, e.g., a CD8 transmembrane domain; an intracellular domain, e.g., a 4-1BB intracellular domain; and a functional signaling domain, e.g., a CD3 zeta domain. In certain embodiments, the domains are contiguous and in the same reading frame to form a single fusion protein. In other embodiments, the domain are in separate polypeptides, e.g., as in an RCAR molecule as described herein.

In some embodiments, the BCMA CAR molecule includes one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (e.g., 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entireties. In some embodiments, additional exemplary BCMA CAR constructs are generated using the VH and VL sequences from PCT Publication WO2012/0163805 (the contents of which are hereby incorporated by reference in its entirety).

### CD20 CAR

In some embodiments, the CAR-expressing cell described herein is a CD20 CAR-expressing cell (e.g., a cell expressing a CAR that binds to human CD20). In some embodiments, the CD20 CAR-expressing cell includes an antigen binding domain according to WO2016/164731 and PCT/US2017/055627, incorporated herein by reference. Exemplary CD20-binding sequences or CD20 CAR sequences are disclosed in, e.g., Tables 1-5 of PCT/US2017/055627. In some embodiments, the CD20 CAR comprises a CDR, variable region, scFv, or full-length sequence of a CD20 CAR disclosed in PCT/US2017/055627 or WO2016/164731.

### CD22 CAR

In some embodiments, the CAR-expressing cell described herein is a CD22 CAR-expressing cell (e.g., a cell expressing a CAR that binds to human CD22). In some embodiments, the CD22 CAR-expressing cell includes an antigen binding domain according to WO2016/164731 and PCT/US2017/055627, incorporated herein by reference. Exemplary CD22-binding sequences or CD22 CAR sequences are disclosed in, e.g., Tables 6A, 6B, 7A, 7B, 7C, 8A, 8B, 9A, 9B, 10A, and 10B of WO2016/164731 and Tables 6-10 of PCT/US2017/055627. In some embodiments, the CD22 CAR sequences comprise a CDR, variable region, scFv or full-length sequence of a CD22 CAR disclosed in PCT/US2017/055627 or WO2016/164731.

### EGFR CAR

In some embodiments, the CAR-expressing cell described herein is an EGFR CAR-expressing cell (e.g., a cell expressing a CAR that binds to human EGFR). In some embodiments, the CAR-expressing cell described herein is an EGFRvIII CAR-expressing cell (e.g., a cell expressing a CAR that binds to human EGFRvIII). Exemplary EGFRvIII CARs can include sequences disclosed in WO2014/130657, e.g., Table 2 of WO2014/130657, incorporated herein by reference.

Exemplary EGFRvIII-binding sequences or EGFR CAR sequences may comprise a CDR, a variable region, an scFv, or a full-length CAR sequence of a EGFR CAR disclosed in WO2014/130657.

### Mesothelin CAR

In some embodiments, the CAR-expressing cell described herein is a mesothelin CAR-expressing cell (e.g., a cell expressing a CAR that binds to human mesothelin). Exemplary mesothelin CARs can include sequences disclosed in WO2015090230 and WO2017112741, e.g., Tables 2, 3, 4, and 5 of WO2017112741, incorporated herein by reference.

Other exemplary CARs

In other embodiments, the CAR-expressing cells can specifically bind to CD123, e.g., can include a CAR molecule (e.g., any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635. In other embodiments, the CAR-expressing cells can specifically bind to CD123, e.g., can include a CAR molecule (e.g., any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In other embodiments, the CAR-expressing cells can specifically bind to CLL-1, e.g., can include a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In an embodiment, the CAR molecule comprises a CD33 CAR described herein, e.g a CD33 CAR described in US2016/0096892A1, incorporated herein by reference. In embodiments, the CD33 CAR comprises an amino acid, or has a nucleotide sequence shown in US2016/0096892A1, incorporated herein by reference. In other embodiments, the CAR-expressing cells can specifically bind to CD33, e.g., can include a CAR molecule (e.g., any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In one embodiment, the antigen binding domain comprises one, two three (e.g., all three) heavy chain CDRs, HC CDR1, HC CDR2 and HC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (e.g., all three) light chain CDRs, LC CDR1, LC CDR2 and LC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In one embodiment, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1. In embodiments, the antigen binding domain targets CD19 and is described in US-2015-0283178-A1. In embodiments, the antigen binding domain targets CD123 and is described in US2014/0322212A1, US2016/0068601A1. In embodiments, the antigen binding domain targets CLL1 and is described in US2016/0051651A1. In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to GFR ALPHA-4, e.g., can include a CAR molecule, or an antigen binding domain according to Table 2 of WO2016/025880, incorporated herein by reference. The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In one embodiment, the antigen binding domain of any of the CAR molecules described herein (e.g., any of CD19, CD123, EGFRvIII, CD33, mesothelin, BCMA, and GFR ALPHA-4) comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antigen binding domain listed above. In one embodiment, the antigen binding domain comprises a heavy chain variable region and/or a variable light chain region of an antibody listed or described above.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed or described above.

In some embodiments, the tumor antigen is a tumor antigen described in International Application WO2015/142675, filed Mar. 13, 2015, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGalcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed or described above.

In one aspect, the anti-tumor antigen binding domain is a fragment, e.g., a single chain variable fragment (scFv). In one aspect, the anti-a cancer associate antigen as described herein binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1. In one embodiment, the linker can be (Gly₄Ser)₄ (SEQ ID NO: 27) or (Gly₄Ser)₃ (SEQ ID NO: 28). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the CAR-expressing cell, e.g., CART cell, surface. In a different aspect the amino acid sequence of the transmembrane domain may be modified or substituted so as to minimize interactions with the binding domains of the native binding partner present in the same CAR-expressing cell, e.g., CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of, e.g., the alpha, beta or zeta chain of T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO: 2. In one aspect, the transmembrane domain comprises (e.g., consists of) a transmembrane domain of SEQ ID NO: 6.

In one aspect, the hinge or spacer comprises an IgG4 hinge. For example, in one embodiment, the hinge or spacer comprises a hinge of SEQ ID NO: 3. In some embodiments, the hinge or spacer comprises a hinge encoded by the nucleotide sequence of SEQ ID NO: 14.

In one aspect, the hinge or spacer comprises an IgD hinge. For example, in one embodiment, the hinge or spacer comprises a hinge of the amino acid sequence of SEQ ID NO: 4. In some embodiments, the hinge or spacer comprises a hinge encoded by the nucleotide sequence of SEQ ID NO:15.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of SEQ ID NO: 5. In some embodiments, the linker is encoded by a nucleotide sequence of SEQ ID NO: 16.

In one aspect, the hinge or spacer comprises a KIR2DS2 hinge.

### Cytoplasmic Domain

The cytoplasmic domain or region of a CAR of the present invention includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Further examples of molecules containing a primary intracellular signaling domain that are of particular use in the invention include those of DAP10, DAP12, and CD32.

The intracellular signalling domain of the CAR can comprise the primary signalling domain, e.g., CD3-zeta signaling domain, by itself or it can be combined with any other desired intracellular signaling domain(s) useful in the context of a CAR of the invention. For example, the intracellular signaling domain of the CAR can comprise a primary signalling domain, e.g., CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 7. In one aspect, the signaling domain of CD3-zeta is a signaling domain of SEQ ID NO: 9 (mutant CD3zeta) or SEQ ID NO: 10 (wild type human CD3zeta).

In one aspect, the intracellular signaling domain is designed to comprise the signaling domain of CD3-zeta and the signaling domain of CD27. In one aspect, the signaling domain of CD27 comprises the amino acid sequence of SEQ ID NO: 8. In one aspect, the signalling domain of CD27 is encoded by the nucleic acid sequence of SEQ ID NO: 19.

In one aspect, the intracellular is designed to comprise the signaling domain of CD3-zeta and the signaling domain of CD28. In one aspect, the signaling domain of CD28 comprises the amino acid sequence of SEQ ID NO: 36. In one aspect, the signaling domain of CD28 is encoded by the nucleic acid sequence of SEQ ID NO: 37.

In one aspect, the intracellular is designed to comprise the signaling domain of CD3-zeta and the signaling domain of ICOS. In one aspect, the signaling domain of ICOS comprises the amino acid sequence of SEQ ID NO: 38. In one aspect, the signaling domain of ICOS is encoded by the nucleic acid sequence of SEQ ID NO: 39.
Co-Expression of CAR with Other Molecules or Agents

Co-Expression of a Second CAR

In one aspect, the CAR-expressing cell described herein can further comprise a second CAR, e.g., a second CAR that includes a different antigen binding domain, e.g., to the same target (e.g., CD19) or a different target (e.g., a target other than CD19, e.g., a target described herein). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In one embodiment, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In another embodiment, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In one embodiment, the CAR-expressing cell comprises an XCAR described herein and an inhibitory CAR. In one embodiment, the inhibitory CAR comprises an antigen binding domain that binds an antigen found on normal cells but not cancer cells, e.g., normal cells that also express X. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta).

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In an embodiment the antigen binding domain of one of the first and the second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, e.g., a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, e.g., a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, e.g., 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, e.g., 85%, 90%, 95%, 96%, 97%, 98% or 99% less than if both were scFv antigen binding domains.

Co-Expression of an Agent that Enhances CAR Activity

In another aspect, the CAR-expressing cell described herein can further express another agent, e.g., an agent that enhances the activity or fitness of a CAR-expressing cell.

For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In an embodiment, the agent that modulates or regulates, e.g., inhibits, T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In one embodiment, the PD1 CAR, when used in combinations with an XCAR described herein, improves the persistence of the T cell. In one embodiment, the CAR is a PD1 CAR comprising the extracellular domain of PD1 indicated as underlined in SEQ ID NO: 24. In one embodiment, the PD1 CAR comprises the amino acid sequence of SEQ ID NO: 24.

In one embodiment, the PD1 CAR comprises the amino acid sequence of SEQ ID NO: 22.

In one embodiment, the agent comprises a nucleic acid sequence encoding the PD1 CAR, e.g., the PD1 CAR described herein. In one embodiment, the nucleic acid sequence for the PD1 CAR is provided as SEQ ID NO: 23, with the PD1 ECD underlined.

In another example, in one embodiment, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In an embodiment, the costimulatory molecule ligand is 4-1BBL. In an embodiment, the costimulatory ligand is CD80 or CD86. In an embodiment, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, e.g., CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., CAR-Tx) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention also provides an immune effector cell, e.g., made by a method described herein, that includes a nucleic acid molecules encoding one or more CAR constructs described herein. In one aspect, the nucleic acid molecule is provided as a messenger RNA transcript. In one aspect, the nucleic acid molecule is provided as a DNA construct.

The nucleic acid molecules described herein can be a DNA molecule, an RNA molecule, or a combination thereof. In one embodiment, the nucleic acid molecule is an mRNA encoding a CAR polypeptide as described herein. In other embodiments, the nucleic acid molecule is a vector that includes any of the aforesaid nucleic acid molecules.

In one aspect, the antigen binding domain of a CAR of the invention (e.g., a scFv) is encoded by a nucleic acid molecule whose sequence has been codon optimized for expression in a mammalian cell. In one aspect, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Accordingly, in one aspect, an immune effector cell, e.g., made by a method described herein, includes a nucleic acid molecule encoding a chimeric antigen receptor (CAR), wherein the CAR comprises an antigen binding domain that binds to a tumor antigen described herein, a transmembrane domain (e.g., a transmembrane domain described herein), and an intracellular signaling domain (e.g., an intracellular signaling domain described herein) comprising a stimulatory domain, e.g., a costimulatory signaling domain (e.g., a costimulatory signaling domain described herein) and/or a primary signaling domain (e.g., a primary signaling domain described herein, e.g., a zeta chain described herein).

The present invention also provides vectors in which a nucleic acid molecule encoding a CAR, e.g., a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR is an adenoviral vector (A5/35). In another embodiment, the expression of nucleic acids encoding CARs can be accomplished using of transposons such as sleeping beauty, crisper, CAS9, and zinc finger nucleases. See below June et al. 2009 Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from nucleic acid molecules cloned into a lentiviral vector. See, e.g., Milone et al., Mol. Ther. 17(8): 1453-1464 (2009). In one aspect, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired.

The nucleotide sequences of exemplary PGK promoters are provided below.

WT PGK Promoter:

(SEQ ID NO: 109)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT

Exemplary Truncated PGK Promoters:

PGK100:
(SEQ ID NO: 110)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 111)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 112)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 113)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, e.g., a CAR described herein, e.g., a CD19 CAR, and a second CAR, e.g., an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, e.g., mammalian, bacterial, yeast, or insect cell by any method, e.g., one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, e.g., a caspase fused to a dimerization domain (see, e.g., Di Stasa et al., N Engl. J. Med. 2011 Nov. 3; 365(18):1673-1683), can be used as a safety switch in the CAR therapy of the instant invention. In one embodiment, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI¾β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab, as described in the Examples herein.

In other embodiments, an RCAR comprises a set of polypeptides, typically two in the simplest embodiments, in which the components of a standard CAR described herein, e.g., an antigen binding domain and an intracellular signalling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signalling domain. In one embodiment, a CAR of the present invention utilizes a dimerization switch as those described in, e.g., WO2014127261, which is incorporated by reference herein. Additional description and exemplary configurations of such regulatable CARs are provided herein and in, e.g., paragraphs 527-551 of International Publication No. WO 2015/090229 filed Mar. 13, 2015, which is incorporated by reference in its entirety. In some embodiments, an RCAR involves a switch domain, e.g., a FKBP switch domain, as set out SEQ ID NO: 114, or comprise a fragment of FKBP having the ability to bind with FRB, e.g., as set out in SEQ ID NO: 115. In some embodiments, the RCAR involves a switch domain comprising a FRB sequence, e.g., as set out in SEQ ID NO: 116, or a mutant FRB sequence, e.g., as set out in any of SEQ ID Nos. 117-122.

(SEQ ID NO: 114)
DVPDYASLGGPSSPKKKRKVSRGVQVETISPGDGRTFPKRGQTCVVHYTG
MLEDGKKFDSSRDRNKPFKFMLGKQEVIRGWEEGVAQMSVGQRAKLTISP
DYAYGATGHPGIIPPHATLVFDVELLKLETSY
(SEQ ID NO: 115)
VQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDV
ELLKLETS
(SEQ ID NO: 116)
ILWHEMWHEGLEEASRLYFGERNVKGMFEVLEPLHAMMERGPQTLKETSF
NQAYGRDLMEAQEWCRKYMKSGNVKDLTQAWDLYYHVFRRISK

TABLE 2
Exemplary mutant FRB having increased affinity for a dimerization molecule.

| | | SEQ |
|---|---|---|
| | | ID |
| FRB mutant | Amino Acid Sequence | NO: |
| E20321 mutant | ILWHEMWHEGLIEASRLYFGERNVKGMFEVLEPLHAMMERGPQTLKETSFNQAYG | 117 |
| | RDLMEAQEWCRKYMKSGNVKDLTQAWDLYYHVFRRISKTS | |
| E2032L mutant | ILWHEMWHEGLLEASRLYFGERNVKGMFEVLEPLHAMMERGPQTLKETSFNQAYG | 118 |
| | RDLMEAQEWCRKYMKSGNVKDLTQAWDLYYHVFRRISKTS | |
| T2098L mutant | ILWHEMWHEGLEEASRLYFGERNVKGMFEVLEPLHAMMERGPQTLKETSFNQAYG | 119 |
| | RDLMEAQEWCRKYMKSGNVKDLLQAWDLYYHVFRRISKTS | |
| E2032, T2098 | ILWHEMWHEGLXEASRLYFGERNVKGMFEVLEPLHAMMERGPQTLKETSFNQAYG | 120 |
| mutant | RDLMEAQEWCRKYMKSGNVKDLXQAWDLYYHVFRRISKTS | |
| E2032L, T2098L | ILWHEMWHEGLIEASRLYFGERNVKGMFEVLEPLHAMMERGPQTLKETSFNQAYG | 121 |
| mutant | RDLMEAQEWCRKYMKSGNVKDLLQAWDLYYHVFRRISKTS | |
| E2032L, T2098L | ILWHEMWHEGLLEASRLYFGERNVKGMFEVLEPLHAMMERGPQTLKETSFNQAYG | 122 |
| mutant | RDLMEAQEWCRKYMKSGNVKDLLQAWDLYYHVFRRISKTS | |

### RNA Transfection

Disclosed herein are methods for producing an in vitro transcribed RNA CAR. RNA CAR and methods of using the same are described, e.g., in paragraphs 553-570 of in International Application WO2015/142675, filed Mar. 13, 2015, which is herein incorporated by reference in its entirety.

An immune effector cell can include a CAR encoded by a messenger RNA (mRNA). In one aspect, the mRNA encoding a CAR described herein is introduced into an immune effector cell, e.g., made by a method described herein, for production of a CAR-expressing cell.

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (e.g., a hinge region described herein), a transmembrane domain (e.g., a transmembrane domain described herein such as a transmembrane domain of CD8a); and a cytoplasmic region that includes an intracellular signaling domain, e.g., an intracellular signaling domain described herein, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In an embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 31) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (e.g., SEQ ID NO: 33).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In an embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams. Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tcl/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

Methods of Manufacture/Production

In some embodiments, the methods disclosed herein further include administering a T cell depleting agent after treatment with the cell (e.g., an immune effector cell as described herein), thereby reducing (e.g., depleting) the CAR-expressing cells (e.g., the CD19CAR-expressing cells). Such T cell depleting agents can be used to effectively deplete CAR-expressing cells (e.g., CD19CAR-expressing cells) to mitigate toxicity. In some embodiments, the CAR-expressing cells were manufactured according to a method herein, e.g., assayed (e.g., before or after transfection or transduction) according to a method herein.

In some embodiments, the T cell depleting agent is administered one, two, three, four, or five weeks after administration of the cell, e.g., the population of immune effector cells, described herein.

In one embodiment, the T cell depleting agent is an agent that depletes CAR-expressing cells, e.g., by inducing antibody dependent cell-mediated cytotoxicity (ADCC) and/or complement-induced cell death. For example, CAR-expressing cells described herein may also express an antigen (e.g., a target antigen) that is recognized by molecules capable of inducing cell death, e.g., ADCC or complement-induced cell death. For example, CAR expressing cells described herein may also express a target protein (e.g., a receptor) capable of being targeted by an antibody or antibody fragment. Examples of such target proteins include, but are not limited to, EpCAM, VEGFR, integrins (e.g., integrins αvβ3, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/1gE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

In some embodiments, the CAR expressing cell co-expresses the CAR and the target protein, e.g., naturally expresses the target protein or is engineered to express the target protein. For example, the cell, e.g., the population of immune effector cells, can include a nucleic acid (e.g., vector) comprising the CAR nucleic acid (e.g., a CAR nucleic acid as described herein) and a nucleic acid encoding the target protein.

In one embodiment, the T cell depleting agent is a CD52 inhibitor, e.g., an anti-CD52 antibody molecule, e.g., alemtuzumab.

In other embodiments, the cell, e.g., the population of immune effector cells, expresses a CAR molecule as described herein (e.g., CD19CAR) and the target protein recognized by the T cell depleting agent. In one embodiment, the target protein is CD20. In embodiments where the target protein is CD20, the T cell depleting agent is an anti-CD20 antibody, e.g., rituximab.

In further embodiments of any of the aforesaid methods, the methods further include transplanting a cell, e.g., a hematopoietic stem cell, or a bone marrow, into the mammal.

In another aspect, the invention features a method of conditioning a mammal prior to cell transplantation. The method includes administering to the mammal an effective amount of the cell comprising a CAR nucleic acid or polypeptide, e.g., a CD19 CAR nucleic acid or polypeptide. In some embodiments, the cell transplantation is a stem cell transplantation, e.g., a hematopoietic stem cell transplantation, or a bone marrow transplantation. In other embodiments, conditioning a subject prior to cell transplantation includes reducing the number of target-expressing cells in a subject, e.g., CD19-expressing normal cells or CD19-expressing cancer cells.

### Elutriation

In one aspect, the methods described herein feature an elutriation method that removes unwanted cells, e.g., monocytes and blasts, thereby resulting in an improved enrichment of desired immune effector cells suitable for CAR expression. In one embodiment, the elutriation method described herein is optimized for the enrichment of desired immune effector cells suitable for CAR expression from a previously frozen sample, e.g., a thawed sample. In one embodiment, the elutriation method described herein provides a preparation of cells with improved purity as compared to a preparation of cells collected from the elutriation protocols known in the art. In an embodiment, the elutriation method described herein includes using an optimized viscosity of the starting sample, e.g., cell sample, e.g., thawed cell sample, by dilution with certain isotonic solutions (e.g., PBS), and using an optimized combination of flow rates and collection volume for each fraction collected by an elutriation device. Exemplary elutriation methods that could be applied in the present invention are described on pages 48-51 of WO 2017/117112, herein incorporated by reference in its entirety.

### Density Gradient Centrifugation

Manufacturing of adoptive cell therapeutic product requires processing the desired cells, e.g., immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, e.g., as described in the Examples herein. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, e.g., T cells, dehydrate during cryopreservation to become denser than fresh cells. Without wishing to be bound by theory, it is also believed that immune effector cells, e.g., T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, e.g., 1.077 g/mL.

In one embodiment, the density gradient centrifugation method described herein includes the use of a density gradient medium comprising iodixanol. In one embodiment, the density gradient medium comprises about 60% iodixanol in water.

In one embodiment, the density gradient centrifugation method described herein includes the use of a density gradient medium having a density greater than Ficoll. In one embodiment, the density gradient centrifugation method described herein includes the use of a density gradient medium having a density greater than 1.077 g/mL, e.g., greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In one embodiment, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In one embodiment, the selection comprises a positive selection, e.g., selection for the desired immune effector cells. In another embodiment, the selection comprises a negative selection, e.g., selection for unwanted cells, e.g., removal of unwanted cells. In embodiments, the positive or negative selection methods described herein are performed under flow conditions, e.g., by using a flow-through device, e.g., a flow-through device described herein. Exemplary positive and negative selections are described on pages 53-57 of WO 2017/117112, herein incorporated by reference in its entirety. Selection methods can be performed under flow conditions, e.g., by using a flow-through device, also referred to as a cell processing system, to further enrich a preparation of cells for desired immune effector cells, e.g., T cells, suitable for CAR expression. Exemplary flow-through devices are described on pages 57-70 of WO 2017/117112, herein incorporated by reference in its entirety. Exemplary cell separation and debeading methods are described on pages 70-78 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS Plus or CliniMACS Prodigy) or positive T cell selection with a combination of CD4 and CD8 Miltenyi beads and column technology (CliniMACS Plus or CliniMACS Prodigy) can be used. Alternatively, column-free technology with releasable CD3 beads (GE Healthcare) can be used.

In addition, bead-free technologies such as ThermoGenesis X-series devices can be utilized as well.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion. For instance, paramagnetic particles coated with anti-CD3 and/or anti CD28 may be used to select T cells for expansion or for introduction of a nucleic acid encoding a chimeric antigen receptor (CAR) or other protein. Such a process is used to produce CTL019 T cells for treatment of acute lymphoblastic leukemia (ALL).

The debeading processes and modules disclosed herein may be particularly useful in the manufacture of cells for cell therapy, for example in purifying cells prior to, or after, culture and expansion. For instance, paramagnetic particles coated with anti-CD3 and/or anti CD28 antibodies may be used to selectively expand T cells, for example T cells that are, or will be, modified by introduction of a nucleic acid encoding a chimeric antigen receptor (CAR) or other protein, such that the CAR is expressed by the T cells. During the manufacture of such T cells, the debeading processes or modules may be used to separate T cells from the paramagnetic particles. Such a debeading process or module is used to produce, for example, CTL019 T cells for treatment of acute lymphoblastic leukemia (ALL).

In one such process, illustrated here by way of example, cells, for example, T cells, are collected from a donor (for example, a patient to be treated with an autologous chimeric antigen receptor T cell product) via apheresis (e.g., leukapheresis). Collected cells may then be optionally purified, for example, by an elutriation step, or via positive or negative selection of target cells (e.g., T cells). Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting CD19, is introduced into the cell. The nucleic acid may be introduced in a lentiviral vector. The cells, e.g., the lentivirally transduced cells, may then be expanded for a period of days, for example, 1, 2, 3, 4, 5, 6, 7, 8, 9, 10, or more days, for example in the presence of a suitable medium. After expansion, the debeading processes/modules disclosed herein may be used to separate the desired T cells from the paramagnetic particles. The process may include one or more debeading steps according to the processes of the present disclosure. The debeaded cells may then be formulated for administration to the patient. Examples of CAR T cells and their manufacture are further described, for example, in WO2012/079000, which is incorporated herein by reference in its entirety. The systems and methods of the present disclosure may be used for any cell separation/purification/debeading processes described in or associated with WO2012/079000. Additional CAR T manufacturing processes are described in, e.g., WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of the disclosure are specifically described above, it will be appreciated that modifications and variations of these examples are possible without departing from the spirit and intended scope of the disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate or debead cells to produce a cell product.

Additional manufacturing methods and processes that can be combined with the present invention have been described in the art. For examples, pages 86-91 of WO 2017/117112 describe improved wash steps and improved manufacturing process.

### Sources of Immune Effector Cells

This section provides additional methods or steps for obtaining an input sample comprising desired immune effector cells, isolating and processing desired immune effector cells, e.g., T cells, and removing unwanted materials, e.g., unwanted cells. The additional methods or steps described in this section can be used in combination with any of the elutriation, density gradient centrifugation, selection under flow conditions, or improved wash step described in the preceding sections.

A source of cells, e.g., T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present disclosure, immune effector cells, e.g., T cells, can be obtained from a unit of blood collected from a subject using any number of techniques known to the skilled artisan, and any of the methods disclosed herein, in any combination of steps thereof. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. In another embodiment, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5), Haemonetics Cell Saver Elite (GE Healthcare Sepax or Sefia), or a device utilizing the spinning membrane filtration technology (Fresenius Kabi LOVO), according to the manufacturer's instructions. After washing, the cells may be resuspended in a variety of biocompatible buffers, such as, for example, Ca-free, Mg-free PBS, PlasmaLyte A, PBS-EDTA supplemented with human serum albumin (HSA), or other saline solution with or without buffer. Alternatively, the undesirable components of the apheresis sample may be removed and the cells directly resuspended in culture media.

In one aspect, desired immune effector cells, e.g., T cells, are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, e.g., CD25+ depleted T cells and CD25^{high} depleted T cells, using, e.g., a negative selection technique, e.g., described herein. In some embodiments, the population of T regulatory-depleted cells contains less than 30%, 25%, 20%, 15%, 10%, 5%, 4%, 3%, 2%, 1% of CD25+ cells or CD25^{high} cells.

In one embodiment, T regulatory cells, e.g., CD25+ T cells or CD25^{high} T cells, are removed from the population using an anti-CD25 antibody, or fragment thereof, or a CD25-binding ligand, e.g. IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells or CD25^{high} T cells, are removed from the population using CD25 depleting reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory-depleted cells has 2×10⁹ T regulatory cells, e.g., CD25+ cells or CD25^{high} cells, or less (e.g., 1×10⁹, 5×10⁸, 1×10⁸, 5×10⁷, 1×10⁷, or less T regulatory cells).

In one embodiment, the T regulatory cells, e.g., CD25+ cells or CD25^{high} cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., Treg cells), in a subject prior to apheresis or during manufacturing of a CAR-expressing cell product significantly reduces the risk of subject relapse. For example, methods of depleting Treg cells are known in the art. Methods of decreasing Treg cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) Treg cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete Treg cells prior to manufacturing of the CAR-expressing cell (e.g., T cell, NK cell) product.

Without wishing to be bound by a particular theory, decreasing the level of negative regulators of immune cells (e.g., decreasing the number of unwanted immune cells, e.g., Treg cells), in a subject prior to apheresis or during manufacturing of a CAR-expressing cell product can reduce the risk of a subject's relapse. In an embodiment, a subject is pre-treated with one or more therapies that reduce Treg cells prior to collection of cells for CAR-expressing cell product manufacturing, thereby reducing the risk of subject relapse to CAR-expressing cell treatment. In an embodiment, methods of decreasing Treg cells include, but are not limited to, administration to the subject of one or more of cyclophosphamide, anti-GITR antibody, CD25-depletion, or a combination thereof. In an embodiment, methods of decreasing Treg cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, or a combination thereof, can occur before, during or after an infusion of the CAR-expressing cell product.

In some embodiments, the manufacturing methods comprise reducing the number of (e.g., depleting) Treg cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete Treg cells prior to manufacturing of the CAR-expressing cell (e.g., T cell, NK cell) product.

In an embodiment, a subject is pre-treated with cyclophosphamide prior to collection of cells for CAR-expressing cell product manufacturing, thereby reducing the risk of subject relapse to CAR-expressing cell treatment (e.g., CTL019 treatment). In an embodiment, a subject is pre-treated with an anti-GITR antibody prior to collection of cells for CAR-expressing cell (e.g., T cell or NK cell) product manufacturing, thereby reducing the risk of subject relapse to CAR-expressing cell treatment.

In an embodiment, the CAR-expressing cell (e.g., T cell, NK cell) manufacturing process is modified to deplete Treg cells prior to manufacturing of the CAR-expressing cell (e.g., T cell, NK cell) product (e.g., a CTL019 product). In an embodiment, CD25-depletion is used to deplete Treg cells prior to manufacturing of the CAR-expressing cell (e.g., T cell, NK cell) product (e.g., a CTL019 product).

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory-depleted, e.g., CD25⁺ depleted or CD25^{high} depleted, and tumor antigen depleted cells that are suitable for expression of a CAR, e.g., a CAR described herein. In one embodiment, tumor antigen expressing cells are removed simultaneously with the T regulatory, e.g., CD25+ cells or CD25^{high} cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells or CD25^{high} cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory-depleted, e.g., CD25⁺ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta), e.g., as described herein. In one embodiment, check point inhibitor expressing cells are removed simultaneously with the T regulatory, e.g., CD25+ cells or CD25^{high} cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25⁺ cells or CD25^{high} cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as Dynabeads® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

In one embodiment, a plurality of the immune effector cells of the population do not express diaglycerol kinase (DGK), e.g., is DGK-deficient. In one embodiment, a plurality of the immune effector cells of the population do not express Ikaros, e.g., is Ikaros-deficient. In one embodiment, a plurality of the immune effector cells of the population do not express DGK and Ikaros, e.g., is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one embodiment, the methods of the application can utilize culture media conditions comprising serum-free medium. In one embodiment, the serum free medium is OpTmizer CTS (LifeTech), Immunocult XF (Stemcell technologies), CellGro (CellGenix), TexMacs (Miltenyi), Stemline (Sigma), Xvivo15 (Lonza), PrimeXV (Irvine Scientific), or StemXVivo (RandD systems). The serum-free medium can be supplemented with a serum substitute such as ICSR (immune cell serum replacement) from LifeTech. The level of serum substitute (e.g., ICSR) can be, e.g., up to 5%, e.g., about 1%, 2%, 3%, 4%, or 5%.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Allogeneic CAR-Expressing Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta). Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

Expression systems for siRNA and shRNAs, and exemplary shRNAs, are described, e.g., in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

The CRISPR/Cas system, and uses thereof, are described, e.g., in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

TALENs, and uses thereof, are described, e.g., in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

ZFNs, and uses thereof, are described, e.g., in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386., Aug. 28, 2008), and may be even shorter in manufactured CAR-expressing cells, e.g., CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

Telomerase expression may be stable (e.g., the nucleic acid may integrate into the cell's genome) or transient (e.g., the nucleic acid does not integrate, and expression declines after a period of time, e.g., several days). Stable expression may be accomplished by transfecting or transducing the cell with DNA encoding the telomerase subunit and a selectable marker, and selecting for stable integrants. Alternatively or in combination, stable expression may be accomplished by site-specific recombination, e.g., using the Cre/Lox or FLP/FRT system.

Transient expression may involve transfection or transduction with a nucleic acid, e.g., DNA or RNA such as mRNA. In some embodiments, transient mRNA transfection avoids the genetic instability sometimes associated with stable transfection with TERT. Transient expression of exogenous telomerase activity is described, e.g., in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. Nos. 8,710,200, 8,822,663, 8,680,069, 8,754,062, 8,664,194, or 8680069.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 108)
MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRGCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSDFKTILD

In an embodiment, the hTERT has a sequence at least 80%, 85%, 90%, 95%, 96%, 97%, 98%, or 99% identical to the sequence of SEQ ID NO: 108. In an embodiment, the hTERT has a sequence of SEQ ID NO: 108. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795).

### Activation and Expansion of Immune Effector Cells (e.g., T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of T cells may be expanded by contact with a surface having attached thereto an agent that stimulates a CD3/TCR complex associated signal and an agent that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain aspects, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In one aspect, a ratio of particles to cells of 1:1 or less is used. In one particular aspect, a suitable particle:cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, Dynabeads® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, e.g., a CD19 CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, surface CAR expression, CAR quantitative PCR, or combinations thereof. In one embodiment, the cells, e.g., a CD19 CAR cell described herein, expanded for 5 days show at least a one, two, three or four-fold increase in cells doublings upon antigen stimulation as compared to the same cells expanded in culture for 9 days under the same culture conditions. In one embodiment, the cells, e.g., the cells expressing a CD19 CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD19 CAR cell described herein, expanded for 5 days show at least a one, two, three, four, five, ten-fold or more increase in pg/ml of proinflammatory cytokine production, e.g., IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFNγ, IL-4, IL-7, GM-CSF, IL-10, IL-12, IL-15, TGFβ, and TNFα or any other additives for the growth of cells known to the skilled artisan. Other additives for the growth of cells include, but are not limited to, surfactant, plasmanate, and reducing agents such as N-acetyl-cysteine and 2-mercaptoethanol. Media can include but not limited to RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14-day expansion period, e.g., as measured by a method described herein such as flow cytometry. In one embodiment, the cells are expanded in the presence IL-15 and/or IL-7 (e.g., IL-15 and IL-7). In one embodiment the cells are expanded in the presence of IL-2, IL-15 and IL-21.

In embodiments, methods described herein, e.g., CAR-expressing cell manufacturing methods, comprise removing T regulatory cells, e.g., CD25+ T cells or CD25^{high} T cells, from a cell population, e.g., using an anti-CD25 antibody, or fragment thereof, or a CD25-binding ligand, IL-2. Methods of removing T regulatory cells, e.g., CD25+ T cells or CD25^{high} T cells, from a cell population are described herein. In embodiments, the methods, e.g., manufacturing methods, further comprise contacting a cell population (e.g., a cell population in which T regulatory cells, such as CD25+ T cells or CD25^{high} T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present invention are described in further detail below.

Western blot analysis of CAR expression in primary T cells can be used to detect the presence of monomers and dimers, e.g., as described in paragraph 695 of International Application WO2015/142675, filed Mar. 13, 2015, which is herein incorporated by reference in its entirety.

In vitro expansion of CAR⁺ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8+ T cell subsets by flow cytometry. See, e.g., Milone et al., Molecular Therapy 17(8): 1453-1464 (2009). Alternatively, a mixture of CD4⁺ and CD8+ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28). Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter or a higher version, a Nexcelom Cellometer Vision, Millipore Scepter or other cell counters, following stimulation with αCD3/αCD28 coated magnetic beads on day 0, and transduction with the indicated CAR on day 1.

Animal models can also be used to measure a CAR-expressing cell activity, e.g., as described in paragraph 698 of International Application WO2015/142675, filed Mar. 13, 2015, which is herein incorporated by reference in its entirety.

Dose dependent CAR treatment response can be evaluated, e.g., as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, e.g., as described in paragraph 701 of International Application WO2015/142675, filed Mar. 13, 2015, which is herein incorporated by reference in its entirety. Alternative non-radioactive methods can be utilized as well.

Cytotoxicity can also be assessed by measuring changes in adherent cell's electrical impedance, e.g., using an xCELLigence real time cell analyzer (RTCA). In some embodiments, cytotoxicity is measured at multiple time points.

Imaging technologies can be used to evaluate specific trafficking and proliferation of CARs in tumor-bearing animal models, e.g., as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labelled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on a substrate (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some aspects and embodiments, the compositions and methods herein are optimized for a specific subset of T cells, e.g., as described in US Serial No. PCT/US2015/043219 filed Jul. 31, 2015, the contents of which are incorporated herein by reference in their entireties. In some embodiments, the optimized subsets of T cells display an enhanced persistence compared to a control T cell, e.g., a T cell of a different type (e.g., CD8+ or CD4+) expressing the same construct.

In some embodiments, a CD4+ T cell comprises a CAR described herein, which CAR comprises an intracellular signaling domain suitable for (e.g., optimized for, e.g., leading to enhanced persistence in) a CD4+ T cell, e.g., an ICOS domain. In some embodiments, a CD8+ T cell comprises a CAR described herein, which CAR comprises an intracellular signaling domain suitable for (e.g., optimized for, e.g., leading to enhanced persistence of) a CD8+ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain.

In an aspect, described herein is a method of treating a subject, e.g., a subject having cancer. The method includes administering to said subject, an effective amount of:

1) a CD4+ T cell comprising a CAR (the CARCD4+) comprising:

an antigen binding domain, e.g., an antigen binding domain described herein;

a transmembrane domain; and

an intracellular signaling domain, e.g., a first costimulatory domain, e.g., an ICOS domain; and

2) a CD8+ T cell comprising a CAR (the CARCD8+) comprising:

an antigen binding domain, e.g., an antigen binding domain described herein;

a transmembrane domain; and

an intracellular signaling domain, e.g., a second co stimulatory domain, e.g., a 4-1BB domain, a CD28 domain, or another costimulatory domain other than an ICOS domain;

wherein the CARCD4+ and the CARCD8+ differ from one another.

Optionally, the method further includes administering:

3) a second CD8+ T cell comprising a CAR (the second CARCD8+) comprising:

an antigen binding domain, e.g., an antigen binding domain described herein;

a transmembrane domain; and

an intracellular signaling domain, wherein the second CARCD8+ comprises an intracellular signaling domain, e.g., a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### Pharmaceutical Compositions and Treatments

In some aspects, the disclosure provides a method of treating a patient, comprising administering CAR-expressing cells produced as described herein, optionally in combination with one or more other therapies. In some aspects, the disclosure provides a method of treating a patient, comprising administering a reaction mixture comprising CAR-expressing cells as described herein, optionally in combination with one or more other therapies. In some aspects, the disclosure provides a method of shipping or receiving a reaction mixture comprising CAR-expressing cells as described herein. In some aspects, the disclosure provides a method of treating a patient, comprising receiving a CAR-expressing cell that was produced as described herein, and further comprising administering the CAR-expressing cell to the patient, optionally in combination with one or more other therapies. In some aspects, the disclosure provides a method of treating a patient, comprising producing a CAR-expressing cell as described herein, and further comprising administering the CAR-expressing cell to the patient, optionally in combination with one or more other therapies. The other therapy may be, e.g., a cancer therapy such as chemotherapy.

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In one embodiment, a therapy described herein, e.g., a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In one embodiment, the GITR binding molecules and/or molecules modulating GITR functions (e.g., GITR agonist and/or Treg depleting GITR antibodies) are administered prior to the CAR-expressing cell. For example, in one embodiment, a GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as ALL or CLL). In one embodiment, the subject has CLL. In embodiments, the subject has ALL. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells. In one embodiment, the subject has CLL.

The methods described herein can further include formulating a CAR-expressing cell in a pharmaceutical composition. Pharmaceutical compositions may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated, e.g., for intravenous administration.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In some embodiments, a dose of CAR cells (e.g., CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises about 1.1×10⁶-1.8×10⁷ cells/kg. In some embodiments, a dose of CAR cells (e.g., CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In certain aspects, it may be desired to administer activated immune effector cells (e.g., T cells, NK cells) to a subject and then subsequently redraw blood (or have an apheresis performed), activate immune effector cells (e.g., T cells, NK cells) therefrom, and reinfuse the patient with these activated and expanded immune effector cells (e.g., T cells, NK cells). This process can be carried out multiple times every few weeks. In certain aspects, immune effector cells (e.g., T cells, NK cells) can be activated from blood draws of from 10 cc to 400 cc. In certain aspects, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, e.g., by intradermal or subcutaneous injection. The compositions of immune effector cells (e.g., T cells, NK cells) may be injected directly into a tumor, lymph node, or site of infection.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Improved CAR T Manufacturing Process

### Methods to Improve CAR T Cell Manufacturing Process

### Introduction and Short Description of Invention

This example describes novel methods to expand T cells to improve the outcome of CAR T cell manufacturing for T cells with low naïve, high effector, high senescent starting T cell populations.

A critical phenotypic and genetic change as T cells become late-differentiated and progress to senescence is the loss of expression of CD28, an essential co-stimulatory receptor that activates T cell pathways such as the Akt and NFκB pathways, and modulates important functions such as lipid raft formation, IL-2 gene transcription, apoptosis, stabilization of cytokine mRNA, glucose metabolism and cell adhesion. Therefore, downregulation of CD28 would fundamentally alter normal T cell function, survival, and proliferation (Thompson, C., et al., Proc. Natl. Acad. Sci. USA, Vol. 86, pp. 1333-1337, 1989; Azuma, M., et al., J. Immunol., Vol. 150, pp. 1147-1159, 1993; Weng, N., et al., Trends in Immunology, Vol. 130, pp. 306-312, 2009, herein incorporated by reference in their entireties).

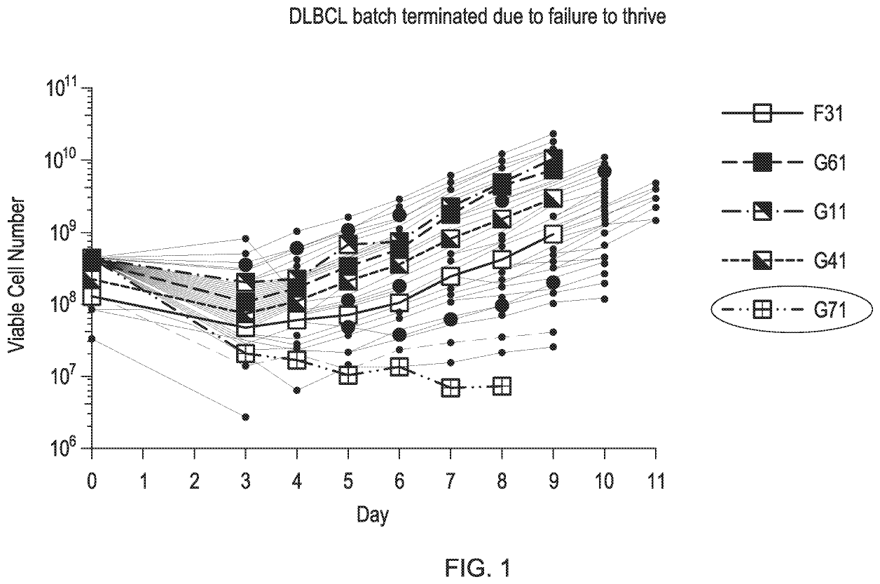

Clinical and commercial CAR T manufacturing experience confirmed that batches characterized by low naïve, high effector and senescent starting T cell populations were more likely to fail due to low cell growth. FIG. 1 and Table 3 show one such example. A T cell batch isolated from a diffuse large B-cell lymphoma (DLBCL) patient (designated as sample “G71”) showed low naïve T cells, high effector T cells and effector memory T cells, as well as low CD28+ T cells (Table 3). This T cell batch failed to expand using the Bead CAR T manufacturing process described in this Example below (FIG. 1).

TABLE 3
T cell phenotypes of sample G71 at Day 0

| | CD4 | CD8 |
|---|---|---|
| Naïve | 1.1% | 1.7% |
| Effector + Effector memory | 89.4% | 95.9% |
| CD28+ | 42.2% | 8.8% |

Therefore, in order to improve the manufacturing outcome for low naïve and high effector/senescent starting T cells, the following procedures are recommended:

1. Bead-free stimulation using CD3/CD28 conjugated colloidal matrix (TransAct, Miltenyi Biotec or similar reagents);

2. Supplement growth media with a combination of IL-2, IL-15, and IL-21.

Implementation of this new manufacturing process (bead-free CD3/CD28 activation, plus culturing cells with IL-2, IL-15, and IL-21; also referred to as the “bead-free stimulation and cytokine (BFSC) process”) has been shown to achieve higher target cell yields with less features of senescence and exhaustion in the CAR T cell product compared to the Bead CAR T manufacturing process described in this Example below.

### Bead CAR T Cell Manufacturing Process

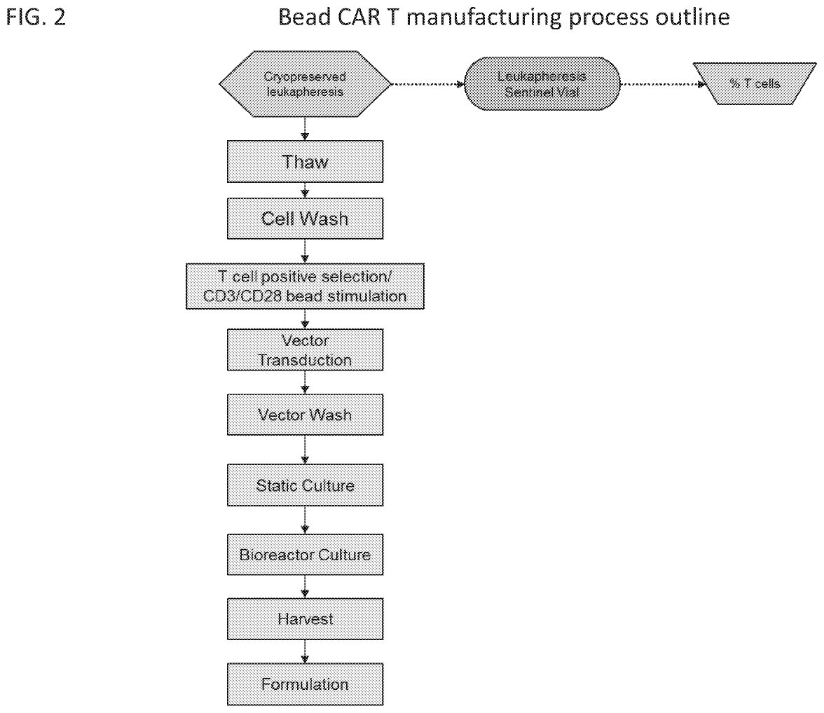

The so-called Bead CAR T cell manufacturing process is outlined in FIG. 2 and described below. In some embodiments, the Bead CAR T cell manufacturing process includes the following steps:

(a) providing an apheresis, e.g., leukapheresis, product,

(b) isolating a population of cells (e.g., T cells) from the apheresis product and contacting the population of cells (e.g., T cells) using anti-CD3 and anti-CD28 antibodies coupled to Dynabeads,

(c) contacting the population of cells (e.g., T cells) with IL-2,

(d) contacting the population of cells (e.g., T cells) with a nucleic acid molecule (e.g., a DNA or RNA molecule) encoding the CAR, thereby providing a population of cells (e.g., T cells) comprising the nucleic acid molecule, and

(e) expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells. Each of these steps is described in more detail below.

The manufacturing of CAR T cell product starts with cryopreserved apheresis product. Initial samples of fresh apheresis products are taken before freezing at the collection site for testing, including cell counting, determination of percent of viable cells, and immunophenotyping (FIG. 2).

The manufacturing process starts on Day 0 with thawing of the cryopreserved leukapheresis. The thawed leukapheresis is then washed to remove cryopreservation medium. The cell suspension then proceeds directly to T cell selection/stimulation. The stimulation of T cells is performed using immunomagnetic beads bearing anti-CD3/CD28 monoclonal antibodies, Dynabeads® CD3/CD28 CTS™. The cell-bead suspension then undergoes magnetic separation, retaining the bead-bound CD3+/CD45+ T-cell fraction. The bead-bound cells in this positive fraction are advanced to lentiviral transduction. Lentiviral transduction is performed twice (FIG. 2).

Following lentiviral transduction, the cell culture is washed to remove non-integrated vector and residual viral particles. The washed cells are seeded into culture bags, placed into static culture and maintained in culture at a defined target concentration. When the cell count reaches the required minimum total viable cells, the cell culture is seeded into the bioreactor cell bag, in which ex vivo cell expansion continues. To support high cell concentration, perfusion culture is initiated when the target viable cell count is reached at a culture volume of 1 L. During perfusion, the medium is changed daily dependent on the cell density of the culture, with cell-free medium removed via the filter in the bioreactor and fresh medium introduced. From the start of the process through the harvest, CAR T cells, e.g., CTL019 cells, are expanded ex vivo for at least 3, 4, 5, 6, 7, 8, 9, or 10 days and harvested. On the harvest day, the Dynabeads® CD3/CD28 CTS™ are first separated from the cell suspension using a magnetic separation device, and the cell suspension is washed to further remove residual vector and viral particles, culture media and cell debris. The cell suspension is then centrifuged, the supernatant removed, and the cell pellet re-suspended in the formulation medium (FIG. 2). The doses are then distributed into individual cryobags and frozen via controlled rate freezer. Cryopreserved CAR T, e.g., CTL019, bags are then stored in vapor-phase of liquid nitrogen in monitored LN2 storage tanks, in a secure, limited access area until final release and shipping.

Additional embodiments for the Bead CAR T cell manufacturing process are disclosed in WO201207900, WO2016109410, WO2017/117112, McGuirk J, et al., Cytotherapy. 2017; 19(9):1015-24, and Buechner J, et al., HemaSphere. 2018; 2(1):e18, incorporated herein by reference in their entireties.

### Experimental Data, Process Development and Final Product Qualities

Senescent and exhausted T cells have been identified as a T cellular subtype that negatively affects CART manufacturing. Senescent and exhausted T cells undergo suboptimal activation and growth potentially due to cellular immune deficiencies. The current manufacturing experience has demonstrated that the presence of senescent and exhausted T cells in the patient leukapheresis material reduce the population doubling level (PDL).

The Bead CAR T cell manufacturing process was evaluated for feasibility using chronic lymphoblastic leukemia (CLL) patient material as this material was shown to demonstrate T cell defects that were likely to impact T cell proliferation and the manufacturing success outcome (Riches, J., et al., Seminars in Cancer Biology, Vol. 20, pp. 431-438, 2010; Fraietta, J., et al., Nature Medicine, Vol. 24, pp. 563-5′71, 2018, herein incorporated by reference in their entireties). Data was originally obtained using material from three different CLL patients (all small scale). In the BFSC process described in this Example, several changes were made to the original process and were shown to improve the expansion rate in small scale. These changes include:
Performing T cell negative selection in the place of CD3/CD28 DynaBeads positive selection;
Contacting T cells using bead-free CD3/CD28 T cell activator (TransAct); and
Supplementing modified media (MM) originally containing IL-2 with additional cytokines IL-15 and IL-21.

The new process was then scaled up, and successful expansion was then performed using healthy donor material and material from another CLL patient.

In this work, IL-15 and IL-21 were used to supplement IL-2 in the modified media (MM) and WAVE modified media (WMM). MM supplemented with IL-15 and IL-21 was tested using CLL patient materials and was shown to be superior in comparison with MM-only for cell growth (FIG. 3), paired t-test p-value=0.007.

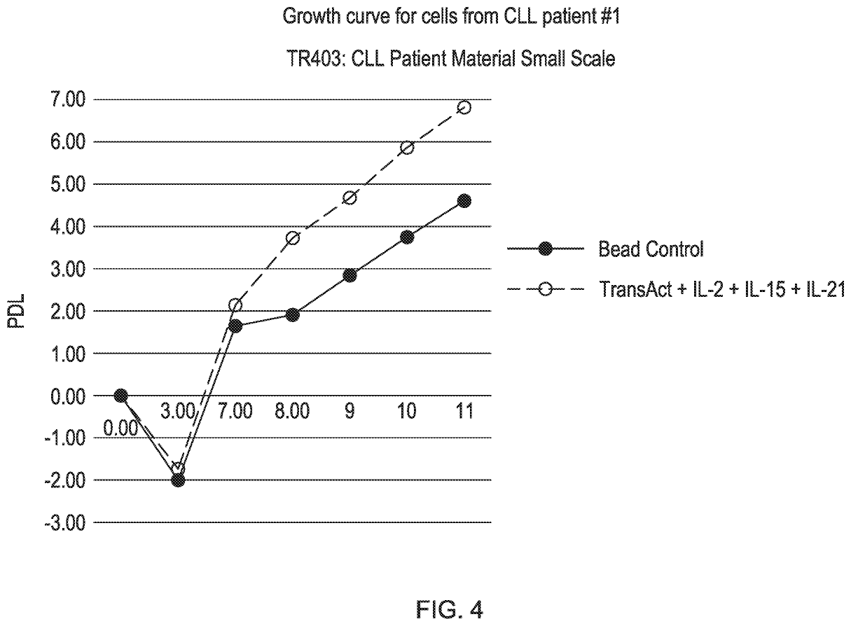

Material from three CLL patients was used to compare this BFSC process (bead-free CD3/CD28 activation using TransAct, plus culturing cells with IL-2, IL-15, and IL-21; referred to as “TransAct+IL-2+IL-15+IL-21” in FIGS. 4-9) with a bead-free control process (bead-free CD3/CD28 activation using TransAct, plus culturing cells with IL-2 but not IL-15 or IL-21; referred to as “TransAct” in FIGS. 8-9) or a bead control process (referred to as “Bead control” in FIGS. 4-9). In the so-called “bead control” process, cells were stimulated using immunomagnetic beads bearing anti-CD3/CD28 monoclonal antibodies, Dynabeads® CD3/CD28 CTS™, and were cultured with IL-2 but not IL-15 or IL-21. Cell samples from all three CLL patients had T cell percentages lower than normal donors: the highest was 24.0%, and the lowest was 1.5% (Tables 4, 6, and 8). All three CLL patients had starting T cell phenotypes different from healthy donors: low CD4/CD8 ratios, and high effector and senescent T cell percentages (Tables 5, 7, and 9). The BFSC process has demonstrated higher T cell proliferation levels in comparison with the bead control process in cell samples from all three CLL patients (FIGS. 4-6).

TABLE 4
Cell percentages of sample TR403 at Day 0

| % T cells | % B cells | % NK cells | % Monocytes |
|---|---|---|---|
| 3.52% | 95.5% | 0.90% | 0.02% |

TABLE 5
T cell phenotypes of sample TR403 at Day 0

| T cell phenotypes | CD4 | CD8 |
|---|---|---|
| Naïve/Tscm | 34.7% | 25.7% |
| E + EM (terminally maturated) | 32.4% | 70.1% |
| Senescent (CD28−/CD27−/CD57+) | 0.4% | 34.1% |
| Exhausted (PD-1+) | 11.4% | 34.1% |
| CD28+ | 97.4% | 68.0% |

| | |
|---|---|
| CD4/CD8 ratio | 0.72 |

TABLE 6
Cell percentages of sample TR411 at Day 0

| % T cells | % B cells | % NK cells | % Monocytes |
|---|---|---|---|
| 24.0% | 58.52% | 1.65% | 0.04% |

TABLE 7
T cell phenotypes of sample TR411 at Day 0

| T cell phenotypes | CD4 | CD8 |
|---|---|---|
| Naïve/Tscm | 7.8% | 6.2% |
| E + EM (terminally maturated) | 78.2% | 91.0% |
| Senescent (CD28−/CD27−/CD57+) | 18.7% | 30.1% |
| Exhausted (PD-1+) | 14.4% | 9.5% |
| CD28+ | 70.5% | 34.3% |

| | |
|---|---|
| CD4/CD8 ratio | 0.39 |

TABLE 8
Cell percentages of sample TR412 at Day 0

| % T cells | % B cells | % NK cells | % Monocytes |
|---|---|---|---|
| 1.5% | 98.2% | 0.14% | 0.02% |

TABLE 9
T cell phenotypes of sample TR412 at Day 0

| T cell phenotypes | CD4 | CD8 |
|---|---|---|
| Naïve/Tscm | 49.2% | 10.2% |
| E + EM (terminally maturated) | 25.6% | 87.6% |
| Senescent (CD28−/CD27−/CD57+) | 14.1% | 39.0% |
| Exhausted (PD-1+) | 37.4% | 57.9% |
| CD28+ | 98.1% | 31.1% |

| | |
|---|---|
| CD4/CD8 ratio | 0.66 |

CD28 is gradually lost with age or as a result of repetitive T cell activations. CD8+ T cells experience a more dramatic decrease in CD28 expression compared to CD4+ T cells. CD28 decrease was observed to different levels in all three CLL starting materials. However, with the BFSC process, and not with the bead control process, CD28 level in the final CAR T product was restored (FIG. 7). Therefore, final products from the BFSC process had less senescent phenotypes than the products from the bead control process.

Inhibitory receptors on immune cells are pivotal regulators of immune escape in cancer. PD-1 and LAG-3, two inhibitory receptors, that are commonly expressed on exhausted T cells, can act synergistically in inhibiting T cell response to tumors (Woo, S-R., et al., Cancer Research, Vol. 72, pp. 917-927, 2012, herein incorporated by reference in its entirety). Comparison of the final products originating from the BFSC process, bead-free control process, and bead control process reveals that both PD-1 expression and double PD-1/LAG-3 expression were significantly lower in CAR T cells manufactured using the BFSC process (FIGS. 8 and 9). According to Fraietta, J., et al., Nature Medicine, Vol. 24, pp. 563-571, 2018, analysis of PD-1 co-expression with LAG-3 revealed that CD8+LAG-3+ CTL019 cells co-expressing PD-1 were associated with poor responses, whereas individuals who had complete and durable remissions were infused with products containing significantly lower frequencies of these cells. Therefore, the product originating from the BFSC process had less exhausted and potentially more functional phenotype than the product originating from the bead control process.

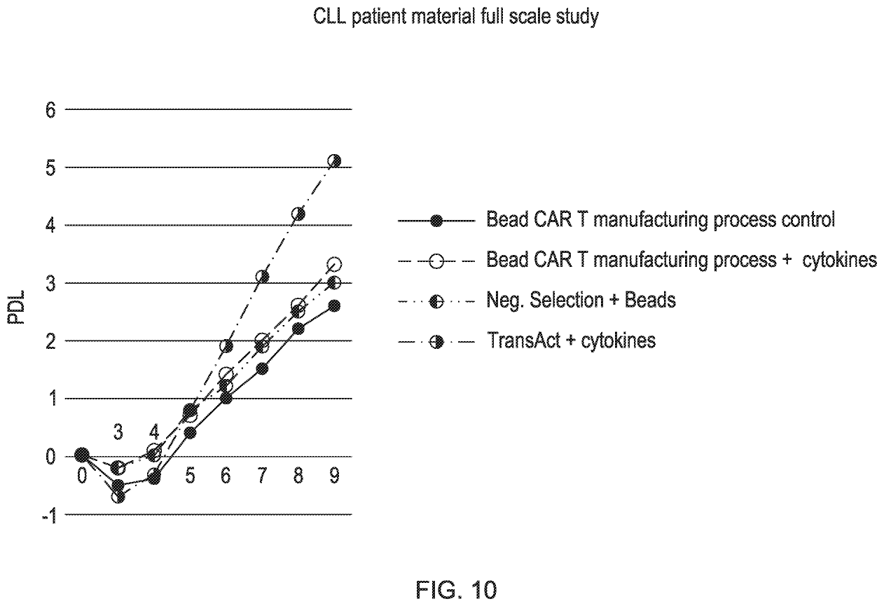

The BFSC process was scaled up for expansion of another CLL patient material full scale. The starting CLL material was characterized by low naïve T cells, high effector and exhausted T cells, low CD4/CD8 ratio, and low CD28 expression (Tables 10 and 11). Four different manufacturing processes were compared: the Bead CAR T cell manufacturing process (referred to as “Bead CAR T cell manufacturing control” in FIG. 10), a modified Bead CAR T cell manufacturing process in which the cell culture medium, which contained IL-2 originally, was further supplemented with IL-15 and IL-21 (referred to as “Bead CAR T cell manufacturing process+cytokines” in FIG. 10), a third manufacturing process in which T cells were negatively selected, stimulated with CD3/CD28 Dynabeads, and cultured with IL-2 but not IL-15 or IL-21 (referred to as “Neg. Selection+Beads” in FIG. 10), and the BFSC process (referred to as “TransAct+cytokines” in FIG. 10). T cells from this CLL patient showed much stronger expansion under the BFSC process than any of the remaining three processes (FIG. 10).

TABLE 10
Cell percentages at Day 0

| % T cells | % B cells | % NK cells | % Monocytes |
|---|---|---|---|
| 6.85% | 89.9% | 1.61% | 1.20% |

TABLE 11
T cell phenotypes at Day 0

| T cell phenotypes | CD4 | CD8 |
|---|---|---|
| Naïve/Tscm | 8.83% | 4.10% |
| E + EM (terminally maturated) | 52.8% | 89.3% |
| Senescent (CD28−/CD27−/CD57+) | 18.2% | 7.87% |
| Exhausted (PD-1+) | 52.2% | 37.3% |
| CD28+ | 58.5% | 44.1% |

| | |
|---|---|
| CD4/CD8 ratio | 0.63 |

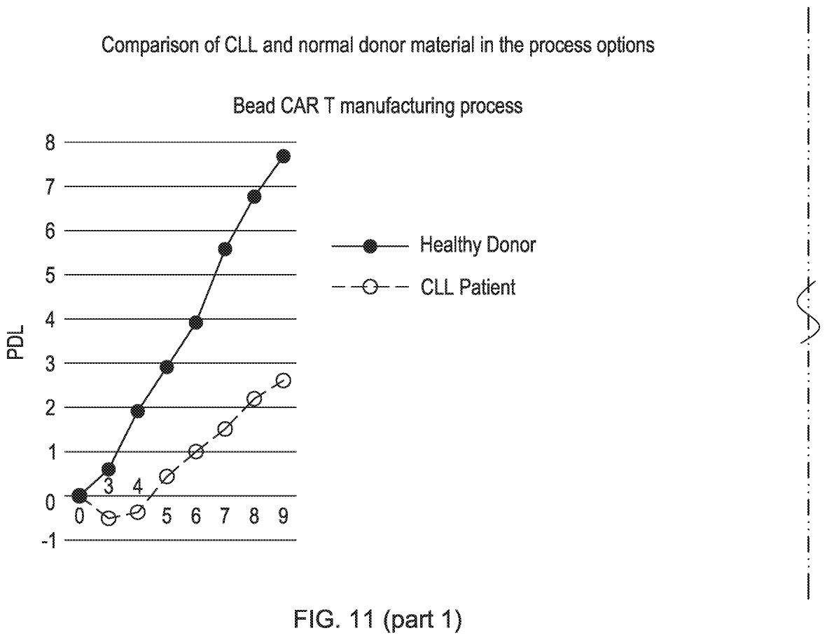

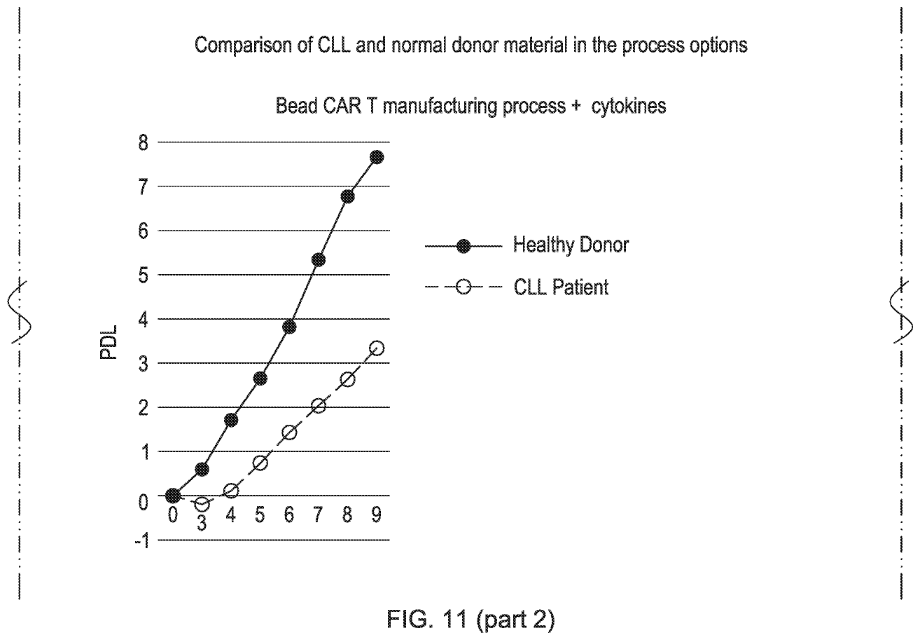

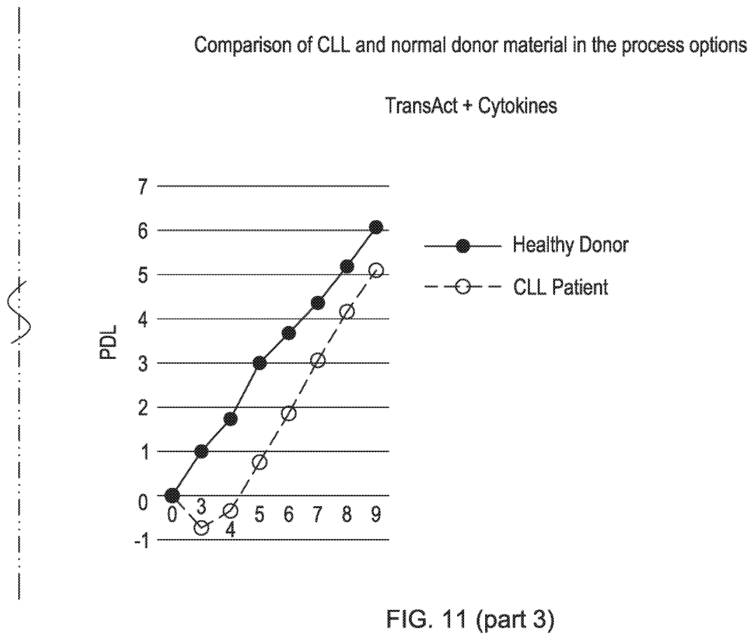

The BFSC process was most efficient for expansion of the CLL patient T cells. Notably, when the Bead CAR T cell manufacturing process (FIG. 11, the left panel) or the modified Bead CAR T cell manufacturing process (FIG. 11, the middle panel) was used, T cells from a CLL patient showed much less proliferation than T cells from a healthy donor. In contrast, under the BFSC process, the expansion level of T cells from the CLL patient was comparable with that of healthy donor T cells (FIG. 11, the right panel).

### Process of the Bead-Free Stimulation and Cytokine (BFSC) Process

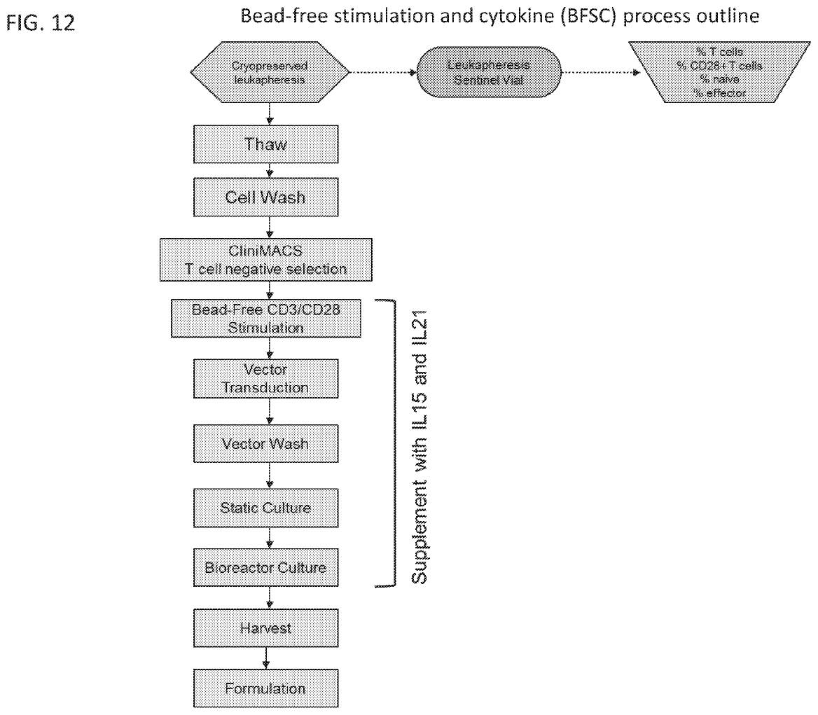

Based on the studies described above, an updated CAR T manufacturing process for, e.g., CTL019, has been laid out in FIG. 12.

The manufacturing of CAR T cell product starts with cryopreserved apheresis product. Initial samples of fresh apheresis products are taken before freezing at the collection site for testing, including cell counting, determination of percent of viable cells, and immunophenotyping including T cell subtypes and CD28 expression. The manufacturing process starts with thawing of the cryopreserved leukapheresis. The thawed leukapheresis is then washed to remove cryopreservation medium followed by addition of magnetic beads for T cell negative selection. After the incubation excessive beads are removed through a wash procedure. The cell suspension then proceeds to T cell selection on CliniMACS using negative selection protocol. Negative fraction is collected and washed again to remove the CliniMACS buffer and cells are re-suspended in MM, which contains IL-2, and placed in a static culture bag. Bead-free CD3/CD28 activator (TransAct) is added following the manufacturer's recommendation. The culture is additionally supplemented with IL-15 and IL-21 and incubated at 37° C. and 5% CO₂ for 24 hours before it is advanced to lentiviral transduction. Lentiviral transduction is performed once, on Day 1.

Following lentiviral transduction, the cell culture is washed to remove non-integrated vector and residual viral particles. The washed cells are seeded into culture bags, placed into static culture and maintained in culture at a defined target concentration. The culture is continued until the cell number is sufficient to enable seeding into the bioreactor. When the cell count reaches the required minimum total viable cells, the cell culture is seeded into the bioreactor cell bag, in which ex vivo cell expansion continues. To support high cell concentration, perfusion culture is initiated when the target viable cell count is reached at a culture volume of 1 L. During perfusion, the medium is changed daily dependent on the cell density of the culture, with cell-free medium removed via the filter in the bioreactor and fresh medium introduced. The cultures are supplemented with IL-15 and IL-21 following the schedule in Table 12 for the duration of the culture. From the start of the process through the harvest, CAR T cells, e.g., CTL019 cells, are expanded ex vivo for at least 3, 4, 5, 6, 7, 8, 9, or 10 days and harvested. On the harvest day, the cell suspension is washed to further remove residual vector and viral particles, culture media and cell debris, after which the cell suspension proceeds to formulation. The doses are then distributed into individual cryobags and frozen via controlled rate freezer. Cryopreserved CAR T, e.g., CTL019, bags are then stored in vapor-phase of liquid nitrogen in monitored LN2 storage tanks.

TABLE 12
Activation schedule

| | Day 0 | Day 3 | After Day3 |
|---|---|---|---|
| TransAct | Use vendor | None | None |
| | recommendation | | |
| IL-2 | Part of MM | Part of MM | Part of MM, |
| | formulation | formulation | MM200, WMM500 |
| | | | formulations |
| IL-15 | added to media | added to media | added to media |
| IL-21 | added to media | added to media | added to media |

### Identification of the Attributes of the Starting Apheresis Material Indicative of Bead CAR T Cell Manufacturing Process Replacement by the BFSC Process

Utilization of the BFSC process is most beneficial and provides clear difference for the incoming material meeting the following three conditions:
Low (single digit percentage) naïve CD4+ and CD8+ T cell content;
High T cell effector content (above 50% for both CD4+ and CD8+ T cells);
Low CD28 expression (below 50% for both CD4+ and CD8+ T cells).

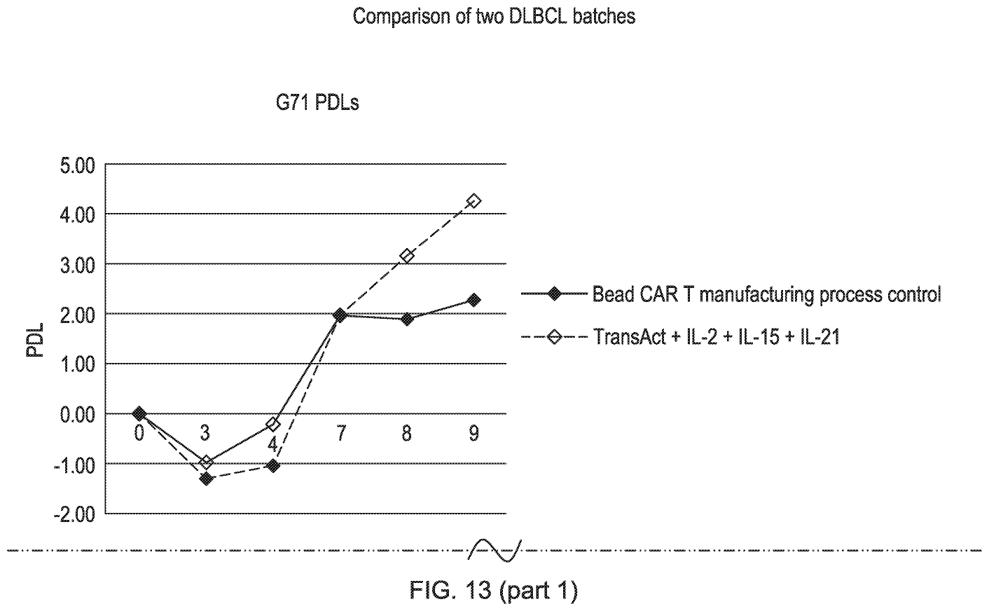

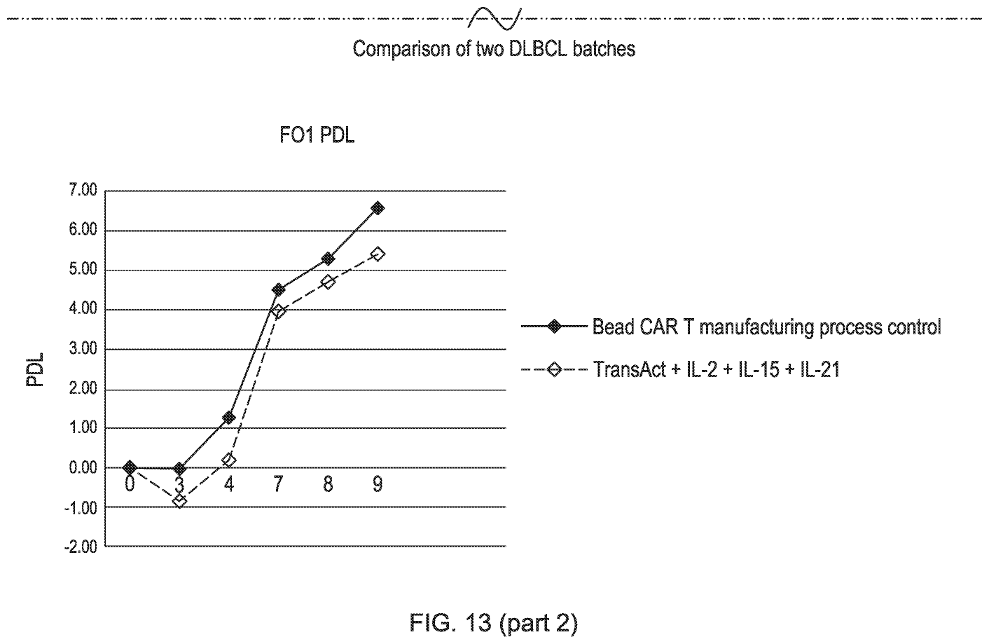

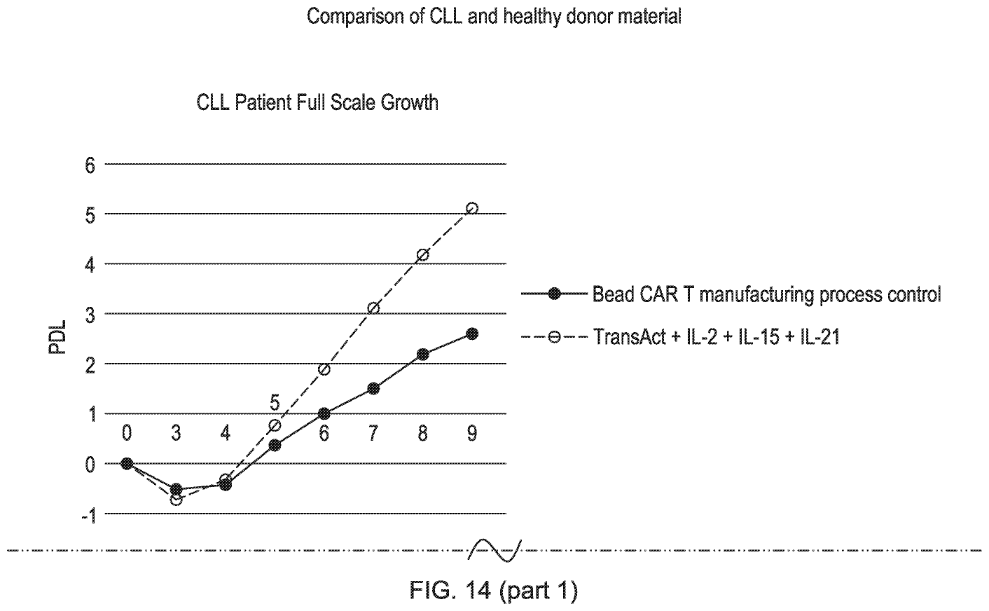

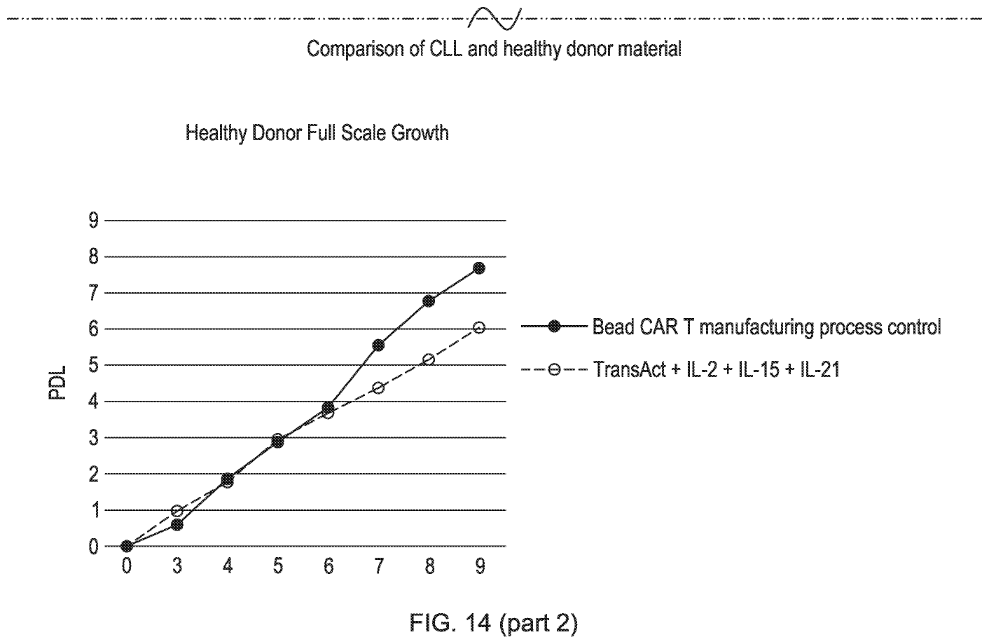

If T cells have more “normal” phenotypes, then both the Bead CAR T cell manufacturing process (referred to as “Bead CAR T cell manufacturing process control” in FIGS. 13 and 14) and the BFSC process (referred to as “TransAct+IL-2+IL-15+IL-21” in FIGS. 13 and 14) can render similar T cell proliferation levels.

For example, as shown in Table 13, sample G71 meets the three conditions described above, and it showed much stronger T cell proliferation under the BFSC process than the Bead CAR T cell manufacturing process (FIG. 13, upper panel). In contrast, sample F01, which has more “normal” T cell phenotypes as shown in Table 13, showed similar T cell proliferation under both approaches (FIG. 13, lower panel). A similar phenomenon was observed when comparing T cells from a CLL patient and T cells from a healthy donor (Table 14 and FIG. 14).

TABLE 13
T cell phenotypes of samples
G71 and F01 at Day 0

| | |
|---|---|
| CD4 | CD8 |

G71

| | | |
|---|---|---|
| Naïve | 1.1% | 1.7% |
| Effector + Effector memory | 89.4% | 95.9% |
| CD28+ | 42.2% | 8.8% |

F01

| | | |
|---|---|---|
| Naïve | 14.1% | 16.4% |
| Effector + Effector memory | 20.6% | 55.4% |
| CD28+ | 99.1% | 64.1% |

TABLE 14
T cell phenotypes of CLL and
healthy donor material at Day 0

| | |
|---|---|
| CD4 | CD8 |

CLL material

| | | |
|---|---|---|
| Naïve | 8.83% | 4.10% |
| Effector + Effector memory | 52.8% | 89.3% |
| CD28+ | 58.5% | 44.1% |

Healthy donor material

| | | |
|---|---|---|
| Naïve | 34.1% | 32.9% |
| Effector + Effector memory | 31.7% | 64.8% |
| CD28+ | 88.4% | 44.9% |

### Example 2: Characterization of Tisagenlecleucel, a CAR T Cell Product Manufactured from Patients with Pediatric ALL

Tisagenlecleucel comprises T cells expressing a CAR that recognizes CD19, a protein found exclusively on B cells. 51 batches of tisagenlecleucel that were manufactured for the ELIANA, a phase 2 trial of tisagenlecleucel in pediatric and young adult patients with r/r ALL, were characterized. Tisagenlecleucel characteristics were evaluated by flow cytometry for identification of cell phenotypes and T-cell subsets and for their functional response to CD19 expressing target cells by means of cytokine release (ELISA) and cytotoxicity. Tisagenlecleucel contained mostly T cells despite very highly variable cell composition of the leukapheresis material. No B cells/B lymphoblasts/other cells originating from the leukapheresis starting material and only occasional residual NK cells were detected. The relative amount of CAR-positive cells was covering a wide range resulting in varying mixes of non-transduced (CAR-negative) and CAR-positive CD4+ and CD8+ cells. The ratio of transduced CAR-positive CD4+:CD8+ cells was also varying and no relation to clinical outcome in pediatric ALL was observed. The T cells in tisagenlecleucel were predominantly less mature transduced T cells which were highly activated. Immunophenotyping showed high cellular fitness with minimal immunosenescent or exhausted phenotype. Tisagenlecleucel demonstrated functional activity upon CD19-specific stimulation resulting in IFN-gamma release and cytotoxic capacity, however with a wide range among different batches. Despite the vast heterogeneity in regard to key product attributes such as T cell composition and functional response, tisagenlecleucel resulted in high rates of tumor remissions with 83% CR/CRi.

### EQUIVALENTS

The disclosures of each and every patent, patent application, and publication cited herein are hereby incorporated herein by reference in their entireties. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of making a population of cells (e.g., T cells) that express a chimeric antigen receptor (CAR), the method comprising:
(i) contacting a population of cells (e.g., T cells) with IL-2 and one or both of: IL-15 and IL-21, and
(ii) contacting the population of cells (e.g., T cells) with a nucleic acid molecule (e.g., a DNA or RNA molecule) encoding the CAR, thereby providing a population of cells (e.g., T cells) comprising the nucleic acid molecule, wherein:
the population of cells at the beginning of step (i) has one or more (e.g., 2, 3, 4, 5, 6, 7, 8, 9, 10, 11, 12, 13, 14, or all) of the following properties:
(1) the population of cells at the beginning of step (i) does not expand or expands for no more than 5, 6, 7, 8, or 9-fold over 8-11 days using the Bead CART cell manufacturing process described in Example 1,
(2) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than 5, 6, 7, 8, 9, 10, 15, 20, 25, 30, 40, 50, or 60%, e.g., lower than 10%,
(3) the percentage of naïve T cells and/or Tscm among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, or 90% lower than the corresponding value in a reference population of cells,
(4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than 20, 25, 30, 40, 50, 60, 70, 80, 90, or 95%, e.g., higher than 50%,
(5) the percentage of Teff cells and/or Tem cells among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 600, 700, 800, or 900% higher than the corresponding value in a reference population of cells,
(6) the percentage of CD28+CD4+ T cells among CD4+ T cells in the population of cells at the beginning of step (i) is no more than 40, 45, 50, 55, 60, 65, 70, 75, 80, 85, 90, or 95%, e.g., no more than 50%,
(7) the percentage of CD28+CD8+ T cells among CD8+ T cells in the population of cells at the beginning of step (i) is no more than 30, 35, 40, 45, 50, 55, 60, 65, or 70%, e.g., no more than 50%,
(8) the percentage of CD28+ T cells among T cells, the percentage of CD28+CD4+ T cells among CD4+ T cells, or the percentage of CD28+CD8+ T cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CART cell manufacturing process described in Example 1), e.g., at least 20, 30, 40, 50, 60, 70, 80, or 90% lower than the corresponding value in a reference population of cells,
(9) the ratio of CD4+ T cells to CD8+ T cells in the population of cells at the beginning of step (i) is lower than 0.5, 0.8, 1, 1.2, or 1.5,
(10) the ratio of CD4+ T cells to CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, or 99% lower than the corresponding value in a reference population of cells,
(11) the percentage of CD4+ senescent cells (e.g., CD28−CD27−CD57+CD4+ senescent cells) among CD4+ T cells in the population of cells at the beginning of step (i) is more than 2, 5, 10, 15, 20, 30, 40, 50, 60, 70, 80, 90 or 95%,
(12) the percentage of CD8+ senescent cells (e.g., CD28−CD27−CD57+CD8+ senescent cells) among CD8+ T cells in the population of cells at the beginning of step (i) is more than 20, 25, 30, 35, 40, 50, 60, 70, 80, 90 or 95%,
(13) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000, or 9500% higher than the corresponding value in a reference population of cells,
(14) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than 10, 20, 30, 40, 50, 60, 70, 80 or 90%, and
(15) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000 or 9000% higher than the corresponding value in a reference population of cells.

2. The method of claim 1, wherein step (i) comprises contacting the population of cells (e.g., T cells) with IL-2, IL-15, and IL-21.

3. The method of claim 1 or 2, wherein step (ii) is performed after step (i), e.g., about 0.5, 1, 1.5, or 2 days after the beginning of step (i), e.g., about 1 day after the beginning of step (i).

4. The method of any one of claims 1-3, further comprising:
(iii) contacting (e.g., binding) the population of cells (e.g., T cells) with an agent that stimulates a CD3/TCR complex and/or an agent that stimulates a costimulatory molecule, optionally wherein:
the agent that stimulates a CD3/TCR complex is an agent that stimulates CD3 (e.g., the agent that stimulates a CD3/TCR complex comprises an anti-CD3 antibody), and
the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof (e.g., the agent that stimulates a costimulatory molecule comprises an anti-CD28 antibody).

5. The method of claim 4, wherein the agent that stimulates a CD3/TCR complex or the agent that stimulates a costimulatory molecule does not comprise a bead.

6. The method of claim 4 or 5, wherein: the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix.

7. The method of any one of claims 4-6, wherein step (iii) comprises contacting (e.g., binding) the population of cells (e.g., T cells) with T Cell TransAct™.

8. The method of any one of claims 4-7, wherein step (iii) is performed together with step (i), or no more than 1, 2, 3, 4, 5, or 6 hours prior to or after step (i).

9. The method of any one of claims 1-8, wherein the population of cells at the beginning of step (i) is isolated from apheresis material, e.g., by reducing monocytes (e.g., CD14+ cells), B cells (e.g., CD19+ cells), and/or NK cells (e.g., CD56+ cells) from the apheresis material, e.g., using CliniMACS, optionally wherein the apheresis material is leukapheresis material (e.g., fresh or frozen leukapheresis material).

10. The method of claim 9, wherein the apheresis, e.g., leukapheresis, material is isolated from a subject having cancer.

11. The method of claim 9 or 10, wherein the apheresis, e.g., leukapheresis, material is isolated from a subject, cryopreserved after being isolated from the subject, and thawed before the population of cells at the beginning of step (i) is isolated from the apheresis, e.g., leukapheresis material.

12. The method of any one of claims 9-11, wherein the apheresis, e.g., leukapheresis, material has one or both of the following properties:
(a) the percentage of T cells in the apheresis, e.g., leukapheresis, material is no more than 1, 5, 10, 15, 20, 25, 30, 35, or 40%, and
(b) the percentage of T cells in the apheresis, e.g., leukapheresis, material is lower than the corresponding value in reference apheresis, e.g., leukapheresis, material (e.g., apheresis, e.g., leukapheresis, material from a healthy donor), e.g., at least 20, 30, 40, 50, 60, 70, 80, or 90% lower than the corresponding value in reference apheresis, e.g., leukapheresis, material.

13. The method of any one of claims 1-12, further comprising after step (ii):
(iv) expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells.

14. The method of claim 13, wherein step (i) comprises contacting the population of cells (e.g., T cells) with IL-2, IL-15, and IL-21, and wherein the expansion of the population of expanded cells at the end of step (iv) relative to the population of cells at the beginning of step (i) is greater (e.g., at least 5, 10, 15, 20, 25, 30, 40, 50, 100, 200, 300, 400 or 500% greater) than the expansion of a population of cells made by an otherwise similar method in which the population of cells is not contacted with IL-2, IL-15, or IL-21, or is contacted with IL-2 but not IL-15 or IL-21, e.g., as measured by population doubling level (PDL), e.g., as assessed using methods described in Example 1 with respect to FIGS. 3-6, and 10.

15. The method of claim 13 or 14, wherein step (i) comprises contacting the population of cells (e.g., T cells) with IL-2, IL-15, and IL-21, and wherein the percentage of CD28+ T cells among T cells (e.g., the percentage of CD28+CD8+ T cells among CD8+ T cells, or the percentage of CD28+CD4+ T cells among CD4+ T cells) in the population of expanded cells at the end of step (iv) is higher (e.g., at least 5, 10, 15, 20, 25, 30, 40, 50, 60, 70, or 80% higher) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIG. 7.

16. The method of any one of claims 13-15, wherein step (i) comprises contacting the population of cells (e.g., T cells) with IL-2, IL-15, and IL-21, and wherein the percentage of PD-1+CD8+ T cells among CD8+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

17. The method of any one of claims 13-16, wherein step (i) comprises contacting the population of cells (e.g., T cells) with IL-2, IL-15, and IL-21, and wherein the percentage of LAG3+CD8+ T cells among CD8+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

18. The method of any one of claims 13-17, wherein step (i) comprises contacting the population of cells (e.g., T cells) with IL-2, IL-15, and IL-21, and wherein the percentage of PD-1+LAG3+CD8+ T cells among CD8+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

19. The method of any one of claims 13-18, wherein step (i) comprises contacting the population of cells (e.g., T cells) with IL-2, IL-15, and IL-21, and wherein the percentage of PD-1+CD4+ T cells among CD4+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

20. The method of any one of claims 13-19, wherein step (i) comprises contacting the population of cells (e.g., T cells) with IL-2, IL-15, and IL-21, and wherein the percentage of LAG3+CD4+ T cells among CD4+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

21. The method of any one of claims 13-20, wherein step (i) comprises contacting the population of cells (e.g., T cells) with IL-2, IL-15, and IL-21, and wherein the percentage of PD-1+LAG3+CD4+ T cells among CD4+ T cells in the population of expanded cells at the end of step (iv) is lower (e.g., at least 30, 35, 40, 45, 50, 55, 60, 65, 70, 75, 80, 85, 90 or 95% lower) than the corresponding value in a population of cells made by an otherwise similar method in which the population of cells is not contacted with IL-2, IL-15, or IL-21, or is contacted with IL-2 but not IL-15 or IL-21, e.g., as assessed using methods described in Example 1 with respect to FIGS. 8-9.

22. The method of any one of claims 13-21, wherein step (iii) comprises contacting the population of cells (e.g., T cells) with T Cell TransAct™, and wherein the expansion of the population of expanded cells at the end of step (iv) relative to the population of cells at the beginning of step (i) is greater (e.g., at least 20, 30, 40, 50, 60, 70, 80, 90, 100, 200, 300, 400, or 500% greater) than the expansion of a population of cells made by an otherwise similar method in which the population of cells is contacted with an agent that stimulates a CD3/TCR complex and comprises a bead, and/or an agent that stimulates a costimulatory molecule and comprises a bead, e.g., e.g., as assessed using methods described in Example 1 with respect to FIGS. 4-6, and 10.

23. The method of any one of claims 13-22, wherein step (iii) comprises contacting the population of cells (e.g., T cells) with T Cell TransAct™, and wherein the percentage of CD28+ T cells among T cells (e.g., the percentage of CD28+CD8+ T cells among CD8+ T cells, or the percentage of CD28+CD4+ T cells among CD4+ T cells) in the population of expanded cells at the end of step (iv) is higher (e.g., at least 30, 35, 40, 50, 60, 70, 80, 90, 100, 200, 300, 400, or 500% higher) than the corresponding value in a population of cells made by an otherwise similar method in which the population of cells is contacted with an agent that stimulates a CD3/TCR complex and comprises a bead, and/or an agent that stimulates a costimulatory molecule and comprises a bead, e.g., as assessed using methods described in Example 1 with respect to FIG. 7.

24. The method of any one of claims 13-23, wherein step (iii) comprises contacting the population of cells (e.g., T cells) with T Cell TransAct™, and wherein the percentage of exhausted T cells among T cells (e.g., the percentage of PD-1+CD8+ T cells among CD8+ T cells, the percentage of LAG3+CD8+ T cells among CD8+ T cells, the percentage of PD-1+LAG3+CD8+ T cells among CD8+ T cells, the percentage of PD-1+CD4+ T cells among CD4+ T cells, the percentage of LAG3+CD4+ T cells among CD4+ T cells, or the percentage of PD-1+LAG3+CD4+ T cells among CD4+ T cells) in the population of expanded cells at the end of step (iv) is lower (e.g., at least 30, 35, 40, 45, 50, 55, 60, 65, 70, 75, 80, 85, 90, or 95% lower) than the corresponding value in a population of cells made by an otherwise similar method in which the population of cells is contacted with an agent that stimulates a CD3/TCR complex and comprises a bead, and/or an agent that stimulates a costimulatory molecule and comprises a bead, e.g., as assessed using methods described in Example 1 with respect to FIGS. 8-9.

25. The method of claim 10, wherein step (i) comprises contacting the population of cells (e.g., T cells) with IL-2, IL-15, and IL-21, and step (iii) comprises contacting (e.g., binding) the population of cells (e.g., T cells) with T Cell TransAct™, and wherein the expansion of the population of expanded cells at the end of step (iv) relative to the population of cells at the beginning of step (i) is similar to or differs by no more than 5, 10, or 15% from the expansion of a reference population of cells (e.g., a population of cells from a healthy donor) made by the same method, e.g., as assessed using methods described in Example 1 with respect to FIG. 11.

26. The method of any one of claims 1-25, further comprising step (v):
contacting the population of cells (e.g., T cells) with IL-2 and one or both of: IL-15 and IL-21, e.g., contacting the population of cells (e.g., T cells) with IL-2, IL-15, and IL-21, at least 3 days after the beginning of step (i), e.g., at least 3, 4, 5, 6, 7, 8, 9, or 10 days after the beginning of step (i).

27. A method of making a population of cells (e.g., T cells) that express a chimeric antigen receptor (CAR), the method comprising:
(a) providing apheresis, e.g., leukapheresis, material, optionally wherein the apheresis, e.g., leukapheresis, material is cryopreserved after being isolated from a subject, and thawed prior to step (b),
(b) isolating a population of cells (e.g., T cells) from the apheresis, e.g., leukapheresis, material using negative selection, e.g., by reducing monocytes (e.g., CD14+ cells), B cells (e.g., CD19+ cells), and/or NK cells (e.g., CD56+ cells), from the apheresis, e.g., leukapheresis, material, e.g., using CliniMACS,
(c) contacting the population of cells (e.g., T cells) with IL-2 and one or both of: IL-15 and IL-21, e.g., contacting the population of cells (e.g., T cells) with IL-2, IL-15, and IL-21,
(d) contacting (e.g., binding) the population of cells (e.g., T cells) with an agent that stimulates a CD3/TCR complex and/or an agent that stimulates a costimulatory molecule, wherein the agent that stimulates a CD3/TCR complex or the agent that stimulates a costimulatory molecule does not comprise a bead, e.g., contacting (e.g., binding) the population of cells (e.g., T cells) with T Cell TransAct™,
(e) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(f) expanding the population of cells comprising the nucleic acid molecule for at least 3, 4, 5, 6, 7, 8, 9, or 10 days in vitro, e.g., expanding the population of cells comprising the nucleic acid molecule for about 4-10 days, to produce a population of expanded cells, optionally wherein:
the population of cells at the beginning of step (i) has one or more (e.g., 2, 3, 4, 5, 6, 7, 8, 9, 10, 11, 12, 13, 14, or all) of the following properties:
(1) the population of cells at the beginning of step (i) does not expand or expands for no more than 5, 6, 7, 8, or 9-fold over 8-11 days using the Bead CART cell manufacturing process described in Example 1,
(2) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than 5, 6, 7, 8, 9, 10, 15, 20, 25, 30, 40, 50, or 60%, e.g., lower than 10%,
(3) the percentage of naïve T cells and/or Tscm among T cells, the percentage of CD4+ naïve T cells and/or Tscm cells among CD4+ T cells, or the percentage of CD8+ naïve T cells and/or Tscm cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, or 90% lower than the corresponding value in a reference population of cells,
(4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than 20, 25, 30, 40, 50, 60, 70, 80, 90, or 95%, e.g., higher than 50%,
(5) the percentage of Teff cells and/or Tem cells among T cells, the percentage of CD4+ Teff cells and/or Tem cells among CD4+ T cells, or the percentage of CD8+ Teff cells and/or Tem cells among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 600, 700, 800 or 900% higher than the corresponding value in a reference population of cells,
(6) the percentage of CD28+CD4+ T cells among CD4+ T cells in the population of cells at the beginning of step (i) is no more than 40, 45, 50, 55, 60, 65, 70, 75, 80, 85, 90, or 95%, e.g., no more than 50%,
(7) the percentage of CD28+CD8+ T cells among CD8+ T cells in the population of cells at the beginning of step (i) is no more than 30, 35, 40, 45, 50, 55, 60, 65, or 70%, e.g., no more than 50%,
(8) the percentage of CD28+ T cells among T cells, the percentage of CD28+CD4+ T cells among CD4+ T cells, or the percentage of CD28+CD8+ T cells among CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CART cell manufacturing process described in Example 1), e.g., at least 20, 30, 40, 50, 60, 70, 80, or 90% lower than the corresponding value in a reference population of cells,
(9) the ratio of CD4+ T cells to CD8+ T cells in the population of cells at the beginning of step (i) is lower than 0.5, 0.8, 1, 1.2, or 1.5,
(10) the ratio of CD4+ T cells to CD8+ T cells in the population of cells at the beginning of step (i) is lower than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, or 99% lower than the corresponding value in a reference population of cells,
(11) the percentage of CD4+ senescent cells (e.g., CD28−CD27−CD57+CD4+ senescent cells) among CD4+ T cells in the population of cells at the beginning of step (i) is more than 2, 5, 10, 15, 20, 30, 40, 50, 60, 70, 80, 90 or 95%,
(12) the percentage of CD8+ senescent cells (e.g., CD28−CD27−CD57+CD8+ senescent cells) among CD8+ T cells in the population of cells at the beginning of step (i) is more than 20, 25, 30, 35, 40, 50, 60, 70, 80, 90 or 95%,
(13) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 60, 70, 80, 90 or 95% higher than the corresponding value in a reference population of cells,
(14) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than 10, 20, 30, 40, 50, 60, 70, 80 or 90%, and
(15) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells in the population of cells at the beginning of step (i) is higher than the corresponding value in a reference population of cells (e.g., a population of cells from a healthy donor, or a population of cells that expands more than 10, 15, or 20-fold over 8-11 days using the Bead CAR T cell manufacturing process described in Example 1), e.g., at least 20, 30, 40, 50, 60, 70, 80, 90, 100, 200, 300, 400, 500, 1000, 2000, 5000 or 9000% higher than the corresponding value in a reference population of cells.

28. A method of evaluating or predicting suitability of a population of cells (e.g., T cells) for chimeric antigen receptor (CAR) manufacturing, the method comprising:
acquiring a value for one or more (e.g., 2, 3, 4, 5, or all) of the following from the population of cells (e.g., T cells):
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells,
(2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells,
(3) the percentage of CD28+ T cells among T cells, the percentage of CD28+CD4+ T cells among CD4+ T cells, or the percentage of CD28+CD8+ T cells among CD8+ T cells,
(4) the ratio of CD4+ T cells to CD8+ T cells,
(5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and
(6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, wherein:
(a) a decrease in the value of one, two, or all of (1), (3), and (4) as compared to a reference value, e.g., a healthy donor reference value, is indicative or predicative of decreased suitability of the population of cells (e.g., T cells) for CAR manufacturing,
(b) an increase in the value of one, two, or all of (1), (3), and (4) as compared to a reference value, e.g., a healthy donor reference value, is indicative or predicative of increased suitability of the population of cells (e.g., T cells) for CAR manufacturing,
(c) an increase in the value of one, two, or all of (2), (5), and (6) as compared to a reference value, e.g., a healthy donor reference value, is indicative or predicative of decreased suitability of the population of cells (e.g., T cells) for CAR manufacturing, or
(d) a decrease in the value of one, two, or all of (2), (5), and (6) as compared to a reference value, e.g., a healthy donor reference value, is indicative or predicative of increased suitability of the population of cells (e.g., T cells) for CAR manufacturing,
thereby evaluating or predicting suitability of the population of cells (e.g., T cells) for CAR manufacturing.

29. The method of claim 28, comprising acquiring the value of (1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells, wherein the value being lower than 8, 9, 10, 11, 12, 15, or 20%, e.g., 10%, indicates or predicts:
decreased suitability of the population of cells (e.g., T cells) for CAR manufacturing, and/or
the population of cells (e.g., T cells) shows more expansion using the method of any one of claims 1-27, e.g., the bead-free stimulation and cytokine (BFSC) process described in Example 1, compared with the Bead CAR T cell manufacturing process described in Example 1.

30. The method of claim 28 or 29, comprising acquiring the value of (2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, wherein the value being higher than 40, 45, 50, 55, or 60%, e.g. 50%, indicates or predicts:
decreased suitability of the population of cells (e.g., T cells) for CAR manufacturing, and/or
the population of cells (e.g., T cells) shows more expansion using the method of any one of claims 1-27, e.g., the bead-free stimulation and cytokine (BFSC) process described in Example 1, compared with the Bead CAR T cell manufacturing process described in Example 1.

31. The method of any one of claims 28-30, comprising acquiring the value of (3) the percentage of CD28+ T cells among T cells, the percentage of CD28+CD4+ T cells among CD4+ T cells, or the percentage of CD28+CD8+ T cells among CD8+ T cells, wherein the value being lower than 40, 45, 50, 55, or 60%, e.g. 50%, indicates or predicts:
decreased suitability of the population of cells (e.g., T cells) for CAR manufacturing, and/or
the population of cells (e.g., T cells) shows more expansion using the method of any one of claims 1-27, e.g., the bead-free stimulation and cytokine (BFSC) process described in Example 1, compared with the Bead CAR T cell manufacturing process described in Example 1.

32. The method of any one of claims 28-31, comprising acquiring the value of (4) the ratio of CD4+ T cells to CD8+ T cells, wherein the value being lower than 0.5, 0.8, 1, 1.2, or 1.5 indicates or predicts decreased suitability of the population of cells (e.g., T cells) for CAR manufacturing.

33. The method of any one of claims 28-32, comprising acquiring the value of (5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, wherein the value being higher than 20, 25, 30, 35, 40, 50, 60, 70, 80, 90 or 95% indicates or predicts decreased suitability of the population of cells (e.g., T cells) for CAR manufacturing.

34. The method of any one of claims 28-33, comprising acquiring the value of (6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, wherein the value being higher than 10, 20, 30, 40, 50, 60, 70, 80 or 90% indicates or predicts decreased suitability of the population of cells (e.g., T cells) for CAR manufacturing.

35. A method of making a population of cells (e.g., T cells) that express a chimeric antigen receptor (CAR), the method comprising:
responsive to a decreased value for one, two, or all of the following in the population of cells (e.g., T cells):
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells,
(2) the percentage of CD28+ T cells among T cells, the percentage of CD28+CD4+ T cells among CD4+ T cells, or the percentage of CD28+CD8+ T cells among CD8+ T cells, and
(3) the ratio of CD4+ T cells to CD8+ T cells,
and/or responsive to an increased value of one, two, or all of the following in the population of cells (e.g., T cells):
(4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells,
(5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and
(6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells,
as compared to a reference value, e.g., a healthy donor reference value, performing:
(a) contacting the population of cells (e.g., T cells) with IL-2 and one or both of: IL-15 and IL-21, e.g., contacting the population of cells (e.g., T cells) with IL-2, IL-15, and IL-21,
(b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™,
(c) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells, optionally wherein:
performing the bead-free stimulation and cytokine (BFSC) process described in Example 1.

36. A method of making a population of cells (e.g., T cells) that express a chimeric antigen receptor (CAR), the method comprising:
responsive to:
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells (e.g., T cells) being lower than 8, 9, 10, 11, 12, 15, or 20%, e.g., 10%,
(2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells (e.g., T cells) being higher than 40, 45, 50, 55, or 60%, e.g. 50%, and/or
(3) the percentage of CD28+ T cells among T cells, the percentage of CD28+CD4+ T cells among CD4+ T cells, or the percentage of CD28+CD8+ T cells among CD8+ T cells in the population of cells (e.g., T cells) being lower than 40, 45, 50, 55, or 60%, e.g. 50%, performing:
(a) contacting the population of cells (e.g., T cells) with IL-2 and one or both of: IL-15 and IL-21, e.g., contacting the population of cells (e.g., T cells) with IL-2, IL-15, and IL-21,
(b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™,
(c) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells, optionally wherein:
performing the bead-free stimulation and cytokine (BFSC) process described in Example 1.

37. A method of making a population of cells (e.g., T cells) that express a chimeric antigen receptor (CAR), the method comprising:
responsive to:
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells in the population of cells (e.g., T cells) being higher than 8, 9, 10, 11, 12, 15, or 20%, e.g., 10%,
(2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells in the population of cells (e.g., T cells) being lower than 40, 45, 50, 55, or 60%, e.g. 50%, and/or
(3) the percentage of CD28+ T cells among T cells, the percentage of CD28+CD4+ T cells among CD4+ T cells, or the percentage of CD28+CD8+ T cells among CD8+ T cells in the population of cells (e.g., T cells) being higher than 40, 45, 50, 55, or 60%, e.g. 50%, performing:
(A) (a) contacting the population of cells (e.g., T cells) with IL-2 and one or both of: IL-15 and IL-21, e.g., contacting the population of cells (e.g., T cells) with IL-2, IL-15, and IL-21,
(b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™,
(c) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells, optionally wherein:
performing the bead-free stimulation and cytokine (BFSC) process described in Example 1, or
(B) the Bead CAR T cell manufacturing process described in Example 1.

38. A method of making a population of cells (e.g., T cells) that express a chimeric antigen receptor (CAR), the method comprising:
acquiring a value for one or more (e.g., 2, 3, 4, 5, or all) of the following from the population of cells (e.g., T cells):
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells,
(2) the percentage of CD28+ T cells among T cells, the percentage of CD28+CD4+ T cells among CD4+ T cells, or the percentage of CD28+CD8+ T cells among CD8+ T cells,
(3) the ratio of CD4+ T cells to CD8+ T cells,
(4) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells,
(5) the percentage of senescent T cells (e.g., CD28−CD27−CD57+ senescent T cells) among T cells, the percentage of CD4+ senescent cells (e.g., CD28−CD27−CD57+CD4+ senescent cells) among CD4+ T cells, or the percentage of CD8+ senescent cells (e.g., CD28−CD27−CD57+CD8+ senescent cells) among CD8+ T cells, and
(6) the percentage of exhausted T cells (e.g., PD-1+, LAG3+, or PD-1+LAG3+ T cells) among T cells, the percentage of exhausted CD4+ T cells (e.g., PD-1+CD4+, LAG3+CD4+, or PD-1+LAG3+CD4+ T cells) among CD4+ T cells, or the percentage of exhausted CD8+ T cells (e.g., PD-1+CD8+, LAG3+CD8+, or PD-1+LAG3+CD8+ T cells) among CD8+ T cells, wherein:
responsive to a decrease in the value of one, two, or all of (1)-(3), or responsive to an increase in the value of one, two, or all of (4)-(6), as compared to a reference value, e.g., a healthy donor reference value, performing:
(a) contacting the population of cells (e.g., T cells) with IL-2 and one or both of: IL-15 and IL-21, e.g., contacting the population of cells (e.g., T cells) with IL-2, IL-15, and IL-21,
(b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™,
(c) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells, optionally wherein:
performing the bead-free stimulation and cytokine (BFSC) process described in Example 1.

39. A method of making a population of cells (e.g., T cells) that express a chimeric antigen receptor (CAR), the method comprising:
acquiring a value for one or more (e.g., 2 or all) of the following from the population of cells (e.g., T cells):
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells,
(2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, and
(3) the percentage of CD28+ T cells among T cells, the percentage of CD28+CD4+ T cells among CD4+ T cells, or the percentage of CD28+CD8+ T cells among CD8+ T cells, wherein:
responsive to value (1) being lower than 8, 9, 10, 11, 12, 15, or 20%, e.g., 10%; value (2) being higher than 40, 45, 50, 55, or 60%, e.g. 50%; and/or value (3) being lower than 40, 45, 50, 55, or 60%, e.g. 50%, performing:
(a) contacting the population of cells (e.g., T cells) with IL-2 and one or both of: IL-15 and IL-21, e.g., contacting the population of cells (e.g., T cells) with IL-2, IL-15, and IL-21,
(b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™,
(c) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells, optionally wherein:
performing the bead-free stimulation and cytokine (BFSC) process described in Example 1.

40. A method of making a population of cells (e.g., T cells) that express a chimeric antigen receptor (CAR), the method comprising:
acquiring a value for one or more (e.g., 2 or all) of the following from the population of cells (e.g., T cells):
(1) the percentage of naïve T cells and/or stem cell-like memory T cells (Tscm) among T cells, the percentage of CD4+ naïve T cells and/or Tscm cells among CD4+ T cells, or the percentage of CD8+ naïve T cells and/or Tscm cells among CD8+ T cells,
(2) the percentage of effector T cells (Teff) and/or effector memory T cells (Tem) among T cells, the percentage of CD4+ Teff cells and/or Tem cells among CD4+ T cells, or the percentage of CD8+ Teff cells and/or Tem cells among CD8+ T cells, and
(3) the percentage of CD28+ T cells among T cells, the percentage of CD28+CD4+ T cells among CD4+ T cells, or the percentage of CD28+CD8+ T cells among CD8+ T cells, wherein:
responsive to value (1) being higher than 8, 9, 10, 11, 12, 15, or 20%, e.g., 10%; value (2) being lower than 40, 45, 50, 55, or 60%, e.g. 50%; and/or value (3) being higher than 40, 45, 50, 55, or 60%, e.g. 50%, performing:
(A) (a) contacting the population of cells (e.g., T cells) with IL-2 and one or both of: IL-15 and IL-21, e.g., contacting the population of cells (e.g., T cells) with IL-2, IL-15, and IL-21,
(b) optionally, contacting (e.g., binding) the population of cells (e.g., T cells) with an agent that stimulates a CD3/TCR complex and/or an agent that stimulates a costimulatory molecule, optionally wherein the agent that stimulates a CD3/TCR complex or the agent that stimulates a costimulatory molecule does not comprise a bead, optionally wherein the agent that stimulates a CD3/TCR complex comprises an anti-CD3 antibody covalently attached to a colloidal polymeric nanomatrix, and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™,
(c) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(d) optionally, expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells, optionally wherein:
performing the bead-free stimulation and cytokine (BFSC) process described in Example 1, or
(B) the Bead CAR T cell manufacturing process described in Example 1.

41. The method of any one of claim 1-27, 29-34, 37, or 40, wherein the Bead CAR T cell manufacturing process described in Example 1 comprises:
(a) providing an apheresis, e.g., leukapheresis, product,
(b) isolating a population of cells (e.g., T cells) from the apheresis product and contacting the population of cells (e.g., T cells) using anti-CD3 and anti-CD28 antibodies coupled to Dynabeads,
(c) contacting the population of cells (e.g., T cells) with IL-2,
(d) contacting the population of cells (e.g., T cells) with a nucleic acid molecule (e.g., a DNA or RNA molecule) encoding the CAR, thereby providing a population of cells (e.g., T cells) comprising the nucleic acid molecule, and
(e) expanding the population of cells comprising the nucleic acid molecule for at least 3, 4, 5, 6, 7, 8, 9, or 10 days in vitro, e.g., expanding the population of cells comprising the nucleic acid molecule for about 4-10 days in vitro, to produce a population of expanded cells.

42. The method of any one of claims 1-41, wherein the CAR comprises an antigen binding domain, a transmembrane domain, and an intracellular signaling domain.

43. The method of claim 42, wherein the antigen binding domain binds to an antigen chosen from: CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

44. The method of claim 42 or 43, wherein the antigen binding domain comprises a CDR, VH, VL, scFv or a CAR sequence disclosed herein.

45. The method of any one of claims 42-44, wherein the antigen binding domain comprises a VH and a VL, wherein the VH and VL are connected by a linker, optionally wherein the linker comprises the amino acid sequence of SEQ ID NO: 63 or 104.

46. The method of any one of claims 42-45, wherein:
(a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

47. The method of any one of claims 42-46, wherein the antigen binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

48. The method of any one of claims 42-47, wherein the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

49. The method of any one of claims 42-48, wherein the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signalling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

50. The method of any one of claims 42-49, wherein the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

51. The method of any one of claims 42-50, wherein the CAR further comprises a leader sequence comprising the amino acid sequence of SEQ ID NO: 1.

52. The method of claim 10, wherein the cancer is chronic lymphoblastic leukemia (CLL) or diffuse large B-cell lymphoma (DLBCL).

53. A population of CAR-expressing cells (e.g., autologous or allogeneic CAR-expressing T cells or NK cells) made by the method of any one of claim 1-27 or 35-52.

54. A pharmaceutical composition comprising the population of CAR-expressing cells of claim 53 and a pharmaceutically acceptable carrier.

55. A method of increasing an immune response in a subject, comprising administering the population of CAR-expressing cells of claim 53 or the pharmaceutical composition of claim 54 to the subject, thereby increasing an immune response in the subject.

56. A method of treating a cancer in a subject, comprising administering the population of CAR-expressing cells of claim 53 or the pharmaceutical composition of claim 54 to the subject, thereby treating the cancer in the subject.

57. The method of claim 56, wherein the cancer is a solid cancer, e.g., chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

58. The method of claim 56, wherein the cancer is a liquid cancer, e.g., chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

59. The method of any one of claims 55-58, further comprising administering a second therapeutic agent to the subject.

60. The population of CAR-expressing cells of claim 53 or the pharmaceutical composition of claim 54 for use in a method of increasing an immune response in a subject, said method comprising administering to the subject an effective amount of the population of CAR-expressing cells or an effective amount of the pharmaceutical composition.

61. The population of CAR-expressing cells of claim 53 or the pharmaceutical composition of claim 54 for use in a method of treating a cancer in a subject, said method comprising administering to the subject an effective amount of the population of CAR-expressing cells or an effective amount of the pharmaceutical composition.
